SEQUENCE LISTING (16 sequences)

SEQ ID NO: 1 - DNA, 28 bp, Artificial Sequence
  forward primer for verifying homozygous deletion of RKIP
gctttccaggcctctcagtgttcatcag

SEQ ID NO: 2 - DNA, 21 bp, Artificial Sequence
  reverse primer for verifying homozygous deletion of RKIP
cccgcccatcctgcccatagg

SEQ ID NO: 3 - DNA, 20 bp, Artificial Sequence
  GAPDH forward primer
tggcaaagtggagattgttg

SEQ ID NO: 4 - DNA, 20 bp, Artificial Sequence
  GAPDH reverse primer
cattatcggccttgactgtg

SEQ ID NO: 5 - DNA, 21 bp, Artificial Sequence
  BNP forward primer
ggatcggatccgtcagtcgtt

SEQ ID NO: 6 - DNA, 21 bp, Artificial Sequence
  BNP reverse primer
agacccaggcagagtcagaaa

SEQ ID NO: 7 - DNA, 27 bp, Artificial Sequence
  ANF forward primer
acagatctgatggatttcaagaacctg

SEQ ID NO: 8 - DNA, 21 bp, Artificial Sequence
  ANF reverse primer
agtgcggcccctgcttcctca

SEQ ID NO: 9 - DNA, 21 bp, Artificial Sequence
  COL3alpha1 forward primer
aaacagcaaattcacttacac

SEQ ID NO: 10 - DNA, 17 bp, Artificial Sequence
  COL3alpha1 reverse primer
acccccaatgtcatagg

SEQ ID NO: 11 - protein, 187 aa, Homo sapiens
MPVDLSKWSGPLSLQEVDEQPQHPLHVTYAGAAVDELGKVLTPTQVKNRPTSISWDGLDSGKLYTLVLTDPDAPSRKDPKYREWHHFLVVNMKGNDISSGTVLSDYVGSGPPKGTGLHRYVWLVYEQDRPLKCDEPILSNRSGDHRGKFKVASFRKKYELRAPVAGTCYQAEWDDYVPKLYEQLSGK

SEQ ID NO: 12 - DNA, 564 bp, Homo sapiens
atgccggtggacctcagcaagtggtccgggcccttgagcctgcaagaagtggacgagcagccgcagcacccgctgcatgtcacctacgccggggcggcggtggacgagctgggcaaagtgctgacgcccacccaggttaagaatagacccaccagcatttcgtgggatggtcttgattcagggaagctctacaccttggtcctgacagacccggatgctcccagcaggaaggatcccaaatacagagaatggcatcatttcctggtggtcaacatgaagggcaatgacatcagcagtggcacagtcctctccgattatgtgggctcggggcctcccaagggcacaggcctccaccgctatgtctggctggtttacgagcaggacaggccgctaaagtgtgacgagcccatcctcagcaaccgatctggagaccaccgtggcaaattcaaggtggcgtccttccgtaaaaagtatgagctcagggccccggtggctggcacgtgttaccaggccgagtgggatgactatgtgcccaaactgtacgagcagctgtctgggaagtag

SEQ ID NO: 13 - protein, 187 aa, Rattus norvegicus
MAADISQWAGPLSLQEVDEPPQHALRVDYGGVTVDELGKVLTPTQVMNRPSSISWDGLDPGKLYTLVLTDPDAPSRKDPKFREWHHFLVVNMKGNDISSGTVLSEYVGSGPPKDTGLHRYVWLVYEQEQPLNCDEPILSNKSGDNRGKFKVESFRKKYHLGAPVAGTCFQAEWDDSVPKLHDQLAGK

SEQ ID NO: 14 - DNA, 564 bp, Rattus norvegicus
atggccgccgacatcagccagtgggccgggccgctgtcattacaggaggtggatgagccgccccagcacgccctgagggtcgactacggcggagtaacggtggacgagctgggcaaagtgctgacgcccacccaggtcatgaatagaccaagcagcatttcatgggatggccttgatcctgggaagctctacaccctggtcctcacagaccccgatgctcccagcaggaaggaccccaaattcagggagtggcaccacttcctggtggtcaacatgaagggcaacgacattagcagtggcactgtcctctccgaatacgtgggctccggacctcccaaagacacaggtctgcaccgctacgtctggctggtgtatgagcaggagcagcctctgaactgtgacgagcccatcctcagcaacaagtctggagacaaccgcggcaagttcaaggtggagtccttccgcaagaagtaccacctgggagccccggtggccggcacgtgcttccaggcagagtgggatgactctgtgcccaagctgcacgatcagctggctgggaagtag

SEQ ID NO: 15 - protein, 187 aa, Mus musculus
MAADISQWAGPLCLQEVDEPPQHALRVDYAGVTVDELGKVLTPTQVMNRPSSISWDGLDPGKLYTLVLTDPDAPSRKDPKFREWHHFLVVNMKGNDISSGTVLSDYVGSGPPSGTGLHRYVWLVYEQEQPLSCDEPILSNKSGDNRGKFKVETFRKKYNLGAPVAGTCYQAEWDDYVPKLYEQLSGK

SEQ ID NO: 16 - DNA, 564 bp, Mus musculus
atggccgccgacatcagccagtgggccgggcccttgtgcttgcaggaggtggacgagccgccccagcacgccctgcgggtcgactacgccggggtgacggtggacgagctgggcaaagtgctaacgcccacccaggttatgaacaggcccagcagcatttcatgggacggccttgatcctgggaaactctacaccctggtcctcacagaccccgatgctcccagcaggaaggatcccaaattcagggagtggcaccacttcctggtggtcaacatgaagggtaatgacattagcagtggcactgtcctctcagattatgtgggctccgggcctcccagtggcacaggtctccaccgctatgtctggctggtgtacgagcaggaacagccgctgagctgcgacgagcccattctcagcaacaagtctggagacaatcgcggcaagttcaaggtggagaccttccgcaagaagtataacctgggagccccggtggcgggcacgtgctaccaagccgagtgggatgactatgtgcccaagctgtacgagcagctgtcagggaagtag

(19) United States
(12) Patent Application Publication
(10) Pub. No.: US 2018/0179292 A1
(43) Pub. Date: Jun. 28, 2018

(54) RKIP AGONISM IN THE TREATMENT AND PREVENTION OF HEART FAILURE

(71) Applicant: Leibniz-Institut für analytische Wissenschaften- ISAS-e.V., Dortmund (DE)

(72) Inventor: Kristina LORENZ, Würzburg (DE)

(21) Appl. No.: 15/555,725

(22) PCT Filed: Mar. 7, 2016
(86) PCT No.: PCT/EP2016/054794

§ 371 Date: Sep. 5, 2017

(30) Foreign Application Priority Data
Mar. 6, 2015 (EP) 15158087.5

(51) Int. Cl. C07K 16/28; A61K 38/00; A61P 9/04; C07K 16/22

(52) U.S. Cl. CPC C07K 16/2887; C07K 16/2863; C07K 16/22; A61P 9/04; A61K 38/005

(57) ABSTRACT

The present invention relates to a pharmaceutical composition comprising Raf kinase inhibitor protein (RKIP), or a fragment or a variant thereof; or an RKIP agonist for use in the treatment or prophylaxis of a low transvalvular pressure gradient (Pₘₑₐₙ) in a patient. The present invention further relates to a method of treatment of a patient in need thereof, said method comprising administering to said patient a medically active amount of a compound selected from the group consisting of: a Raf kinase inhibitor protein (RKIP) or a functional fragment or a functional variant thereof; and a RKIP agonist, wherein said patient suffers from or is likely to suffer from a low transvalvular pressure gradient (Pₘₑₐₙ). The present invention further relates to A pharmaceutical composition comprising an antagonist of CD20 and/or an anti-CD20 molecule; a VEGF antagonist; or an inhibitor of VEGF related tyrosine kinases for use in the treatment or prophylaxis of a heart failure. The present invention further relates to a corresponding method of treatment.

The present invention relates to a pharmaceutical composition comprising Raf kinase inhibitor protein (RKIP), or a fragment or a variant thereof; or an RKIP agonist for use in the treatment or prophylaxis of a low transvalvular pressure gradient in a patient. The present invention further relates to a method of treatment of a patient in need thereof, said method comprising administering to said patient a medically active amount of a compound selected from the group consisting of: a Raf kinase inhibitor protein or a functional fragment or a functional variant thereof; and a RKIP agonist, wherein said patient suffers from or is likely to suffer from a low transvalvular pressure gradient The present invention further relates to A pharmaceutical composition comprising an antagonist of CD20 and/or an anti-CD20 molecule; a VEGF antagonist; or an inhibitor of VEGF related tyrosine kinases for use in the treatment or prophylaxis of a heart failure. The present invention further relates to a corresponding method of treatment.

Heart failure is a major cause of morbidity and mortality in Western countries and its prevalence is continuously increasing¹. In heart failure, activation of the sympathetic nervous system augments cardiac contractility via β₁- and to some extent also via β₂-adrenoceptors in an attempt to maintain cardiac output. Therapeutically, β-adrenergic agonists like dobutamine are commonly used to stabilize patients in acute heart failure^{1,2}. For a better outcome in chronic heart failure, there is a need of approaches to improve cardiac contractility, but thus far there is no long-term positive inotropic strategy available that does not exacerbate cardiac deterioration and mortality. In particular, chronic stimulation of β₁AR induces structural cardiac damage, involving cardiac hypertrophy, cardiomyocyte apoptosis and interstitial fibrosis¹⁻³. Several lines of evidence, notably transgenic mouse lines, suggest that increased β₂AR activity, in contrast to increased β₁AR activity, is chronically much better tolerated and may even be beneficial⁴⁻⁸.

In addition to the detrimental effects mentioned above, chronic βAR stimulation initiates desensitization of βAR via G protein-coupled receptor kinases (GRKs) and β-arrestins, thereby limiting the possibilities to directly restore β-adrenergic function. Interestingly, currently discussed positive inotropic strategies that might prove protective in chronic heart failure include increased β-adrenergic stimulation via inhibition of GRK2^{6,9-14}.

The mode of βAR activation (βAR activation by agonists/catecholamines vs. inhibition of βAR desensitization) is considered in the art as decisive for cardiac adverse effects of βAR signaling. However, conclusive data thereon are still missing and corresponding molecular mechanisms and underlying biology remains elusive.

One mode of promoting βAR activity might involve the Raf kinase inhibitor protein (RKIP) in part via its inhibition of GRK2¹⁵. In its basal state, RKIP inhibits the kinase Raf1¹⁶, but it binds to the N-terminus of GRK2 after phosphorylation on position 153 by PKC^{15,17} and thereby inhibits GRK2. Furthermore, the N-terminus of GRK2 is important for binding to its receptor substrates¹⁸. In the heart, RKIP exists mostly in the serine-153-phosphorylated state and is, thus, predominantly bound to GRK2¹⁷.

In in vivo models, Lorenz et al. had found that RKIP may trigger cardiac hypercontractiltiy without apparent effects on the aging or pressure overloaded heart; see Proceedings of the British Pharmacological Society 2012 Vol. 10. 1. Abstract 36P. It could also be shown that cardiac overexpression of RKIP prevented left ventricular dilatation and loss of contractile function in a mouse model of pressure overload-induces heart failure. It was, accordingly, speculated that RKIP may represent a potential therapeutic principle in heart failure.

However, the prior art did not provide for potential treatment options in specific heart failure patients. Accordingly, the technical problem underlying the present invention is the provision of patients or patient groups that would benefit from medical intervention with RKIP and/or RKIP agonists.

The solution to said technical problem is provided herein below and characterized in the appended claims.

Accordingly, the present invention relates to a pharmaceutical composition comprising Raf kinase inhibitor protein (RKIP), a fragment or a variant thereof, or an RKIP agonist for use in the treatment or prophylaxis of a low (aortic) transvalvular pressure gradient (Pₘₑₐₙ) in a patient; and/or for use in treating or protecting a patient suffering from or will likely suffer from a low (aortic) transvalvular pressure gradient.

In other words, both of the following in envisaged in the context of the invention:

First, the treatment or prophylaxis of a low aortic transvalvular pressure gradient (Pₘₑₐₙ) in a patient. In the context of this treatment, it is envisaged to increase the low aortic transvalvular pressure gradient, preferably to a non-pathological degree.
Second, the treatment or protection of a patient suffering or being expected to suffer from a low aortic transvalvular pressure gradient. In the context of this treatment, it is preferred to increase the low aortic transvalvular pressure gradient, more preferably to a non-pathological degree. However, in the context of this treatment, the low aortic transvalvular pressure gradient may not necesarily be increased.

In one aspect, the patient to be treated in accordance with the invention suffers from chemical cardiomyopathy, ischemia and/or, preferably, (severe) aortic valve stenosis (AS). In another aspect, the disease to be treated in the context of the invention is chemical cardiomyopathy, ischemia and/or, preferably, (severe) AS. In a more particular aspect, the (severe) AS is low gradient AS (LG/AS).

In light of this embodiment, the present invention also relates to a method of medical intervention/treatment of a human patient in need of such intervention/treatment, said method comprising the step of administering to said patient a medically active amount of a compound selected from the group consisting of:
a Raf kinase inhibitor protein (RKIP) or a functional fragment or a functional variant thereof (or an nucleic acid molecule coding for said RKIP or said functional fragment of functional derivative); and
a RKIP agonist;
   wherein said patient already suffers from a (pathologically) low aortic transvalvular pressure gradient (Pmean) or wherein said patient is prophylactically be treated since he/she is likely to suffer a (pathologically) low aortic transvalvular pressure gradient in a patient.

Accordingly, the present invention provides for means and methods of medical intervention in patients with low aortic transvalvular pressure gradient (Pmean). These patients are preferably human patients who suffers from (severe) aortic valve stenosis (AS), preferably low gradient AS (LG/AS), chemical cardiomyopathy and/or ischemia. A low (aortic) transvalvular pressure gradient (Pₘₑₐₙ) can be established with methods known in the art and as documented and illustrated herein. Patients who benefit from the herein proposed pharmaceutical compositions and method of treatments are individuals who have transvalvular pressure gradient of lower or equal to 60 mmHg, preferably lower or equal to 50 mmHg, preferably lower or equal to 40 mmHg (most preferred) or even lower or equal to 30 mmHg as, for example, measured by combined two-dimensional echocardiography and continuous wave Doppler recordings.

Corresponding relevant clinical data are also provided in the enclosed technical and experimental part of this invention.

In contrast to the initial observation that RKIP expression is up-regulated in heart failure in general, we now interestingly found that there is a specific entity of patients (e.g. low gradient aortic stenosis patients (and ischemic cardiomyopathy)) where RKIP expression and/or activation is not up-regulated (expression RKIP in low gradients) or even down-regulated (RKIP activation) and, thus, the counter-regulative, protective mechanism of RKIP up-regulation does not seem to be present or functioning. The extent of RKIP expression/activation is closely correlated to the cardiac function: for a fair comparison we compared patients with severe aortic stenosis with high and low expression/activation of RKIP (i.e. high and low gradient patients) and correlated RKIP expression/activation. Interestingly, patients with low transvalvular gradients have previously been shown to have high levels of interstitial fibrosis (Ruppert et al., 2013; Hemnann et al., 2011) and low gradients habe been identified as independent predictor of reduced cardiac event free survival and mortality (Lancelloti et al., 2012) and have low survival after aortic valve replacement (Hellmann et al., 2011). The treatment strategy “RKIP” seems thus of particular importance in low-gradient patients. In particular, it is envisaged with respect to this patients (i) to ameliorate cardiac function, (ii) increase β-adrenergic receptor signal integrity (iii) to improve interstitial fibrosis and/or (iv) provide an alternative strategy to treat this group of patients. Up to now, this group of patients has poor clinical outcome.

The present invention further relates to the following:

A pharmaceutical composition comprising RKIP or an RKIP agonist

for use in
(i) treating, protecting from or preventing heart failure;
(ii) long-term increase of cardiac contractile force;
(iii) maintaining cardiac function;
(iv) treating, protecting from or preventing pressure overload-induced cardiac failure;
(v) prophylaxis of heart failure;
(vi) induce (persistently) increased cardiac contractility;
(vii) induce persistent positive inotropy;
(viii) treating or preventing irregular heartbeat/arrhythmia (preferred)
(ix) reducing (effects of) pathological hypertrophy;
(x) inducing or increasing (effects of) physiological hypertrophy; and/or
(xi) converting (effects of) pathological hypertrophy into (effects of) physiological hypertrophy
   in a patient. In accordance with this pharmaceutical composition, said heart failure is and/or said patient may suffer from or may likely suffer from a (pathologically) low (aortic) transvalvular pressure gradient. The patient may also suffer from or may likely suffer from (severe) aortic valve stenosis (AS), chemical cardiomyopathy and/or ischemia and/or, preferably, low gradient AS (LG/AS).

In particular, the low (aortic) transvalvular pressure gradient (Pₘₑₐₙ) in accordance with the invention is envisaged to be pathologically low. Low, in particular pathologically low, means ≤ or <60 mmHg, preferably ≤ or <50 mmHg, preferably ≤ or <40 mmHg (most preferred) or even ≤ or <30 mmHg. This can, for example, be measured by combined two-dimensional echocardiography and continuous wave Doppler recordings as known in the art.

The aortic valve area in accordance with the invention may be small, in particular pathologically small, i.e. smaller as in a healthy patient. Small, in particular pathologically small, means ≤ or <1.4 cm², preferably ≤ or <1.4 cm², preferably ≤ or <1.0 cm² (most preferred) or even ≤ or <0.8 cm². The aortic valve area can, for example, be measured by relying on respective means and meathods known in the art and described herein.

In one aspect, the heart failure is and/or the patient suffers from or likely suffers from low-flow/low-gradient stenosis. For example, such a heart failure or patient has reduced ejection fraction (EF) (e.g. as compared to the EF of a healthy patient (about 60-75%)), a low (aortic) transvalvular pressure gradient (e.g. <40 mmHg), and/or a low aortic valve area (e.g. <1.0 cm²).

In accordance with the invention, the RKIP, a fragment or a variant thereof may be a polypeptide selected from the group consisting of
(a) a polypeptide which comprises or consists of the amino acid sequence as depicted in SEQ ID NO. 11, 13 or 15 or as available via the database entry NCBI Reference Sequence: NP_002558.1 (human); UniProt ID: P30086 (human); NCBI Reference Sequence: NP_058932.1 (rat) or UniProt ID: P31044-1 (rat);
(b) a polypeptide which comprises or consists of an amino acid sequence being at least 30%, preferably at least 40%, preferably at least 50%, preferably at least 60%, preferably at least 70%, preferably at least 80%, preferably at least 85%, preferably at least 90%, preferably at least 95%, preferably at least 97%, preferably at least 98%, preferably at least 99%, identical to the a polypeptide of (a). This polypeptide is preferably capable to increase (modest) (cardiac) expression of RKIP or of an RKIP agonist in accordance with the invention;
(c) a polypeptide which comprises or consists of an amino acid sequence encoded by a nucleic acid molecule hybridizing to the complementary strand of a nucleic acid molecule (e.g. as depicted in SEQ ID NO. 12, 14, 16) encoding the polypeptide of (a) or (b). This polypeptide is preferably capable to increase (modest) (cardiac) expression of RKIP or of an RKIP agonist in accordance with the invention;
(f) a polypeptide which comprises or consists of a fragment of the polypeptide of any one of (a) to (c). This polypeptide is preferably capable to increase (modest) (cardiac) expression of RKIP or of an RKIP agonist in accordance with the invention.

A fragment or variant of the RKIP or of an RKIP agonist in accordance with the invention is envisaged to be a biologically functional fragment or variant, e.g. a fragment of variant which is capable to increase (cardiac) expression, preferably modest (cardiac) expression, of RKIP or of an RKIP agonist in accordance with the invention. Such a fragment (or variant) may consist of at least 50, 150, 200, 250, 300, 350 or 400 amino acids. It is envisaged that the fragment or variant comprises the biologically functional part of RKIP. In particular, it is envisaged that the biologically functional part of RKIP is the part that interacts with GRK2. Without being bound by theory, for GRK2/RKIP interaction, RKIP has to be phosphorylated at a Ser residue which corresponds to Ser153 of the rat RKIP sequence. This leads to RKIP dimerization. Again without being bound by theory, this generates a new interaction interface consisting of RKIP surface or RKIP/RKIP surface. Hence, in one aspect, A fragment or variant of the RKIP or of an RKIP agonist in accordance with the invention comprises the mentioned Ser residue and/or the new interaction interface consisting of RKIP surface or RKIP/RKIP surface, or at least a part of this new interaction interface.

The RKIP agonist to be used in accordance with the invention may be selected from the group consisting of
(a) RKIP, the fragment or the variant thereof as defined herein elsewhere;
(b) a nucleic acid molecule encoding RKIP, the fragment or the variant thereof as defined herein elsewhere;
(c) a (n expression) vector comprising the nucleic acid molecule of (b);
(d) an antagonist of CD20 and/or an anti-CD20 molecule (e.g. Rituximab), an VEGF inhibitor/antagonist (e.g. Bevacizimab) or an inhibitor of VEGF related tyrosine kinases (e.g. Sorafenib (Nexavar®)); and
(e) an amino acid sequence that (agonistically) interacts with RKIP (e.g. a ligand or interacting antibody).

The RKIP and RKIP agonist and the fragment or variant thereof in accordance with the invention is envisaged to have one or more of the following functions:
(i) capability to long-term increase of cardiac contractile force;
(ii) capability to maintaining cardiac function;
(iii) capability to induce (persistently) increased cardiac contractility;
(iv) capability to induce persistent positive inotropy;
(v) capability to reduce (effects of) pathological hypertrophy;
(vi) capability to induce or increase (effects of) physiological hypertrophy;
(vii) capability to convert (effects of) pathological hypertrophy into (effects of) physiological hypertrophy;
(viii) capability to increase (cardiac) expression and/or activation, preferably modest (cardiac) expression and/or activation (e.g. 1, 2, 3, 4, 5, 6, 7, 8, 9 or 10 fold), of RKIP, for example of endogenous RKIP;
(ix) capability to increase or induce phosphorylation of RKIP, for example of endogenous RKIP. Such a phosphorylation results in an activation of RKIP, in particular, in an activation of RKIP as a GRK2-Inhibitor; and/or
(x) capability to activate β2AR, in particular β2AR in the Gi mode.

In a preferred aspect, it is envisaged in accordance with the invention to increase/induce the activated form of RKIP, i.e. the activation of RKIP, respectively. In this context, the RKIP may be the endogenous RKIP. Hence, preferably, the RKIP agonist and the fragment or variant thereof in accordance with the invention is envisaged to have the capability to increase/induce the activated form of (endogenous) RKIP, i.e. the activation of (endogenous) RKIP, respectively.

Activation of RKIP in accordance with the invention may be achieved by phosphorylation, in particular by phosphorylation at a Ser residue which corresponds to Sen 53 of the rat RKIP sequence (SEQ ID NO. 13). Without being bound by theory, this leads to RKIP dimerization and generates a new interaction interface consisting of RKIP surface or RKIP/RKIP surface. Again without being bound by theory, via this interaction phase, interaction with and, hence, inhibition of GRK2 takes place. The activation of RKIP is, for example, also described in Lorenz (Nature 426, 574-579, 2003) and Deiss (J Biol Chem 287, 23407-23417, 2012). The skilled person is readily able to test the (degree of) activation of RKIP by relying on respective means and methods well known in the art (see, for example, the above two papars of Lorenz and Deiss) an disclosed herein (see, for example, the appended examples).

In one aspect, the present invention relates to a pharmaceutical composition comprising Raf kinase inhibitor protein (RKIP), a fragment or a variant thereof, or an RKIP agonist for use in the prophylaxis or prevention of a low (aortic) transvalvular pressure gradient (Pₘₑₐₙ) in a patient; and/or

for use in treating or protecting a patient who will likely suffer from a low (aortic) transvalvular pressure gradient.

The skilled person is readily able to test whether a patient is in need of and/or can be treated by the prophylactive, preventive and/or protective medical intervention in accordance with the invention. Therby, the skilled person can rely on respective means and methods known in the art and disclosed herein. In this context, the skilled person can test one or more indications for a need of the prophylactive, preventive and/or protective medical intervention in accordance with the invention. Non-limiting examples of such indications are:

symptoms (e.g. Angina (breast pain), syncope, heart failure; small aortic valve area (e.g. <1.0 cm²); low transvalvular ressure gradient (e.g. <40 mmHg); and LV-hypertrophy. The latter three may be diagnosed by echocardiography for assessment of the degree of calcification, LV function and wall thickness and transvalvular gradient.

The pharmaceutical composition may be administered so that it is, or its active ingredient, is targeted to the heart/myocardium.

The pharmaceutical composition (the active ingredient) may be administered by gene therapy. This may be a lentivirus vector-mediated gene therapy (e.g. as review by Tilemann et al., 2012). More particular, this may be an AAV-mediated gene therapy.

The pharmaceutical composition (the active ingredient) may be administered by an adeno-associated virus vector.

The adeno-associated virus vector may be an adeno-associated virus serotype 9 (AAV9) vector.

The pharmaceutical composition may be administered chronically/in a long-term mode and/or at a low dose.

The administration in a chronic/long-term mode may be an administration every 1-4, preferably 2-3, weeks or even only every 2-3 months over a period of at least 1, 3 or 6 month, up to 1, 2, 3, 4, 5, 6, 7, 8, 9, 10 ore even more years.

The administration at a low dose may be a dose (of the pharmaceutical composition or the active ingredient) of about 1 to 50 mg/kg, 5 to 20 mg/kg, 8 to 17 mg/kg, 10 to 15 mg/kg bodyweight (bw) every 1-4, preferably 2-3, weeks or even only every 2-3 months.

Preferably, the pharmaceutical composition (the active ingredient) may be administered every 2 weeks at a dose of 10 mg/kg or every 3 weeks at a dose of 15 mg/kg.

For CD20 inhibitors/antagonists like rituximab, the pharmaceutical composition (the active ingredient) may be administered every 2-3 months at one of the above doses (e.g. 10 mg/kg).

The pharmaceutical composition (the active ingredient) may be administered so that an increase of (cardiac) RKIP activity/expression and/or an activation of (cardiac) RKIP is achieved.

The increase of (cardiac) RKIP activity/expression and/or an activation of (cardiac) RKIP may be 1-15 fold (cardiac) RKIP activity/expression in the patient.

A patient in accordance with the invention may be a mammal, preferably, a human.

In one particular aspect of this invention, the RKIP (e.g. cardiac RKIP) activity and/or expression and/or the activation of RKIP (e.g. cardiac RKIP) is reduced in the patient and/or in the context of the disease/failure to be treated in accordance with the invention. For example, the RKIP (e.g. cardiac RKIP) activity and/or expression and/or the activation of RKIP (e.g. cardiac RKIP) in the patient and/or in the context of the disease/failure to be treated in accordance with the invention is 1, 2, 3, 4, 5, 6, 7, 8, 9, 10, 11, 12, 13, 14 or 15 times less as compared to a healthy subject.

In a particularly preferred aspect of the invention, it is envisaged that antagonists of CD20 and/or anti-CD20 molecules are used in the medical intervention of heart failures and heart disorders as defined herein. These anti-CD20 molecules/CD20 antagonists may also comprise antibodies. Exemplified antibody in this context may be Rituximab, Ocrelizumab (see, inter alia, Kausar (2009), Expert Opinion Biol. Therapy 9: 889-95; Hutas (2008) Current Opinion Investigational Drugs 9: 1206-15), Ofatumumab (see, inter alia, Coiffier (2008) Blood 111 (3): 1094-100; Zhang (2009) MAbs 1: 326-31) or Obinutuzumab (see, inter alia, Robak (2009) Current Opinion Investigational Drugs 10 (6): 588-96). Other examples of anti-CD20 molecules/CD20 antagonists are Tositumomab (Bexxar), Ofatumumab (Arzerra) and veltuzumab (Immumunomedics). As is evident from the enclosed examples, it could surprisingly be shown that anti-CD20 molecules like Rituximab can up-regulate RKIP expression in vitro in cardiomyocytes. It is in particular documented that anti-CD20 (e.g. Rituximab) can significantly activate RKIP and increase so called “active” RKIP, respectively (“active” RKIP comprises a phosphorylation at a Ser residue which corresponds to Ser153 of the rat RKIP sequence (SEQ ID NO. 13)). What has been said with respect to RKIP activation herein elsewhere applies here, mutatis mutandis. Accordingly, it can surprisingly be shown that anti-CD20 can be plausibly used in the medical intervention of heart failures and heart disorders as described herein.

Similarly, it is also envisaged and documented herein that therapeutics that function as VEGF (Vascular Endothelial Growth Factor) antagonists can be employed in the medical uses and methods of treatment of this invention. In this context, also antibodies/antibody derivatives may be employed. One particular example of such an antibody is Bevacizumab (Avastin®, a recombinant humanized monoclonal antibody; see, inter alia, Los (2007) The Oncologist 12: 443-50). Bevacizumab is known to function as an angiogenesis inhibitor that specifically blocks the biological effects of vascular endothelial growth factor (VEGF). VEGF signals through VEGF Receptor 2 (VEGFR-2). VEGFR-2 is the major VEGF signalling receptor that mediates sprouting angiogenesis. Bevacizumab is a recombinant humanized monoclonal IgG1 antibody that directly binds to VEGF extracellularly to prevent interaction with VEGF receptors on the surface of endothelial cells. Thus, Bevacizumab in known in the art to inhibit the angiogenic activity of VEGF, a function that is medically used in the art in the treatment of cancers and certain eye-diseases, like age-related macular degeneration (AMD) or diabetic retinopathy. In the appended examples, it is documented that an anti-VEGF antibody/Bevacizumab does not only significantly activate RKIP and increase the level of “active” RKIP, respectively (“active” RKIP comprises a phosphorylation at a Ser residue which corresponds to Ser153 of the rat RKIP sequence (SEQ ID NO. 13)), but also the induction of RKIP in an experimental model of neonatal rat cardiomyocytes could be documented. What has been said with respect to RKIP activation herein elsewhere applies here, mutatis mutandis. In accordance with this invention, it is, therefore also proposed to employ anti-VEGF molecules, like anti-VEGF antibodies in the medical intervention/treatment of heart failure/heart disorders. Such anti-VEGF molecules may comprise antibodies, like the above described Bevacizumab, or antibody derivatives such as Ranibizumab (Lucentis 8).

Similarly, it is also envisaged and documented herein that therapeutics that function as inhibitors of VEGF related tyrosine kinases can be employed in the medical uses and methods of treatment of this invention. In this context, also small molecule tyrosin kinase inhibitors may be employed. Particular examples of the tyrosin kinase inhibitors in accordance with the invention are Sunitinib (Sutent®), Lapatinib (Tykerb®) or Sorafenib (Nexavar®) (Giovannetti, Curr Pharm Des 19(5), 2013, 927-39).

Since it is documented in the appended examples that anti-CD20 molecules (like Rituximab) or anti-VEGF molecules (like Bevacizumab) lead to an surprising increase of RKIP (and, accordingly, function as RKIP activators/agonists), it is made plausible in context of this invention that these compounds can successfully and surprisingly be employed in the medical intervention of heart failure/heart disease. In context of this invention and in a preferred embodiment of this invention, anti-CD20 molecules (like Rituximab) or anti-VEGF molecules (like Bevacizumab) are to be used in the treatment or prophylaxis of a (pathologically) low (aortic) transvalvular pressure gradient (Pmean); and/or in the medical intervention of a patient suffering from said low (aortic) transvalvular pressure gradient. As documented herein, these anti-CD20 molecules (like Rituximab) or anti-VEGF (like Bevacizumab) molecules can in particular be employed in the treatment of aortic valve stenosis (AS), in particular severe aortic valve stenosis, more preferably low gradient AS (LG/AS), chemical cardiomyopathy and/or ischemia. Preferably, the patients to be treated with RKIP agonist are those patients who suffer from or have a (pathologically) low (aortic) transvalvular pressure gradient and/or intrinsically low RKIP levels (RKIP expression and/or activity). As documented herein, CD20 antagonists (like Rituximab) as well as VEGF antagonist (like Bevacizumab) plausibly function as RKIP agonists and can therefore be surprisingly be used in the medical interventions as described herein. Accordingly, the present invention also relates to a method of treatment of patients suffering from or displaying a (pathologically) low (aortic) transvalvular pressure gradient, wherein said method comprises the step of administering to said patient in need of medical intervention a medically active amount of an anti-CD20 molecules or an anti-VEGF molecules. In one embodiment, said molecules are antibody molecules, like in case of anti-CD20 Rituximab of in case of anti-VEGF Bevacizumab. Yet, the present invention is not limited to these specific CD20 antagonists or VEGF antagonists. In a preferred embodiment of the CD20 antagonists or the VEGF antagonists to be employed in the medical interventions described herein, are molecules elicit RKIP activation (RKIP expression and/or activity).

In this specification, a number of documents including patent applications are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The invention will now be described by reference to the following figures and examples which are not to be construed as a limitation of the scope of the present invention.

The Figures and Tables show:

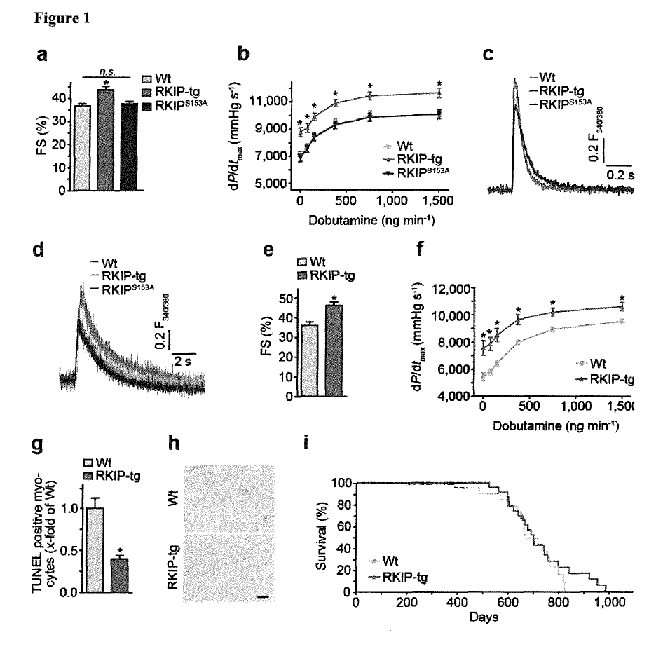

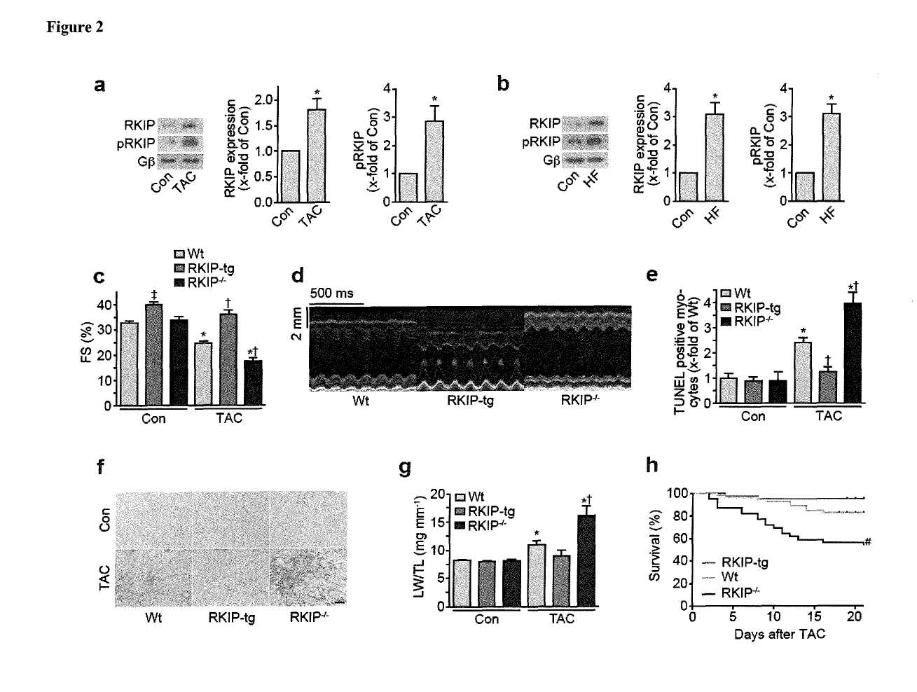

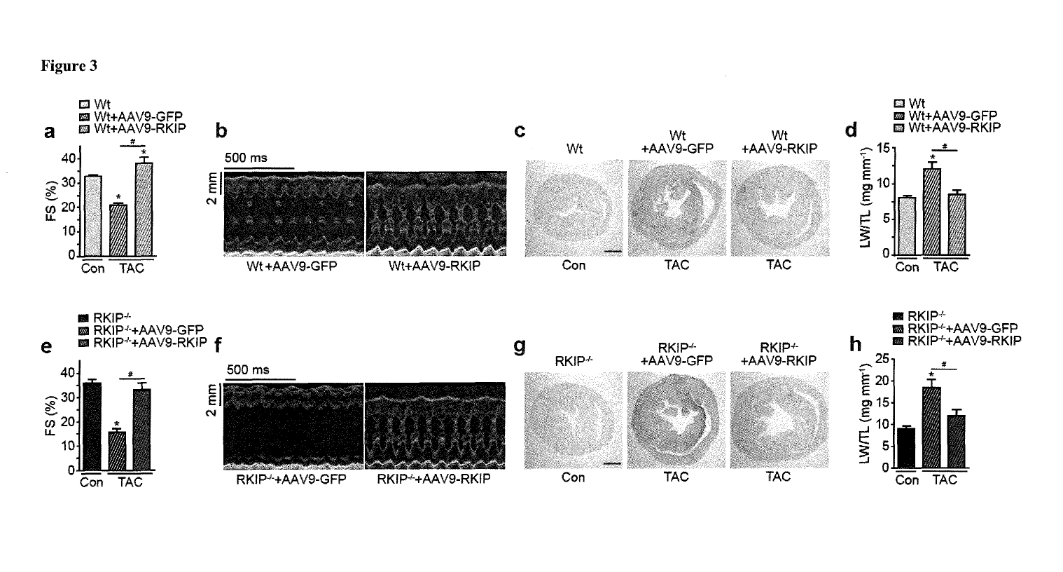

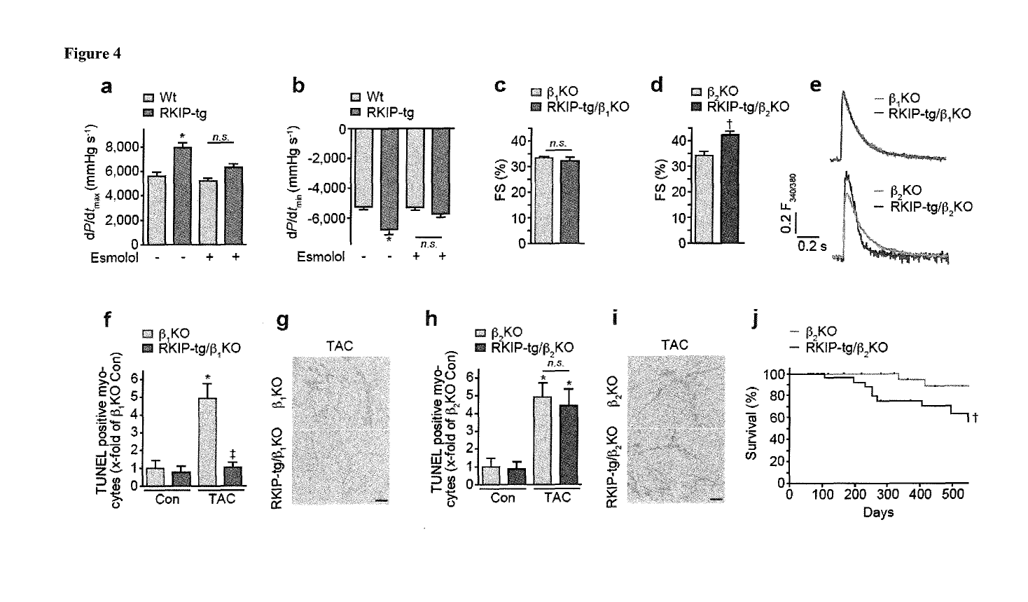

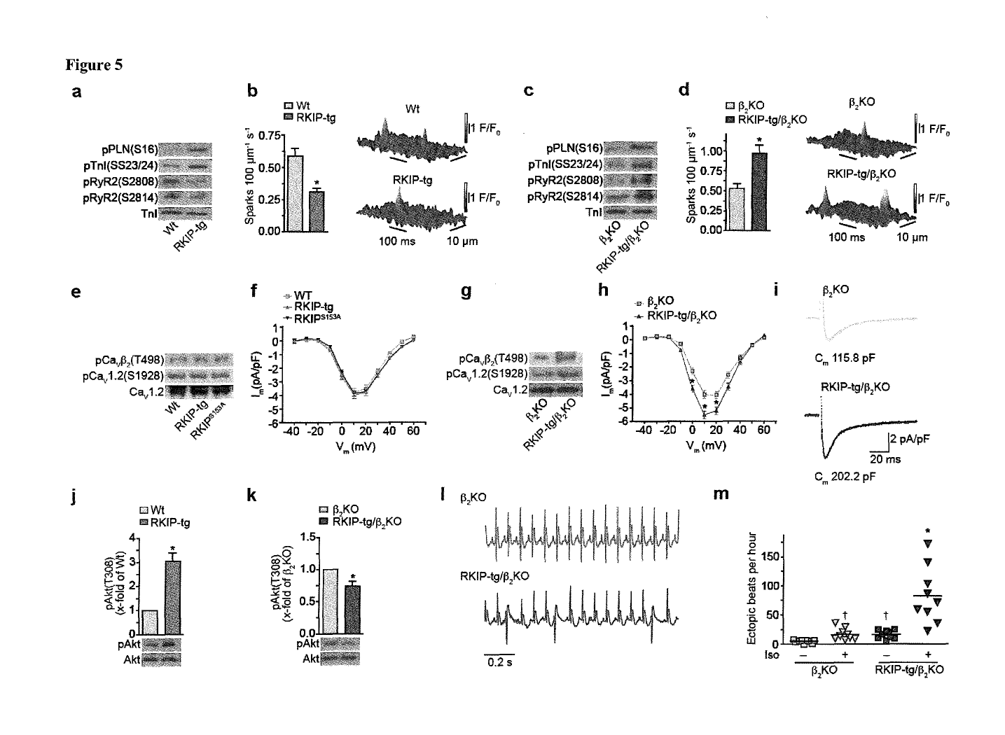

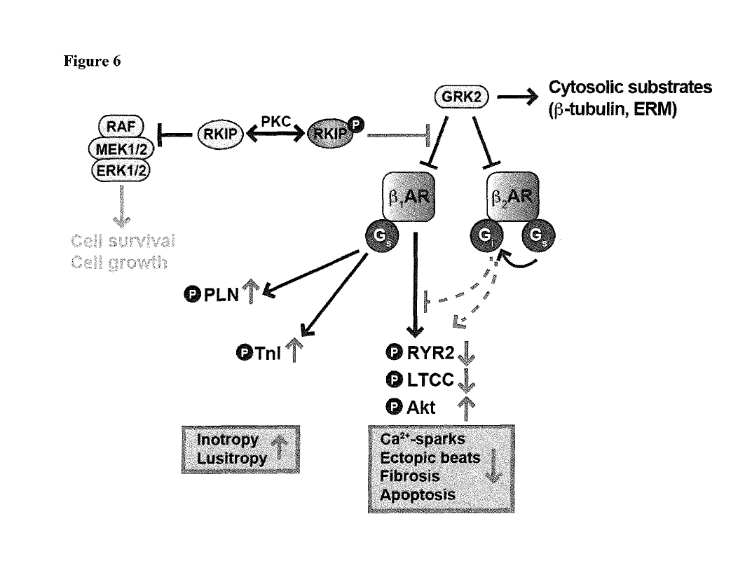

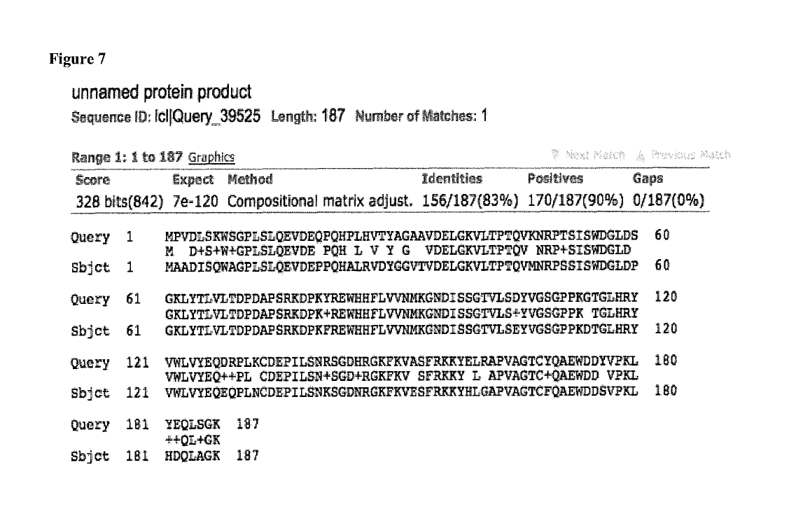

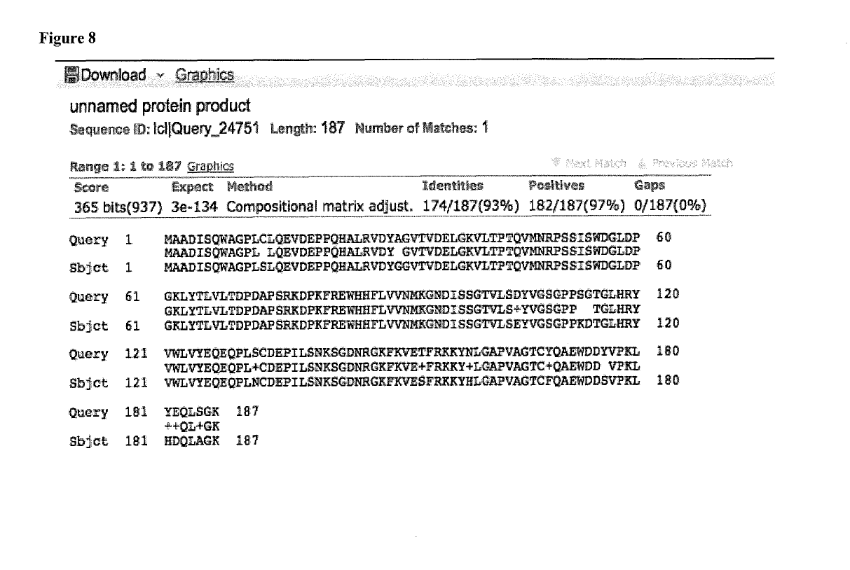

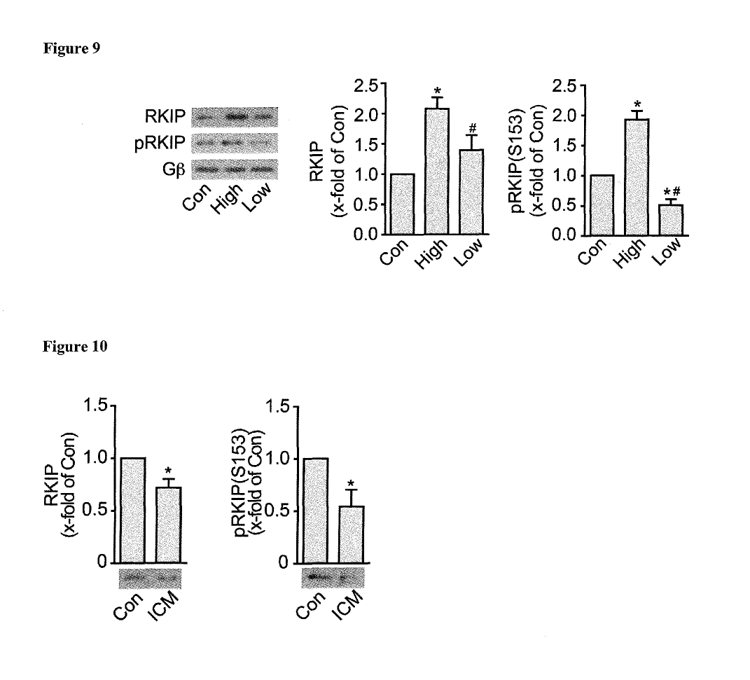

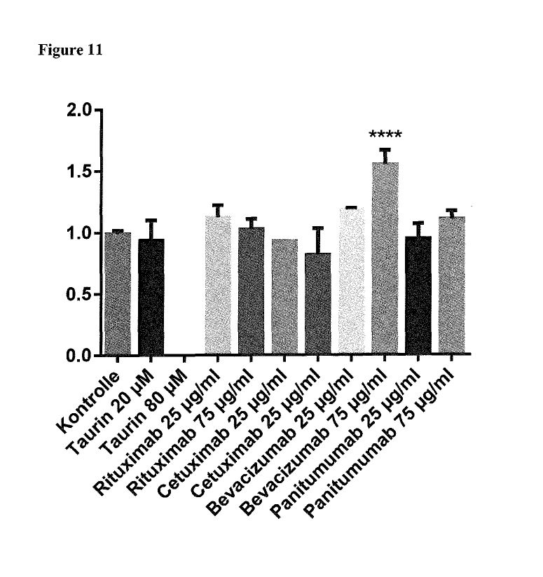

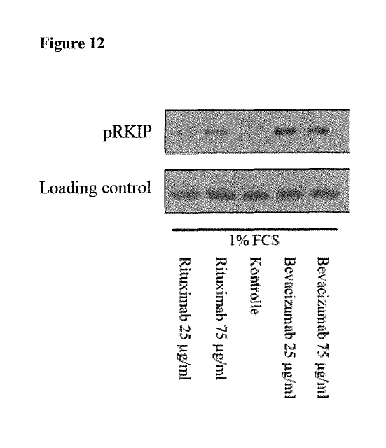

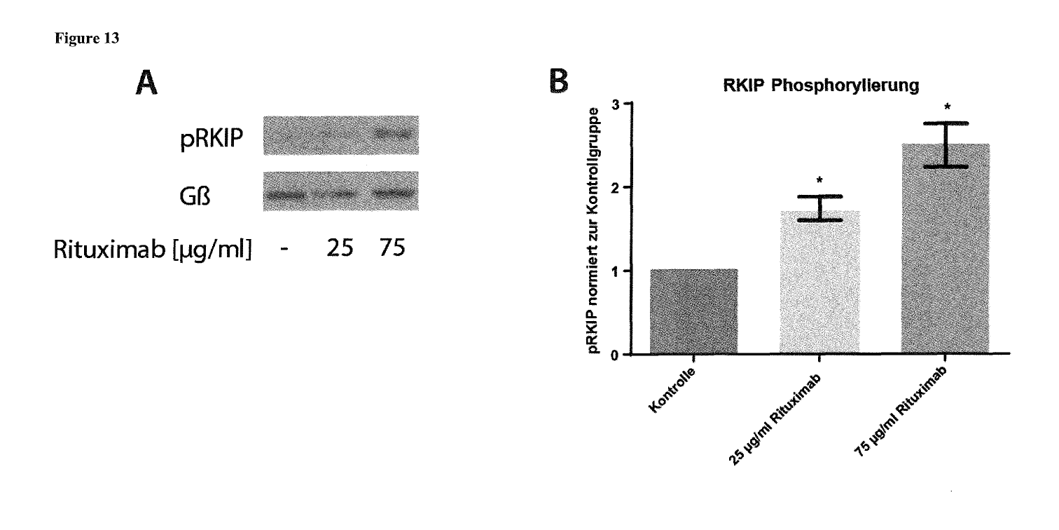

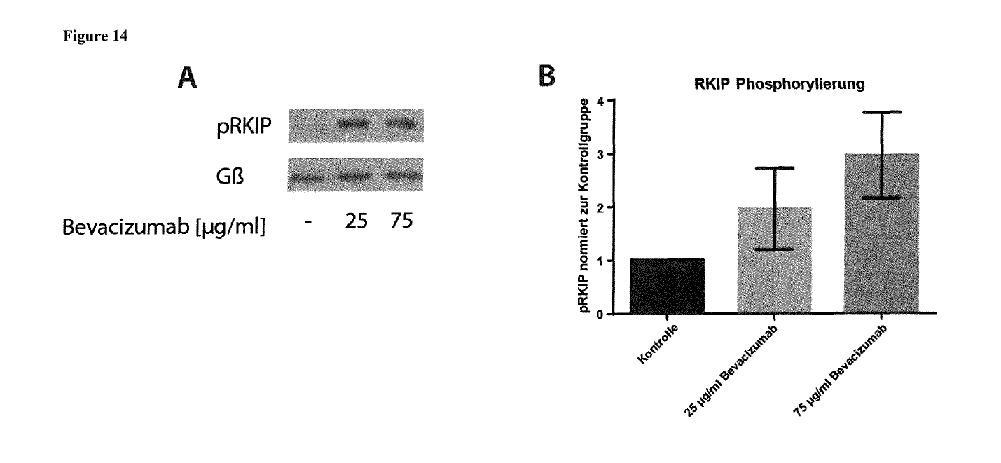

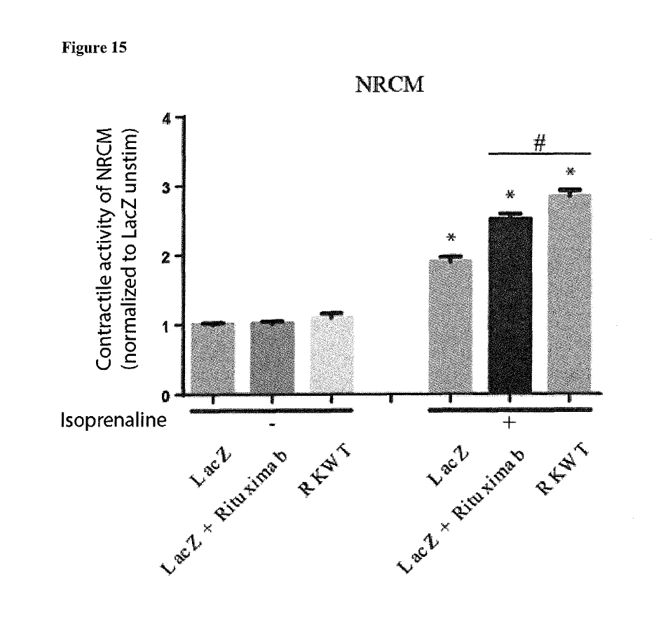

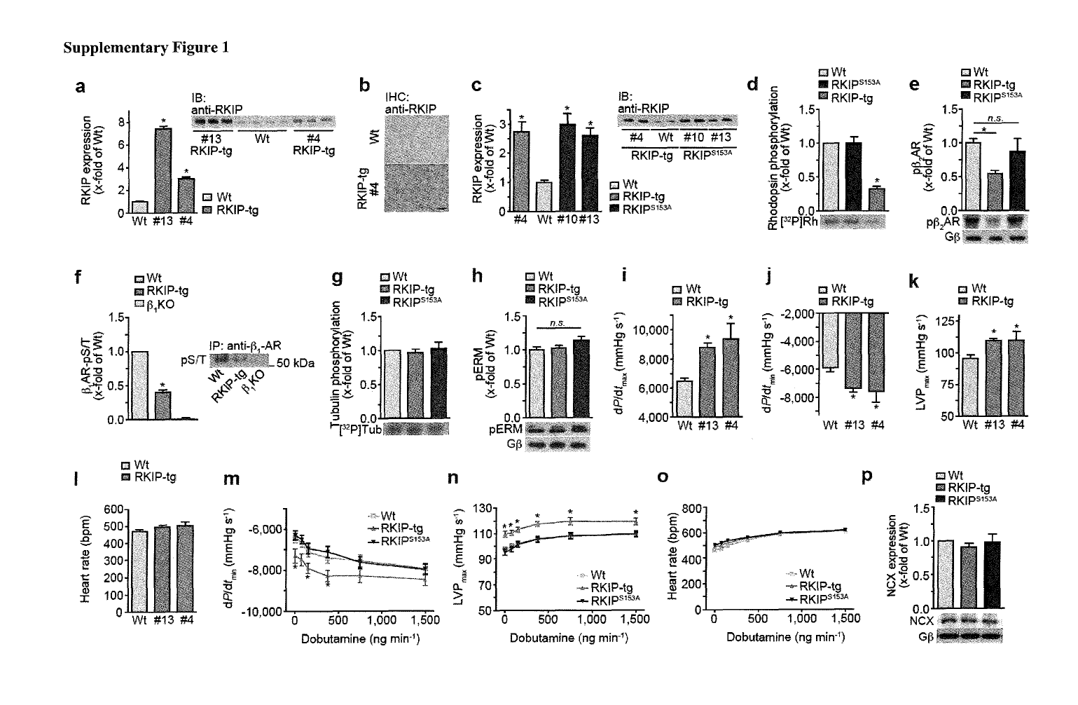

FIG. 1

RKIP Induces a Well-Tolerated Type of Chronically Increased Cardiac Contractility. Cardiac characterization of wild-type (Wt), RKIP transgenic (RKIP-tg) and RKIP^{S153A} transgenic mice at the age of 8 weeks by (a) echocardiographic fractional shortening (FS; Wt, n=14; RKIP-tg, n=11; RKIP^{S153A}, n=13) and (b) dobutamine dose response curves for dP/dtₘₐₓ determined by measurements of left ventricular pressures (Wt, n=14; RKIP-tg, n=11; RKIP^{S153A}, n=10). (c,d) Representative recordings of intracellular Ca²⁺-transients in adult cardiomyocytes isolated from indicated mice recorded with Fura-2 (340/380 nm ratio) and stimulated (c) at 0.5 Hz or (d) with 10 mM caffeine (detailed data are shown in Supplementary Tables 3 and 4). Cardiac characterization of RKIP-tg and Wt mice at the age of 12 months by (e) echocardiographic fractional shortening (FS; Wt, n=13; RKIP-tg, n=17), (f) dobutamine dose response curves for dP/dtₘₐₓ (Wt, n=10; RKIP-tg, n=12), (g) quantification of apoptosis (TUNEL-positive cardiomyocytes; Wt, n=13; RKIP-tg, n=9) and (h) analysis of fibrosis (representative Sirius Red-stained sections of left ventricular myocardium; scale bar, 20 μm). (i) Kaplan-Meier survival curves of Wt and RKIP-tg mice (n=148 mice per group). Mean±SEM; *, p<0.05 vs. Wt and RKIP^{S153A}; n represent the number of mice.

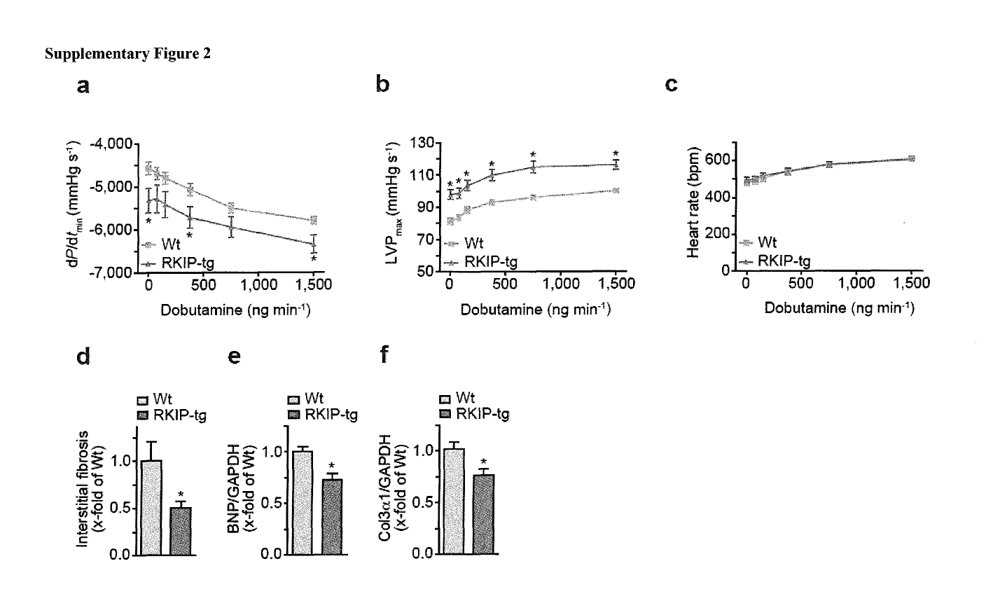

FIG. 2

RKIP Protects Mice from Pressure Overload-Induced Heart Failure. (a,b)

Immunoblot analyses of RKIP expression and phosphorylation at position 153 (pRKIP) (a) in hearts of control (Con) mice and mice after 3 weeks of pressure overload-induced by transverse aortic constriction (TAC) (n=9 mice per group) and (b) of healthy control (Con) and failing human hearts (HF) (n=7 independent samples per group). Mean±SEM; *, p<0.05 vs. Con. (c-h) Cardiac characterization of Wt, RKIP-tg and RKIP^{-/-} mice under basal conditions at the age of 8 weeks (Con) or after 3 weeks of TAC: (c) echocardiographic fractional shortening (FS; for n see Supplementary Table 6), (d) representative M-mode echocardiograms, (e) quantification of apoptosis (TUNEL-positive cardiomyocytes; controls: Wt, n=16; RKIP-tg, n=13; RKIP^{-/-}, n=8; TAC: Wt, n=14; RKIP-tg, n=8; RKIP^{-/-}, n=10; n represent the number of mice), (f) representative Sirius Red-stained left ventricular sections (scale bar, 20 μm), (g) lung weight-to-tibia length ratios (LW/TL; for n see Supplementary Table 6) and (h) Kaplan-Meier survival curves in response to TAC (Wt, n=22; RKIP-tg, n=21; RKIP^{-/-}, n=25). Mean±SEM; *, p<0.05 vs. Con; †, p<0.05 vs. Wt TAC, ‡, p<0.05 vs. Wt Con and RKIP^{-/-} Con; #, p<0.05 vs. Wt TAC and RKIP-tg TAC.

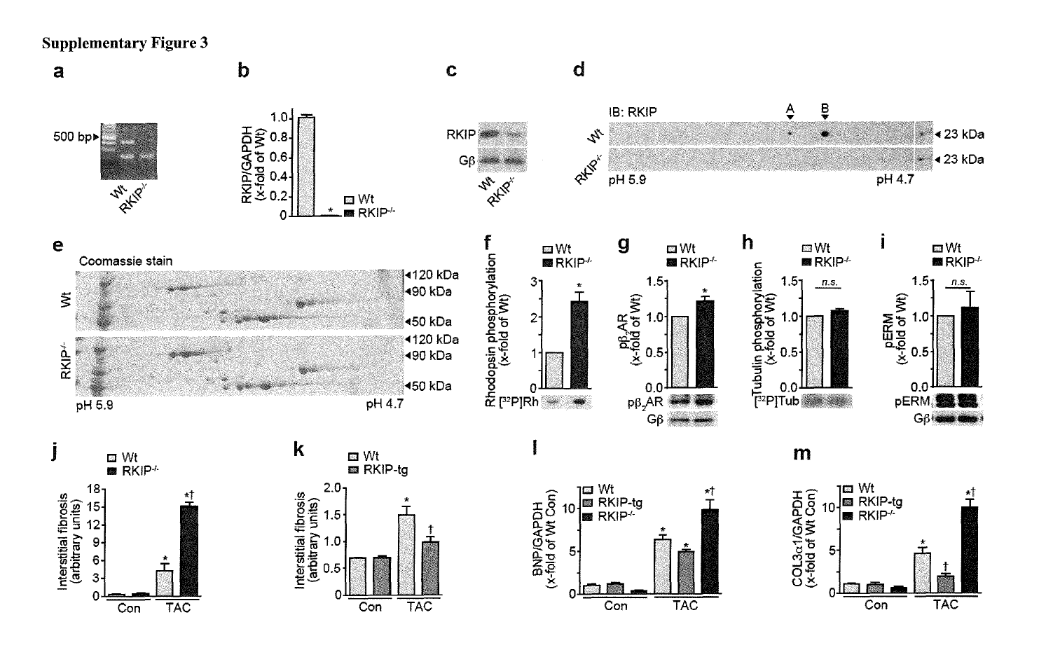

FIG. 3

AAV9-RKIP-Treated Mice are Protected from TAC-Induced Heart Failure.

Adeno-associated virus serotype 9 (AAV9) vectors encoding for RKIP under the control of the CMV-enhanced α-myosin light chain promoter were intravenously injected in (a-d) Wt and (e-h) RKIP^{-/-} mice after TAC surgery. Shown are analyses of Wt or RKIP^{-/-} mice under basal conditions at the age of 8 weeks (Con) and after 4 weeks of TAC and AAV9-GFP or AAV9-RKIP treatment: (a,e) echocardiographic fractional shortening (FS); (b,f) representative M-mode echocardiograms; (c,g) representative Sirius Red-stained transverse left ventricular sections (scale bar, 1 mm) and (d,h) lung weight-to-tibia length ratios (LW/TL). Mean±SEM; *, p<0.05 vs. Con; #, p<0.05 as indicated. For n see Supplementary Tables 7 and 8.

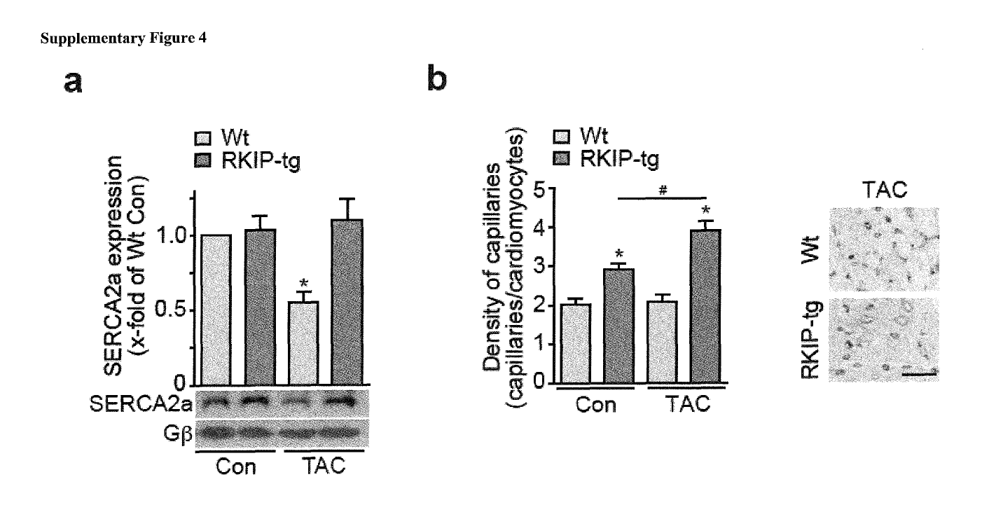

FIG. 4

RKIP Enhances β-Adrenoceptor Signaling.

Left ventricular (a) dP/dtₘₐₓ and (b) dP/dtₘᵢₙ from catheter measurements in Wt and RKIP-tg mice before and after esmolol infusion. Wt, n=7 mice; RKIP-tg, n=8 mice; mean±SEM; *, p<0.05 vs. all other conditions. (c-j) Cardiac phenotyping of β₁- and β₂-knockout mice without (β₁KO; β₂KO) or with cardiac overexpression of RKIP (RKIP-tg/β₁KO; RKIP-tg/β₂KO) under basal conditions (Con) and 6 weeks after TAC by (c,d) echocardiographic fractional shortening (FS; for n see Supplementary Tables 9 and 10); (e) representative recordings of intracellular Ca²⁺-transients in adult cardiomyocytes isolated from indicated mice recorded with Fura-2 (340/380 nm ratio) and stimulated at 0.5 Hz (detailed data are shown in Supplementary Table 11); (f,h) quantification of apoptosis (TUNEL positive cardiomyocytes; controls: β₁KO, n=9; RKIP-tg/β₁KO, n=7; β₂KO, n=7; RKIP-tg/β₂KO, n=8; TAC: β₁KO, n=12; RKIP-tg/β₁KO, n=12; β₂KO, n=9; RKIP-tg/β₂KO, n=10; and (g,i) Sirius Red-stained left ventricular sections (scale bars, 20 μm). (j) Kaplan-Meier survival curves of β₂KO and RKIP-tg/β₂KO. n=58 (β₂KO) and n=54 (RKIP-tg/β₂KO). Mean±SEM; *, p<0.05 vs. Con; †, p<0.05 vs. β₂KO; p<0.05 vs. β₁KO TAC. n represent the number of mice.

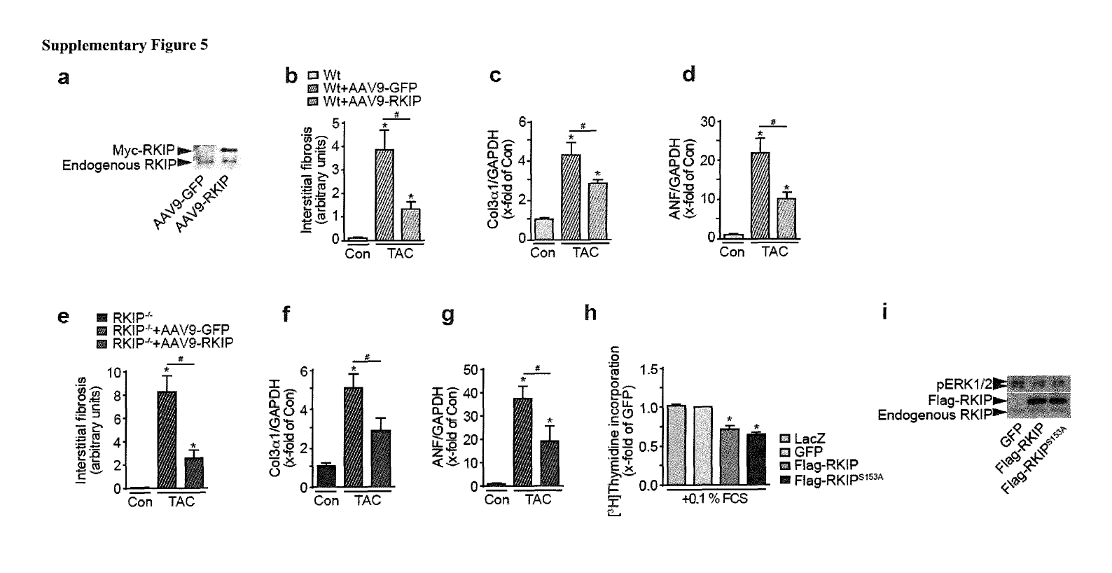

FIG. 5

β₂AR Protect RKIP-Tg Mice from Diastolic SR Ca²⁺-Leaks. (a-i)

Characterization of the calcium-handling proteins by (a, c, e, g) immunoblot analyses of phospho-phospholamban (pPLN[S16]), phospho-troponin I (pTnI[SS23/24]), phospho-ryanodine receptor 2 (pRyR2[S2808] and pRyR2[S2814]), L-type calcium channel phosphorylation (pCa_{vβ[Thr}498], a CaMKII phosphorylation site in the β₂ₐ subunit, that was detected using phospho-CaMKII(Thr286) antibodies⁵⁸, pCaᵥ1.2[Ser1928], a PKA phosphorylation site in the α_{1c} subunit, and either total TnI or Caᵥ1.2 as loading control in heart lysates of (a) Wt and RKIP-tg mice (n=5), (c) β₂KO and RKIP-tg/β₂KO mice (n=8), (e) Wt and RKIP-tg and RKIP^{S153A} mice (n=6), (g) β₂KO and RKIP-tg/β₂KO mice (n=6). (b,d) Quantification of diastolic Ca²⁺-spark frequency (confocal laser-scan, Fluo-4) and illustrative 3D-plots of Ca²⁺ in cardiomyocytes from (b) Wt and RKIP-tg and (d) β₂KO and RKIP-tg/β₂KO mice. Wt, n=73 cells of 6 mice; RKIP-tg, n=70 cells of 8 mice; β₂KO, n=52 cells of 6 mice; RKIP-tg/β₂KO, n=96 cells of 7 mice. (f, h, i) Current-voltage relationships of I_{Ca,L} (pA/pF) recorded from isolated cardiomyocytes of (f) Wt (n=28), RKIP-tg (n=28) and RKip^{S153A} (n=29) or (h) β₂KO (n=26) and RKIP-tg/β₂KO (n=22) and (i) representative recordings in β₂KO and RKIP-tg/β₂KO cardiomyocytes; mean±SEM; *, p<0.05 vs. Wt or β₂KO, respectively. (j,k) Immunoblot analysis of pAkt with Akt as loading control in heart lysates of indicated mice. n=6 mice per group; mean±SEM; *, p<0.05 vs. Wt or β₂KO, respectively. (1) Representative telemetric ECG traces of β₂KO and RKIP-tg/β₂KO mice (scale bar, 0.2 s) and (m) quantification of ectopic beats obtained by telemetric measurements before and after isoproterenol injection (Iso; 50 μg; i.p.). Lines represent mean; *, p<0.05 vs. all other conditions (ANOVA); †, p<0.05 vs. β₂KO treated without isoproterenol (t-test); n=9 mice per group.

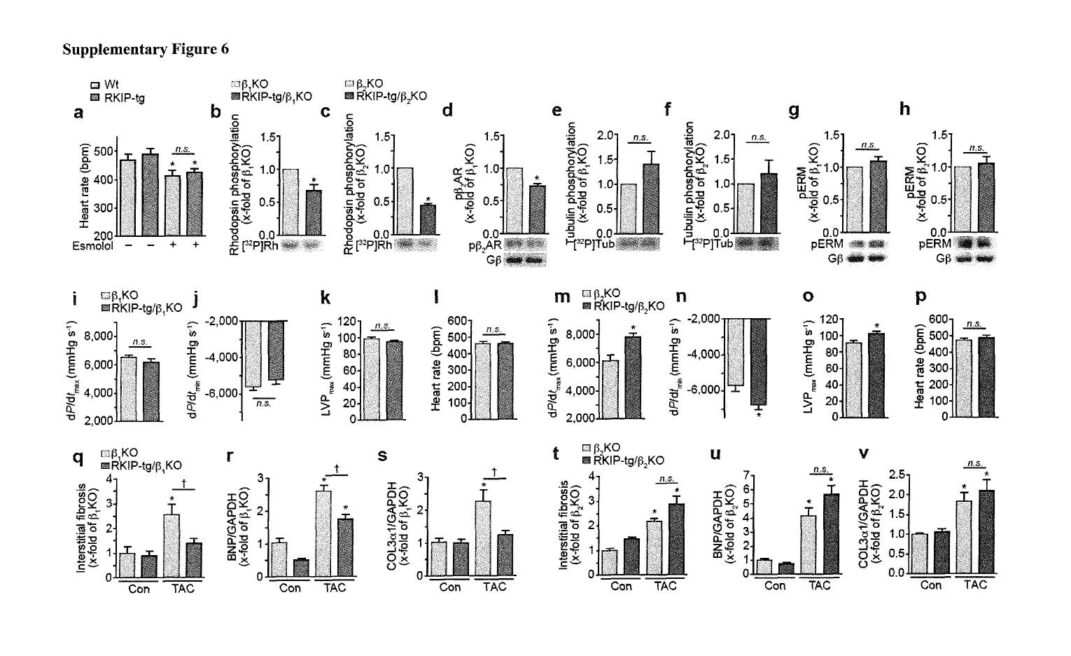

FIG. 6

RKIP Induces Positive Inotropy but Also Prevents from Apoptosis and Ectopic Beats.

Unphosphorylated RKIP inhibits Raf1 and thereby ERK1/2 activation. Upon phosphorylation at position 153 by protein kinase C (PKC) RKIP is released from Raf1; phosphorylated RKIP binds to the N-terminus of GRK2 and interferes with GRK-mediated receptor phosphorylation and subsequent receptor desensitization but does not affect phosphorylation of cytosolic substrates. RKIP mediates increased contractility and relaxation via increased β₁-adrenergic signaling (via increased phospholamban (PLN) and troponin I (TnI) phosphorylation) and anti-apoptotic, anti-fibrotic and anti-arrhythmic effects via increased β₂-adrenergic signaling. Continuous Gᵢ signaling of β₂AR prevents β₁AR-stimulated increases in ryanodine receptor 2 (RyR2) and L-type Ca²⁺ channel (LTCC) phosphorylation and triggers Akt activation effects that most likely contribute to the protective effects of RKIP.

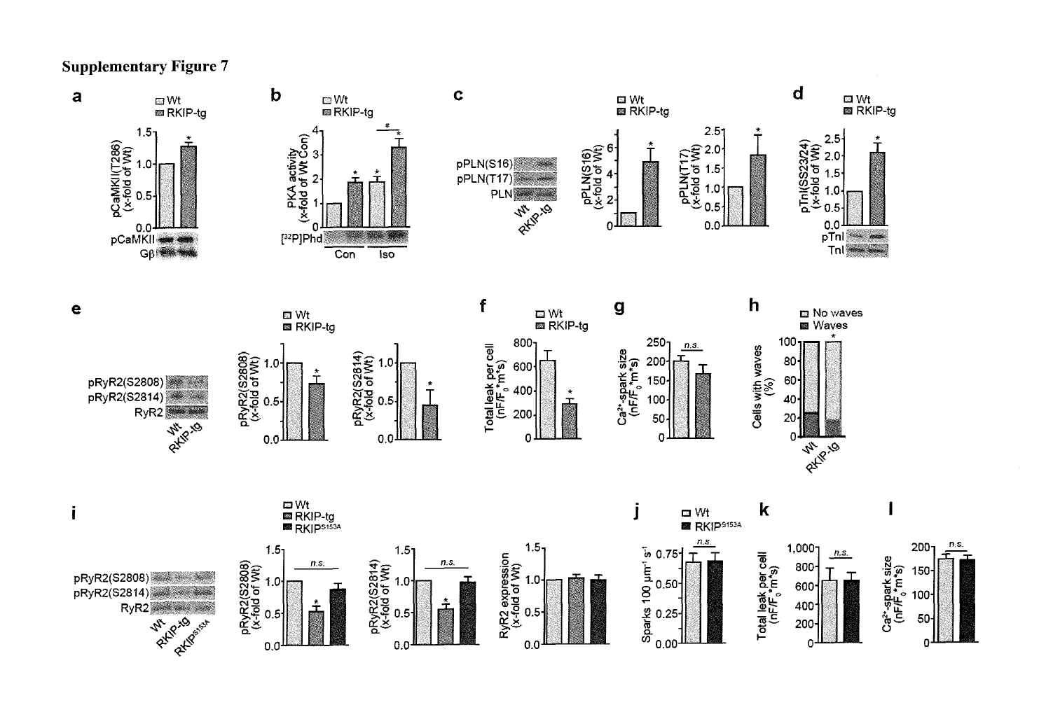

FIG. 7

Alignment of RKIP Sequences of Rat and Human

An alignment of the RKIP sequence of rat (below) and human (above) is shown.

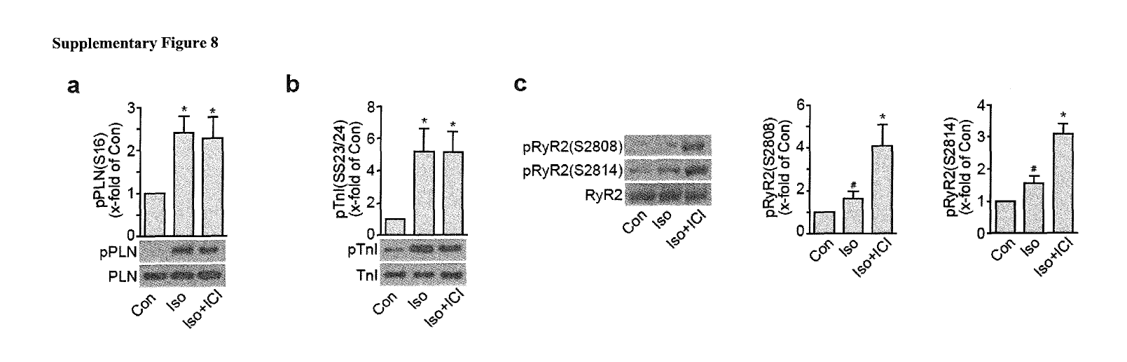

FIG. 8

Alignment of RKIP Sequences of Rat and Mouse

An alignment of the RKIP sequence of rat (below) and mouse (above) is shown.

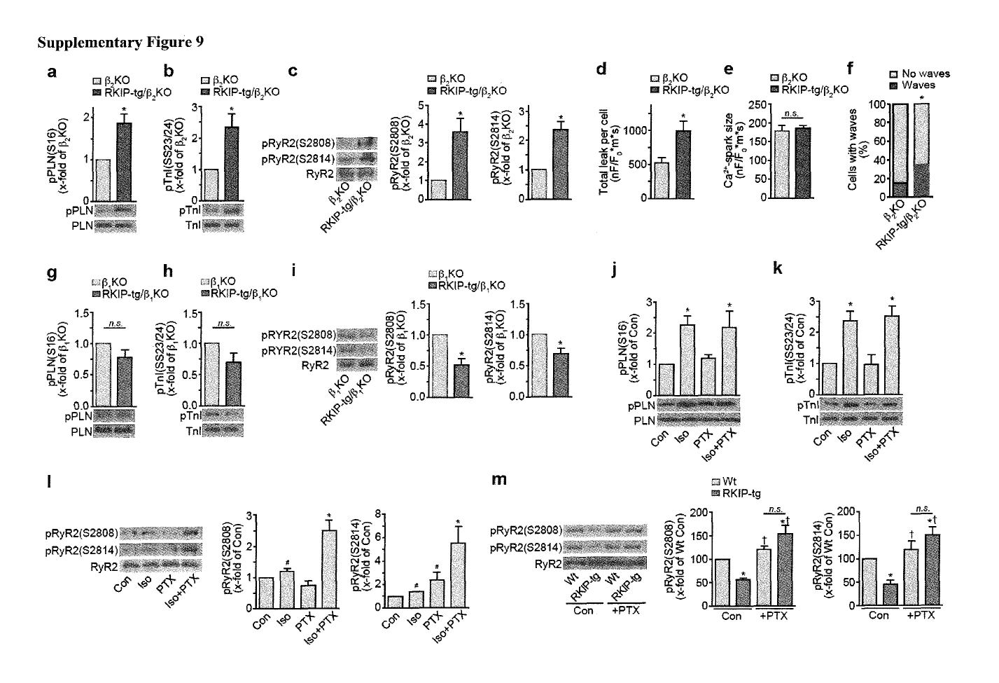

FIG. 9

Differential RKIP Expression and Phosphorylation in Patients with High- and Low-Gradient Aortic Stenosis.

Analysis of septum samples of patients with severe aortic valve stenosis (aortic valve area<1 cm²) subgrouped in patients with high (≥40 mmHg; highgradient) or low (<40 mmHg; low-gradient) transvalvular pressure gradients. Immunoblot analysis of RKIP phosphorylation (pRKIP) and RKIP expression in patients with low (n=9) and high gradients (n=16) compared with nonfailing control myocardium (n=7). Mean±SEM; *, p<0.05 vs. Con; #, p<0.05 vs. High; n represent the number of independent samples.

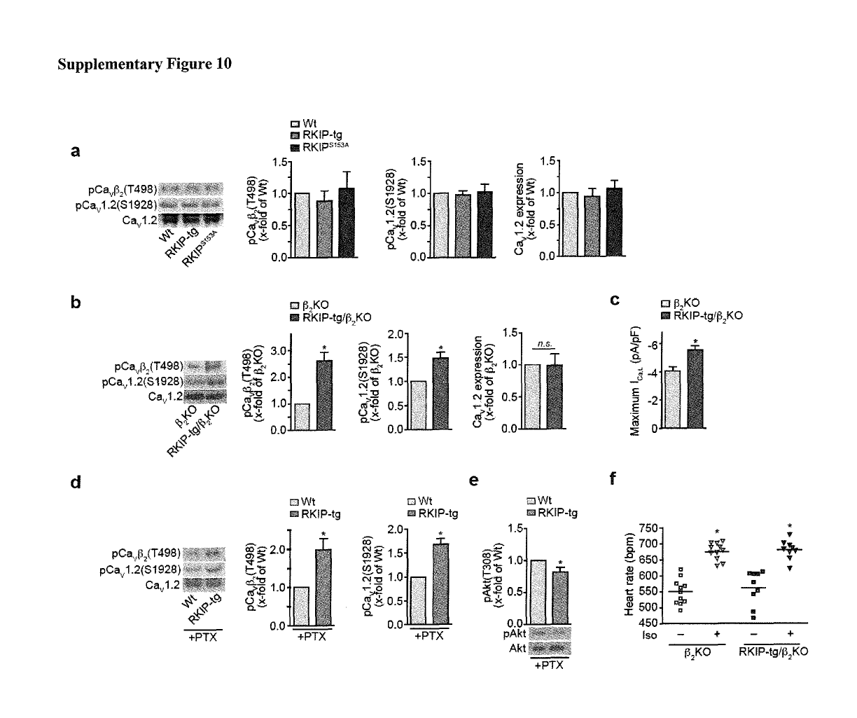

FIG. 10

RKIP Expression and RKIP Phosphorylation are Reduced in Ischemic Cardiomyopathy.

Analysis of myocardial samples of patients with ischemic cardiomyopathy (ICM) compared to nonfailing control myocardium (Con). Immunoblot analysis of RKIP phosphorylation (pRKIP) and RKIP expression in patients with ICM (n=6) and nonfailing control myocardium (n=7). Mean±SEM; *, p<0.05 vs. Con; n represent the number of independent samples.

FIG. 11

Induktion of RKIP Expression

500 000 NRCM/Well were plated in 12 Well plates, cultured for 24 h and subsequently treated with the indicated compounds for 30 h inkubiert. Cells were lysed, Western blots performed and signals were quantified using ImageJ (n=11). Shown are SEM; *. p=0.0003

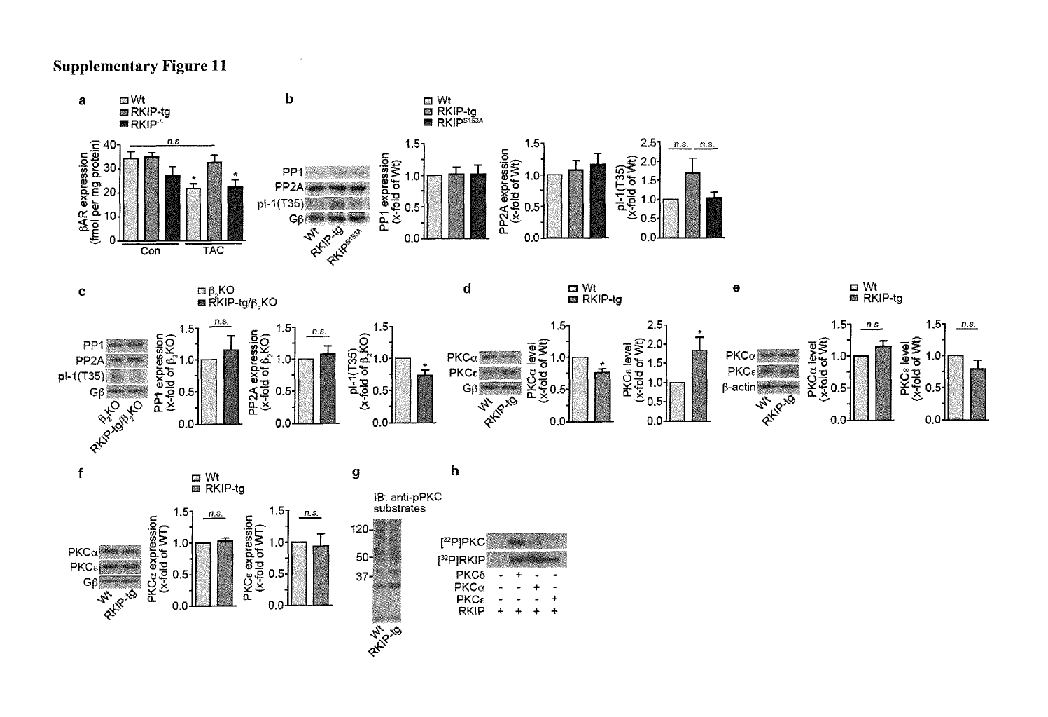

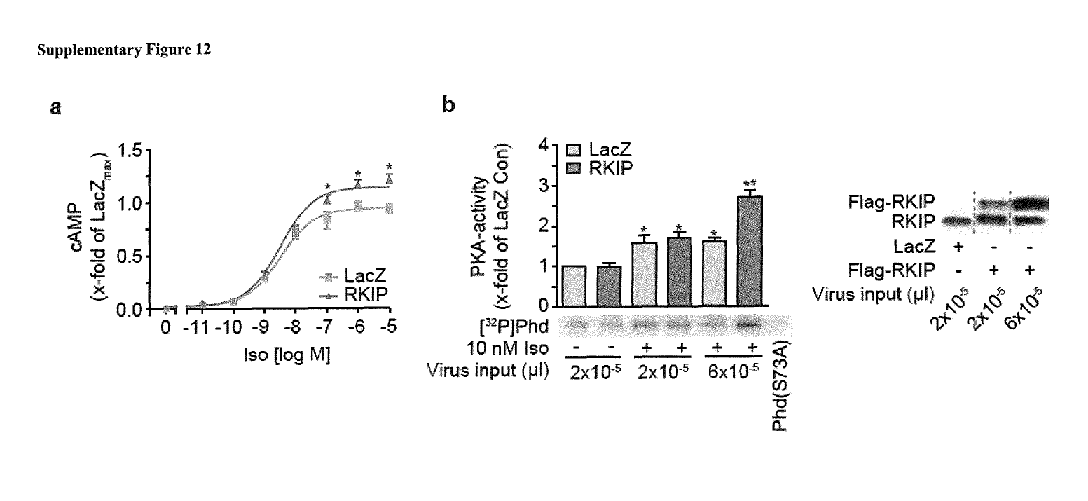

FIG. 12

Anti-CD20 Antibodies (Such as Rituximab) and Anti-VEGF Antibodies (Such as Bevacizumab) Activate RKIP (pRKIP, Ser153-Phosphorylation)

NRCM were treated with rituximab (25 μg u. 75 μg/ml) or bevacizumab (25 μg u. 75 μg/ml) for 30 h and Western blot analysis performed. Gβ was used as loading control.

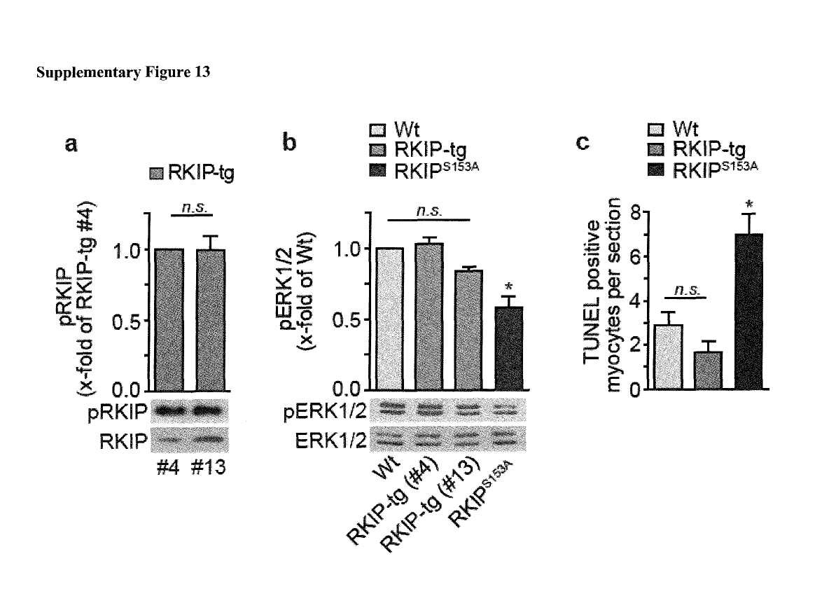

FIG. 13

Anti-CD20 Antibodies (Such as Rituximab) Activate RKIP (pRKIP, Ser153-Phosphorylation)

NRCM were treated with rituximab (25 μg u. 75 μg/ml) for 30 h and Western blot analysis and quantification using ImageJ was performed. Gβ was used as loading control. SEM, n=3, *, p=0.0004

FIG. 14

Anti-VEGF Antibodies (Such as Bevacizumab) Activate RKIP (pRKIP, Ser153-Phosphorylation)

NRCM were treated with rituximab (25 μg u. 75 μg/ml) for 30 h and Western blot analysis and quantification using ImageJ was performed. Gβ was used as loading control. SEM, n=4, *, p=0.0004

FIG. 15

Contractile Activity of NRCM in the Presence of an Anti-CD20 Antibody (Such as Rituximab) or RKIP-Wt

NRCM were cultured for 24 h and transduced as indicated with a control virus (LacZ) or a virus encoding RKIP-wt and/or treated with rituximab as indicated. The spontaneous contractions were measured at 33° C. for one minute and normalized for each experiment to LacZ under unstimulated conditions. NRCM were then stimulated with isoproterenol (50 nM) and after 2 min contractions were counted again. SEM; *, p<0.0001, n=5

Supplementary Figure Legends

Supplementary FIG. 1

Characterization of Wt, RKIP-Tg and RKIP^{S153A} Mice at the Age of 8 Weeks. (a-c)

Generation of transgenic mice overexpressing wild-type RKIP (RKIP-tg) or RKIP^{S153A} under the control of the α-myosin heavy chain (α□Mhc) promoter. Two independent founder lines were analyzed for each genotype. (a) Immunoblot analysis of RKIP expression levels in Wt, RKIP-tg line #13 and RKIP-tg line #4 with respective quantification. (b) Immunhistochemical staining (IHC) of RKIP in left ventricular sections in Wt and RKIP-tg (line #4) mice (scale bar, 20 μm). These are representative stainings of n=3 mice per group. (c) Immunoblot analysis of RKIP expression levels in RKIP^{S153A} (line #10) and RKIP^{S153A} (line #13) mice compared to expression levels in Wt and RKIP-tg (line #1) mice with respective quantification. n=4 mice per group. If not indicated otherwise, experiments within the manuscript were done with RKIP-tg line #4 and RKIP^{S153A} line #10. (d-h) Analysis of G protein-coupled receptor kinase (GRK) activity in Wt, RKIP-tg and RKIP^{S153A} mice by (d) autoradiographic detection of rhodopsin phosphorylation (Rh; Wt, n=17 mice; RKIP-tg, n=13 mice; RKIP^{S153A}, n=6 mice) and (e) immunoblot analysis of phospho-β₂AR (pβ₂AR) (n=7 mice per group); (f) immunoblot analysis of phospho-serine/threonine residues of immunoprecipitated β₁AR (β₁AR-pS/T; n=4 mice per group; β₁KO samples were included as negative controls), (g) autoradiographic detection of β-tubulin phosphorylation (Tub; n=7 mice per group) and (h) immunoblot analysis of phospho-ezrin/radixin/moesin (pERM) as cytosolic substrate of GRK (n=7 mice per group). Gβ was used as loading control. (i-o) Hemodynamic measurements of Wt, RKIP-tg (line #4 and line #13) and RKIP^{S153A} mice (line #10 and line #13) at the age of 8 weeks. (i-l) Basal measurements of maximum rate of left ventricular pressure change (dP/dtₘₐₓ), minimum rate of left ventricular pressure change (dP/dtₘₘ), maximum left ventricular pressure (LVPₘₐₓ), and heart rate; of note, hemodynamic measurements in RKIP-tg mouse lines #4 and #13 revealed similar phenotypes. Wt, n=6; RKIP-tg (line #13), n=5; RKIP-tg (line #4), n=5. (m-o) Dose response curves of dobutamine infusion for dP/dtₘᵢₙ, LVPₘₐₓ and average heart rates. Wt, n=14; RKIP-tg, n=11; RKIP^{S153A} n=10. (p) Immunoblot analysis of NCX expression levels with respective quantification (n=7). Mean±SEM; *, p<0.05 vs. Wt and RKIP^{S153A} or as indicated. n represent the number of mice.

Supplementary FIG. 2

Characterization of Wt and RKIP-Tg Mice at the Age of 12 Months. (a-c)

Hemodynamic measurements in Wt and RKIP-tg mice at the age of 12 months. Dose response curves of dobutamine infusion for minimum rate of left ventricular pressure change (dP/dtₘᵢₙ), maximum left ventricular pressure (LVPₘₐₓ) and average heart rates (Wt, n=10; RKIP-tg, n=12). (d) Quantification of interstitial fibrosis in Sirius Red-stained heart sections of Wt and RKIP-tg mice at the age of 12 months (Wt, n=15; RKIP-tg, n=16; n represent the analysis of one transverse section per mice). (e,f) mRNA expression of brain natriuretic peptide (BNP; Wt, n=10; RKIP-tg, n=11) and collagen type III alpha 1 (COL3α1; Wt, n=10; RKIP-tg, n=8) normalized to glycerinaldehyde-3-phosphate dehydrogenase (GAPDH). n represent numbers of mice measured in triplicates. Mean±SEM; *, p<0.05 vs. Wt.

Supplementary FIG. 3

Characterization of RKIP^{-/-} and RKIP-Tg Mice in Response to Chronic Pressure Overload. (a-e)

Verification of complete RKIP deletion in RKIP^{-/-} mice. (a) PCR genotyping of homozygous RKIP^{-/-} mice using a primer pair annealing around the gene trap vector insertion site; upper band represents an amplified RKIP sequence, the lower band represents the amplification of a myosin sequence as PCR control. (b) Detection and quantification of RKIP mRNA expression levels by real-time PCR in Wt (n=4) and RKIP^{-/-} (n=5) mice. Detection of RKIP protein deletion in RKIP^{-/-} mice by (c) Western blot analysis (repeated 3 times with similar results) and (d) additional 2D-SDS gel electrophoresis revealed that the remaining anti-RKIP antibody signal in RKIP^{-/-} mice is an unspecific band. For isoelectric focusing a narrow pH range (pH4.7-pH5.9) was chosen to exclude largely unspecific proteins. The right panel depicts a non-focused part of the immobilized pH gradient strip and, thus, only one-dimensional separation of the respective heart lysates. Spot “A” in the left panel represents unphosphorylated RKIP, while spot “B” represents phosphorylated RKIP. The experiment was repeated 3 times with similar results. (e) Corresponding Coomassie staining of the upper region of the 2D-SDS gels of (d) as loading control. (f-i) Analysis of G protein-coupled receptor kinase (GRK) activity in heart lysates of Wt and RKIP^{-/-} by (f) autoradiographic detection of rhodopsin phosphorylation (n=3 mice per group), (g) immunoblot analysis of phospho-β₂AR (pβ₂AR; n=4 mice per group) as well as (h) autoradiographic detection of β-tubulin phosphorylation (n=6 mice per group) and (i) immunoblot analysis of phospho-ezrin/radixin/moesin (pERM; n=4 mice per group) as cytosolic substrates of GRK. Gβ was used as loading control. (j,k) Quantification of interstitial fibrosis of Sirius Red-stained left ventricular sections under control conditions (Con) and after 3 weeks of transverse aortic constriction (TAC) of Wt, RKIP-tg and RKIP^{-/-} mice. Controls: Wt, n=5 and 7; RKIP-tg, n=10; RKIP^{-/-}, n=4; TAC: Wt, n=7 and 16; RKIP-tg, n=12; RKIP^{-/-}, n=5; n represent the analysis of one transverse section per mice. (l,m) Quantification of brain natriuretic peptide (BNP; controls: Wt, n=6; RKIP-tg, n=5; RKIP^{-/-}, n=4; TAC: Wt, n=5; RKIP-tg, n=3; RKIP^{-/-}, n=8; n represent numbers of mice measured in triplicates) and collagen type III alpha 1 (COL3α1; controls: Wt, n=7; RKIP-tg, n=4; RKIP^{-/-}, n=4; TAC: Wt, n=6; RKIP-tg, n=5; RKIP^{-/-}, n=6; n represent numbers of mice measured in triplicates) mRNA expression levels in Wt, RKIP-tg and RKIP^{-/-} mice (normalization for GAPDH mRNA). Mean±SEM; *, p<0.05 vs. controls (Con) or Wt, respectively; †, p<0.05 vs. Wt TAC.

Supplementary FIG. 4

Analysis of SERCA2a Expression and Capillary-Myocyte Density in Response to TAC. (a)

Immunoblot analysis of SERCA2a expression levels in control mice and mice after 3 weeks of TAC. Mean±SEM; *, p<0.05 vs. all other conditions; n=8 mice per group. Gβ expression was used as loading control. (b) Determination of the number of capillaries per cardiomyocytes in a total of 12 randomized visual fields of left ventricular myocardium per mouse (image magnification 400×). Representative immunohistochemical staining of endothelial cells using anti-CD31 antibodies. Scale bar, 50 μm; mean±SEM; *, p<0.05 vs. Wt Con; #, p<0.05 as indicated; Wt Con, n=10; Wt TAC, n=9; RKIP-tg Con, n=14; RKIP-tg TAC, n=14; n represent the number of mice.

Supplementary FIG. 5

Analysis of AAV9-RKIP-Treated Wt and RKIP^{-/-} Mice that had Undergone TAC Surgery. (a)

Immunoblot showing myc-RKIP expression in the heart of Wt mice 4 weeks after AAV9-GFP or AAV9-RKIP injection. (b-d) Analysis of AAV9-treated Wt mice: (b) quantification of interstitial fibrosis by the analysis of Sirius Red-stained left ventricular sections of Wt mice under control conditions (Con) and mice after 4 weeks of transverse aortic constriction (TAC) and AAV9-GFP or AAV9-RKIP treatment. Mean±SEM; *, p<0.05 vs. Con; #, p<0.05 as indicated; n=10 mice per group, i.e. analysis of one transverse section per mouse. (c,d) Quantification of collagen type III alpha 1 (c; COL3α1; n=9) and atrial natriuretic factor (d; ANF; n=9) mRNA levels in Wt mice under control conditions (Con) and mice after 4 weeks of TAC and AAV9-GFP or AAV9-RKIP treatment (normalization for GAPDH mRNA). n represent numbers of mice measured in triplicates; mean±SEM; *, p<0.05 vs. Con. #, p<0.05 as indicated. (e-g) Analysis of AAV9-treated Wt mice: (e) quantification of interstitial fibrosis of Sirius Red-stained left ventricular sections of RKIP^{-/-} mice under control conditions (Con) and mice after 4 weeks of TAC and AAV9-GFP or AAV9-RKIP treatment. Mean±SEM; *, p<0.05 vs. Con; #, p<0.05 as indicated; n=10 mice per group, i.e. analysis of one transverse section per mice. (f,g) Quantification of COL3α1 (f; n=9) and ANF (g; n=9) mRNA levels in RKIP^{-/-} mice under control conditions (Con) and mice after 4 weeks of TAC and AAV9-GFP or AAV9-RKIP treatment (normalization for GAPDH mRNA). n represent numbers of mice measured in triplicates; mean±SEM; *, p<0.05 vs. Con; #, p<0.05 as indicated. (h,i) Effects of RKIP on proliferation in neonatal rat fibroblasts. (h) [³H]Thymidine incorporation experiment in cells adenovirally transduced with Flag-tagged RKIP (Flag-RKIP) or RKIP^{S153A} (Flag-RKIP^{S153A}) in the presence of 0.1% (v/v) FCS for 96 h. As controls fibroblasts transduced with β-galactosidase (LacZ) or green fluorescent protein (GFP) were used. Mean±SEM; *, p<0.05 vs. LacZ or GFP; n=12; n represent independent experiments of 4 different fibroblast preparations. (i) Representative immunoblots showing RKIP overexpression and ERK1/2 phosphorylation (pERK1/2) in neonatal rat fibroblasts transduced with indicated adenoviruses. n=3 independent experiments.

Supplementary FIG. 6

Characterization of βAR Signaling in RKIP-Tg.

(a) Average heart rate of cardiac catheterization experiments of FIG. 4 in wild-type (Wt; n=7) and RKIP transgenic (RKIP-tg; n=8) mice before and after esmolol infusion (*, p<0.05 vs. unstimulated Wt and RKIP-tg mice). (b-h) Analysis of G protein-coupled receptor kinase (GRK) activity in heart lysates of RKIP-tg lacking β₁- or β₂AR (RKIP-tg/β₁KO or RKIP-tg/β₂KO) compared to β₁- or β₂KO mice, respectively. (b,c) Autoradiographic detection of rhodopsin phosphorylation (Rh; b, RKIP-tg/β₁KO vs. β₁KO, n=6 mice per group; c, RKIP-tg/β₂KO vs. β₂KO, n=5 mice per group), (d) immunoblot analysis of phospho-β₂AR (pβ₂AR; RKIP-tg/β₁KO vs. β₁KO, n=5 mice per group) and (e,f) autoradiographic detection of β-tubulin phosphorylation (Tub; e, RKIP-tg/β₁KO vs. β₁KO, n=5 mice per group; f, RKIP-tg/β₂KO vs. β₂KO, n=6 mice per group) and (g,h) immunoblot analysis of phospho-ezrin/radixin/moesin (pERM; g, RKIP-tg/β₁KO vs. β₁KO, n=5 mice per group; h, RKIP-tg/β₂KO vs. β₂KO, n=6 mice per group) as cytosolic substrates of GRK. Gβ was used as loading control. (i-p) Determination of (i,m) dP/dtₘₐₓ and (j,n) dP/dtₘᵢₙ, (k,o) maximum left ventricular pressure (LVPₘₐₓ) and (l,p) average heart rate determined by measurements of left ventricular pressures in β₁- and β₂KO mice without (β₁KO; β₂KO) or with cardiac overexpression of RKIP ([i-l] RKIP-tg/β₁KO; [m-p] RKIP-tg/β₂KO). β₁KO, n=10; RKIP-tg/β₁KO, n=10; β₂KO, n=7; RKIP-tg/β₂KO, n=8; mean±SEM; *, p<0.05 vs. β₂KO. (q-v) Quantification of interstitial fibrosis, brain natriuretic peptide (BNP) and collagen type III alpha 1 (COL3α1) mRNA levels in (q-s) β₁KO and RKIP-tg/β₁KO and (t-v) β₂KO and RKIP-tg/β₂KO mice. (q,t) Interstitial fibrosis was determined by analysis of left ventricular Sirius Red-stained heart sections. Controls: β₁KO, n=5; RKIP-tg/β₁KO, n=5; β₂KO, n=5; RKIP-tg/β₂KO, n=7; TAC: β₁KO, n=11; RKIP-tg/β₁KO, n=10; β₂KO, n=6; RKIP-tg/β₂KO, n=9; n represent the analysis of one transverse section per mouse. (r,u) BNP and (s,v) COL3α1 expression determined by real-time PCR analysis normalized to GAPDH mRNA. Controls: β₁KO, n=5; RKIP-tg/β₁KO, n=4; β₂KO, n=4; RKIP-tg/β₂KO, n=6; TAC: β₁KO, n=6; RKIP-tg/β₁KO, n=5; β₂KO, n=6; RKIP-tg/β₂KO, n=10; n represent numbers of mice measured in triplicates. Mean±SEM; *, p<0.05 vs. Con; †, p<0.05 as indicated.

Supplementary FIG. 7

RKIP Protects from Diastolic SR Ca²⁺-Leak. (a,b)

Analysis of CaMKII and PKA activity in adult cardiomyocytes isolated from Wt and RKIP-tg mice by (a) immunoblot analysis of pCaMKII(T286) with Gβ as loading control (n=6 mice per group) and (b) autoradiographic detection of phosducin (Phd) phosphorylation in the presence of isoproterenol (Iso). Phd was used as an in vitro substrate for PKA. n=4 cardiomyocyte isolations; mean±SEM; *, p<0.05 vs. Con or Wt, respectively; #, p<0.05 as indicated. (c-e) Immunoblot quantification of phospho-phospholamban (c; pPLN[S16], n=14 mice per group; and pPLN[T17], n=8 mice per group), phospho-troponin I (d; pTnI[SS23/24]; n=9 mice per group), phospho-ryanodine receptor 2 (e; pRyR2[S2808] and pRyR2[S2814]; n=17 mice per group) in Wt and RKIP-tg. (f) Total diastolic sarcoplasmatic reticulum (SR) Ca²⁺-leak (Wt, n=73 cells of 6 mice; RKIP-tg, n=70 cells of 8 mice), (g) Ca²⁺-spark size (Wt, n=238 sparks of n=73 cells of 6 mice; RKIP-tg, n=121 sparks of n=70 cells of 8 mice) and (h) the percentage of cardiomyocytes with spontaneous Ca²⁺-waves (Wt, n=324 cells of 6 mice; RKIP-tg, n=380 cells of 8 mice) of all cardiomyocytes isolated from Wt and RKIP-tg investigated by confocal laser scan microscopy. (i) Immunoblot quantification of RyR2 phosphorylations (pRyR2[S2808] and pRyR2[S2808]; n=7 mice per group) and of RyR2 in heart lysates of Wt, RKIP-tg and RKIP^{S153A} mice. (j) Quantification of diastolic Ca²⁺-spark frequency (confocal laser-scan, Fluo-4) and (k) total diastolic SR Ca²⁺-leak in cardiomyocytes isolated from Wt and RKIP^{S153A} mice. Mean±SEM; Wt, n=97 cells of 6 mice; RKIP^{S153A}, n=99 cells of 6 mice. (l) Ca²⁺-spark size (Wt, n=363 sparks of n=97 cells of 7 mice; RKIP^{S153A}, n=375 sparks of n=99 cells of 7 mice). Mean±SEM; *, p<0.05 vs. Wt and RKIP^{S153A}.

Supplementary FIG. 8

β₂AR Blockade with ICI 118,551 Promotes Isoproterenol-Induced RyR2 Phosphorylation. (a-c)

Immunoblot quantification of (a) pPLN(S16), (b) pTnI (SS23/24), (c) pRyR2(S2808) and pRyR2(S2814) in heart lysates of untreated (Con) and isoproterenol-treated mice and mice treated with Iso in combination with ICI 118,551. pPLN(S16), n=13; pTnI(SS23/24), n=11; pRyR2(S2808), n=12; n are mice per group; mean±SEM; *, p<0.05 vs. Con. Immunoblot analysis of Con vs. Iso for RyR2 phosphorylation (pRyR2[S2808] and pRyR2[S2814]) show significant differences when a t-test analysis is applied (#, p<0.05 vs. Con).

Supplementary FIG. 9

β₂AR-G₁ Signaling Protects from RyR2 Hyperphosphorylation and Diastolic SR Ca²⁺-Leak. (a-c)

Immunoblot quantification of (a) pPLN(Ser16), (b) pTnI(SS23/24), (c) pRyR2(S2808) and pRyR2(S2814) in heart lysates of β₂KO and RKIP-tg/β₂KO (pPLN[Ser16], n=5; pTnI[SS23/24], n=10; pRyR2[S2808], n=13; n are mice per group). (d) Total diastolic SR Ca²⁺-leak (β₂KO, n=52 cells of 6 mice; RKIP-tg/β₂KO, n=96 cells of 7 mice), (e) Ca²⁺-spark size (β₂KO, n=152 sparks of n=52 cells of 6 mice; RKIP-tg/β₂KO, n=509 sparks of n=96 cells of 7 mice) and (1) the percentage of cardiomyocytes with spontaneous Ca²⁺-waves (β₂KO, n=311 cells of 6 mice; RKIP-tg, n=309 cells of 8 mice) of cardiomyocytes isolated from β₂KO and RKIP-tg/β₂KO. (g-i) Immunoblot quantification of (g) pPLN(S16), (h) pTnI(SS23/24), (i) pRyR2(S2808) and pRyR2(S2814) in heart lysates of β₁KO and RKIP-tg/β₁KO (pPLN[S16], n=6; pTnI[SS23/24], n=6; pRyR2[S2808], n=7; pRyR2[S2814], n=7; n are mice per group). (j-l) Immunoblot analyses of (j) pPLN(S16), (k) pTnI(SS23/24) and (l) pRyR2(S2808 and S2814) in heart lysates of Wt mice that were untreated, treated with Iso or pertussis toxin (PTX; 300 μg/kg; i.p.; 24 h) alone or treated with the combination of Iso and PTX (Iso+PTX). pPLN(S16), n=5; pTnI(SS23/24), n=5; pRyR2(S2808) and pRyR(S2814), n=7; n are mice per group. Mean±SEM; *, p<0.05 vs. β₂KO, β₁KO or Con, respectively. Immunoblot analysis of Con vs. Iso for RyR2 phosphorylation (pRyR2[S2808] and pRyR2[S2814]) show significant differences when a t-test analysis is applied (#, p<0.05 vs. Con). (m) Immunoblot analyses of pRyR2(S2808 and 52814) in heart lysates of Wt and RKIP-tg mice that were untreated or treated with pertussis toxin (PTX; 300 μg/kg; i.p.; 24 h). n=7 mice per group; mean±SEM; *, p<0.05 vs. Wt Con; †, p<0.05 vs. RKIP-tg Con.

Supplementary FIG. 10

Expression and Phosphorylation of LTCC are not Altered by RKIP-Tg or RKIP^{S153A} Overexpression. (a,b)

Immunoblot analysis of the L-type Ca²⁺ channel (LTCC) by determination of LTCC phosphorylation at threonine 498, a CaMKII phosphorylation site in the β₂ₐ subunit, that was detected using phospho-CaMKIIδ(T286) antibodies⁵⁸ (pCaᵥβ₂[T498]), LTCC phosphorylation at serine 1928, a PKA phosphorylation site in the α_{1c} subunit (pCaᵥ1.2[S1928]) and Caᵥ1.2 expression levels in heart lysates of (a) Wt, RKIP-tg and RKIP^{8153A} mice (n=6 mice per group) and (b) β₂KO and RKIP-tg/β₂KO mice (n=6 mice per group). (c) Effects of RKIP on peak I_{Ca,L} (Vₘ+10 mV) in mice that lack β₂AR. (d,e) Immunoblot analysis of (d) pCaᵥβ₂[T498], pCaᵥ1.2[S1928] and (e) pAkt with respective loading controls in hearts of indicated mice after treatment with pertussis toxin (PTX; 300 μg/kg, 24 h). n=6 mice per group. (f) Heart rates of telemetric ECG measurements in FIG. 5m of β₂KO (n=11) and RKIP-tg/β₂KO (n=9) before and after Iso injection (i.p.) as indicated (lines represent mean). Mean±SEM; *, p<0.05 vs. Wt or β₂KO under basal conditions.

Supplementary FIG. 11

Further Characterization of Downstream Signaling in Wt, RKIP-Tg, RKIP^{S153A}, β₂KO and RKIP-Tg-β₂KO Mice.

(a) β-adrenergic receptor (βAR) density in Wt, RKIP-tg and RKIP^{-/-} mice under control conditions (Con) and after 3 weeks of transverse aortic constriction (TAC). Controls: Wt, n=6; RKIP-tg, n=4; RKIP^{-/-}, n=5; TAC: Wt, n=5; RKIP-tg, n=4; RKIP^{-/-}, n=4; n represent numbers of mice measured in duplicates; mean±SEM; *, p<0.05 vs. Wt Con. (b,c) Immunoblot analysis of protein phosphatase 1 and 2A (PP1 and PP2A), phosphorylation of phosphatase inhibitor 1 (0-1) with GP as loading control in heart lysates of (b) Wt, RKIP-tg and RKIP^{8153A} mice (n=7 mice per group, except for n=12 for pI-1 in RKIP-tg) and (c) β₂KO and RKIP-tg/β₂KO mice (n=6 mice per group). Mean±SEM; *, p<0.05 vs. β₂KO. (d-f) Immunoblot analyses of protein kinase C isoform α and ε (PKCα and PKCε) in (d) membrane or (e) cytosolic fractions of Wt and RKIP-tg hearts or of (f) unfractionated heart lysates with Gβ or β-actin as loading controls. n=5 mouse hearts per group; mean±SEM; *, p<0.05 vs. Wt. (g) Immunoblot analyses of phosphorylated PKC targets using phospho-PKC substrate antibodies in Wt and RKIP-tg mice. Shown is a representative Western blot of n=6 mice per group. (h) In vitro RKIP phosphorylation by PKCα, PKCδ and PKCε. Shown is a representative experiment showing the autoradiographies of PKC autophosphorylation and RKIP phosphorylation of n=4 independent experiments. Mean±SEM; *, p<0.05 vs. Wt and RKIP-tg.

Supplementary FIG. 12

The Potency of RKIP to Sensitize β-Adrenergic Signaling Depends on RKIP Expression Levels. (a)

cAMP concentration response curves of isoproterenol in neonatal rat cardiomyocytes (NRCM) transduced with control (LacZ) or RKIP viruses (2×10⁻⁵ μl). Mean±SEM; maximum isoproterenol response of LacZ was normalized to 1; n=12 per group; a non-linear curve fit was applied; *, p<0.05 vs. LacZ. (b) Purified phosducin (Phd) was used as a PKA substrate to detect PKA activity in lysates of NRCM that were adenovirally transduced with control (LacZ) or different amounts of RKIP virus (2×10⁻⁵ or 6×10⁻⁵ μl) and treated with isoproterenol (10 nM, 10 min). PKA activity was determined by autoradiographic detection of phosducin phosphorylation. Samples containing an unphosphorylatable mutant of phosducin (Phd^{S73A}) served as control. The immunoblot (right) shows the expression levels of endogenous RKIP and exogenous Flag-tagged RKIP under the conditions indicated. n=4 experiments; *, p<0.05 vs. unstimulated controls; #, p<0.05 vs. all other conditions.

Supplementary FIG. 13

Characterization of ERK1/2 Signaling in RKIP-Tg and RKIP^{S153A} Mice. (a,b)

Immunoblot analysis of (a) RKIP and pRKIP (n=5 mice per group) and (b) pERK1/2 (n=4 mice per group) in heart lysates of Wt, RKIP-tg line #4 and #13 and RKIP^{S153A} as indicated. RKIP expression levels in line #4 and #13 are shown for comparison in (a) and ERK1/2 as loading control in (b). Mean±SEM; *, p<0.05 vs. Wt and RKIP-tg. Comment to (a,b): in fact, the amounts of phosphorylated RKIP were the same in both mouse lines indicating increased amounts of unphosphorylated RKIP. In line with this, phosphorylation of ERK1/2 was undistinguishable from wild-type mice upon ˜3-fold RKIP overexpression, while a slight reduction of ERK1/2 phosphorylation was seen upon ˜8-fold RKIP overexpression. Significant ERK1/2 inhibition by unphosphorylated RKIP was verified in mice transgenic for the phosphorylation-deficient Raf1-inhibitory RKIP^{S153A} mutant. (c) Quantification of TUNEL-positive cardiomyocytes of left ventricular myocardium of indicated mice at the age of 8 weeks. Wt, n=16; RKIP-tg, n=15; RKIP^{S153A}, n=8; mean±SEM; *, p<0.05 vs. Wt and RKIP-tg.

Supplementary Table 1

Basic Echocardiographic Characterization of Wt, RKIP-Tg and RKIP^{S153A} Mice at the Age of 8 Weeks.

Echocardiographic analyses of wild-type (Wt), RKIP transgenic (RKIP-tg; line #4) and RKIP^{S153A} transgenic (RKIP^{S153A}; line #10 and line #13) mice. Shown are measurements of wall thicknesses (interventricular septal [IVS] and left ventricular posterior wall [LVPW]), left ventricular internal dimension (LVID), fractional shortening (FS), ejection fraction (EF) and heart rates. Values represent mean (±SEM); *, p<0.05 vs. all other conditions.

Supplementary Table 2

Echocardiographic Characterization of Wt and RKIP-Tg Mice after Chronic Isoproterenol Infusion.

Echocardiographic measurements and histological analysis of interstitial fibrosis of wild-type (Wt) and RKIP transgenic (RKIP-tg) mice before and after 4 weeks of isoproterenol application via osmotic minipumps (Iso; 30 mg/kg/d). Shown are measurements of wall thicknesses (interventricular septal [IVS] and left ventricular posterior wall [LVPW]), left ventricular internal dimension (LVID), fractional shortening (FS), ejection fraction (EF), heart rates and the quantification of interstitial fibrosis (arbitrary units) of Sirius Red-stained left ventricular myocyrdial sections. Values represent mean (±SEM); *, p<0.05 vs. Con; #, p<0.05 vs. Wt Iso; $, p<0.05 vs. Wt Con. n represent the number of mice (for the analysis of interstitial fibrosis one transverse section was analyzed per mouse).

Supplementary Table 3

Ca²⁺-Transient and Contraction Analyses of Cardiomyocytes Isolated from Wt, RKIP-Tg and RKIP^{S153A} Mice.

Measurements of intracellular Ca²⁺-transients (Fura-2; 340/380 nm ratio) and myocyte contraction (sarcomere shortening) at 0.5 Hz stimulation in adult cardiomyocytes isolated from Wt, RKIP-tg and RKIP^{S153A} mice. We evaluated relaxation time (time to baseline 50% and 90%), amplitude of transients (peak height) and % of sarcomere shortening. Values represent mean (±SEM); *, p<0.05 vs. Wt and #, p<0.05 vs. RKIP^{S153A}.

Supplementary Table 4

Caffeine-Induced Ca²⁺-Transients of Cardiomyocytes Isolated from Wt, RKIP-Tg and RKIP^{S153A} Mice.

Measurements of caffeine-induced intracellular Ca²⁺-transients measured with Fura-2 (340/380 nm ratio) in adult cardiomyocytes isolated from Wt, RKIP-tg and RKIP^{S153A} mice. We evaluated the amplitudes of Ca²⁺-transients at 0.5 Hz and in response to caffeine as a measure of SR Ca²⁺-content and the Ca²⁺-transient decay kinetics (Tₑₐ, monoexponential time constant of transient decay) as indirect measure of NCX function. Values represent mean (±SEM); *, p<0.05 vs. Wt and RKIP^{S153A}.

Supplementary Table 5

Echocardiographic Characterization of Wt, RKIP-Tg and RKIP^{S153A} Mice at the Age of 12 Months.

Echocardiographic analyses of Wt, RKIP-tg and RKIP^{S153A} mice. Shown are measurements of wall thicknesses (interventricular septal [IVS] and left ventricular posterior wall [LVPW]), left ventricular internal dimension (LVID), fractional shortening (FS), ejection fraction (EF) and heart rates. Values represent mean (±SEM); *, p<0.05 vs. all other genotypes.

Supplementary Table 6

Characterization of Wt, RKIP-Tg and RKIP^{-/-} Mice in Response to Pressure Overload.

Echocardiographic analyses of Wt, RKIP-tg and RKIP^{-/-} mice at the age of 8 weeks (Con) or after 3 weeks of pressure overload-induced by transverse aortic constriction (TAC) as indicated. Shown are measurements of wall thicknesses (interventricular septal [IVS] and left ventricular posterior wall [LVPW]), left ventricular internal dimension (LVID), fractional shortening (FS), ejection fraction (EF), heart rates, pressure gradients after TAC and heart weight-to-tibia length ratios (HW/TL). Values represent mean (±SEM); *, p<0.05 vs. respective control; †, p<0.05 vs. Wt and RKIP-tg after TAC; ‡, p<0.05 vs. Wt and RKIP^{-/-} after TAC; §, p<0.05 vs. Wt Con and RKIP^{-/-} Con.

Supplementary Table 7

Characterization of Wt Mice in Response to TAC and AAV9-Mediated Gene Transfer of RKIP.

Echocardiographic analyses of Wt mice at the age of 8 weeks (Con) or after 4 weeks of pressure overload-induced by transverse aortic constriction (TAC) and intravenously injected adeno-associated virus serotype 9 (AAV9)-RKIP vectors or AAV9-GFP (green fluorescent protein) as control vectors. Shown are measurements of wall thicknesses (interventricular septal [IVS] and left ventricular posterior wall [LVPW]), left ventricular internal dimension (LVID), fractional shortening (FS) and ejection fraction (EF), heart rates, pressure gradients after TAC and heart weight-to-tibia length ratios (HW/TL). Values represent mean (±SEM); *, p<0.05 vs. Wt Con; #, p<0.05 vs. Wt+AAV9-RKIP after TAC.

Supplementary Table 8

Characterization of RKIP^{-/-} Mice in Response to TAC and AAV9-Mediated Gene Transfer of RKIP.

Echocardiographic analyses of RKIP^{-/-} mice at the age of 8 weeks (Con) or after 4 weeks of pressure overload-induced by transverse aortic constriction (TAC) and intravenously injected adeno-associated vectors serotype 9 (AAV9)-RKIP or AAV9-GFP (green fluorescent protein) as control vectors. Shown are measurements of wall thicknesses (interventricular septal [WS] and left ventricular posterior wall [LVPW]), left ventricular internal dimension (LVID), fractional shortening (FS) and ejection fraction (EF), heart rates, pressure gradients after TAC and heart weight-to-tibia length ratios (HW/TL). Values represent mean (±SEM); *, p<0.05 vs. RKIP^{-/-}; #, p<0.05 vs. RKIP^{-/-}+AAV9-RKIP after TAC.

Supplementary Table 9

Characterization of β₁KO and RKIP-Tg/B₁KO in Response to TAC.

Echocardiographic analyses of mice lacking β₁AR (β₁KO) and of RKIP transgenic mice crossed into the β₁KO background (RKIP-tg/(β₁KO) at the age of 8 weeks (Con) or after 6 weeks of pressure overload-induced by transverse aortic constriction (TAC) as indicated. Shown are measurements of wall thicknesses (interventricular septal [IVS] and left ventricular posterior wall [LVPW]), left ventricular internal dimension (LVID), fractional shortening (FS) and ejection fraction (EF), heart rates, pressure gradients after TAC and heart weight-to-tibia length ratios (HW/TL). Values represent mean (±SEM); *, p<0.05 vs. control conditions; †, p<0.05 vs. β₁KO after TAC; ‡, p<0.05 vs. β₁KO Con.

Supplementary Table 10

Characterization of (3₂KO and RKIP-Tg/β₂KO in Response to TAC.

Echocardiographic analyses of mice lacking β₂AR ((3₂KO) and of RKIP transgenic mice crossed into the 13₂KO background (RKIP-tg/β₂KO) at the age of 8 weeks (Con) or after 6 weeks of pressure overload-induced by transverse aortic constriction (TAC) as indicated. Shown are measurements of wall thicknesses (interventricular septal [WS] and left ventricular posterior wall [LVPW]), left ventricular internal dimension (LVID), fractional shortening (FS) and ejection fraction (EF), heart rates, pressure gradients after TAC and heart weight-to-tibia length ratios (HW/TL). Values represent mean (±SEM); *, p<0.05 vs. control conditions; #, p<0.05 vs. β₂KO after TAC; §, p<0.05 vs. β₂KO Con; $, p<0.05 vs. RKIP-tg/β₂KO Con.

Supplementary Table 11

Ca²⁺-Transient and Myocyte Contraction Analyses of Cardiomyocytes Isolated from β₁KO, RKIP-Tg/(β₁KO, β₂KO and RKIP-Tg/β₂KO Mice

Analyses of intracellular Ca²⁺-transients measured with Fura-2 (340/380 nm ratio) and of myocyte contractions as determined by sarcomere shortening at 0.5 Hz stimulation in adult cardiomyocytes isolated from β₁- and β₂AR-knockout mice without β₁KO; β₂KO) or with cardiac overexpression of RKIP (RKIP-tg/β₁KO; RKIP-tg/β₂KO). We evaluated relaxation time (time to baseline 50% and 90%), amplitude of transients (peak height) and % of sarcomere shortening. Values represent mean (±SEM); *, p<0.05 vs. respective controls.

Supplementary Table 12

Clinical Data

Clinical data in patients with severe AS indicate that high gradient AS is associated with better myocardial function than low gradient AS

The Examples illustrate the invention.

### EXAMPLE 1: CARDIAC RKIP CAUSES A BENEFICIAL β-ADRENOCEPTOR-DEPENDENT POSITIVE INOTROPY

In heart failure therapy, it is generally assumed that attempts to produce long-term increases of cardiac contractile force are almost always accompanied by structural and functional damage.

Here we show that modest overexpression of the Raf kinase inhibitor protein (RKIP) produces a well-tolerated, persistently increased cardiac contractility, mediated via β₁-adrenoceptors (β₁AR). This is surprising since β₁AR are major drivers of cardiac contractility, but usually also of long-term adverse effects. RKIP achieves this surprising tolerance via simultaneous persistent activation of the β₂AR-subtype. Analogously, deletion of RKIP exaggerates pressure overload-induced cardiac failure. Increased expression of RKIP in murine and human heart failure further suggests that RKIP is one of the body's strategies to maintain cardiac function. Positive effects of RKIP gene transfer in a murine heart failure model illustrate new therapeutic options in heart failure therapy.

Results

RKIP Induces Persistent Positive Inotropy

To investigate the role of RKIP in the heart in vivo, we generated transgenic mice with cardiac-specific expression of wild-type RKIP (RKIP-tg) or of RKIP^{S153A}—a RKIP mutant, which cannot be phosphorylated by PKC and therefore is not able to bind and inhibit GRK (Supplementary FIG. 1a-c)^{15,17}. In vitro rhodopsin phosphorylation experiments with heart lysates and immunoblot analyses of β₂AR phosphorylation verified that GRK activity was significantly reduced in mouse hearts of RKIP-tg but not of RKIP^{S153A} mice; likewise β₁AR serine/threonine phosphorylations were impaired in RKIP-tg mouse heart lysates (Supplementary FIG. 1d-f). However, cytosolic GRK substrate phosphorylation was not affected by RKIP overexpression as shown by maintained in vitro β-tubulin phosphorylation and immunoblot analysis of ezrin/radixinimoesin phosphorylation (Supplementary FIG. 1g,h).

Echocardiography and left ventricular catheterization revealed positive inotropic and lusitropic effects in RKIP-tg mice compared to Wt or RKIP^{S153A} mice (FIG. 1a,b, Supplementary FIG. 1-o and Supplementary Table 1): cardiac fractional shortening as well as velocities of contraction and relaxation were significantly elevated in RKIP-tg mice. Of note, infusion of the β-adrenergic agonist dobutamine during left ventricular catheterization showed that RKIP- and dobutamine-induced increases in contractility and relaxation are almost additive. RKIP-induced inotropic effects were also additive with those caused by chronic isoproterenol treatment (Supplementary Table 2). Interestingly, RKIP-induced increases in basal contractility and relaxation as well as increases in contractility during chronic isoproterenol are different from the GRK2-knockout (GRK2-KO) phenotype: Cardiac-specific GRK2-KO mice show positive inotropy rather in response to acute β-adrenergic stimulation, but display adverse remodeling and a decline in contractility in response to chronic β-adrenergic stimulation^{9,10,14}.

Single cell experiments with cardiomyocytes isolated from Wt, RKIP-tg and RKIP^{S153A} mice further substantiated the positive effects of RKIP on contractility and relaxation. Cardiomyocytes isolated from RKIP-tg mice had faster kinetics of Ca²⁺-reuptake into the sarcoplasmatic reticulum (SR), higher Ca²⁺-transient amplitudes and a faster relaxation. Further, SR Ca²⁺-load was enhanced as assessed by caffeine-induced SR Ca²⁺-release (FIG. 1c,d and Supplementary Tables 3 and 4). Of note, similar decay kinetics of caffeine-induced Ca²⁺-transients and Western blots suggest that NCX function and expression are not altered by RKIP (Supplementary FIG. 1p).

Chronic elevation of cardiac contractility is normally accompanied by impairments in myocardial structure and function (see above; Ref.^{1,2,6,19}) However, the positive inotropic phenotype of RKIP-tg persisted at least up to the age of 12 months (FIG. 1e-f, Supplementary FIG. 2a-c and Supplementary Table 5). The histological appearance of the hearts was even better for RKIP-tg mice, i.e. they showed less cardiomyocyte apoptosis, interstitial fibrosis, BNP and collagen III expression than Wt mice, and finally the life-span of RKIP-tg was at least as long as that of Wt mice (FIG. 1g-i, Supplementary FIG. 2d-f).

Thus, our analyses of RKIP-tg mice suggest that it is indeed possible to chronically increase cardiac contractility without adverse effects on the heart.

RKIP Protects from Pressure Overload-Induced Heart Failure

To gain insight into the role of endogenous RKIP in the heart, we analyzed expression levels of RKIP in cardiac stress situations. We found that RKIP expression was upregulated in mice after chronic pressure overload induced by transverse aortic constriction (TAC) (FIG. 2a). Similar increases were found in myocardial samples of heart failure patients (FIG. 2b). Concomitantly, levels of phosphorylated and thus “GRK-inhibitory” RKIP (pRKIP) were increased. This upregulation of RKIP might indicate a potentially protective role of endogenous RKIP in heart failure.

The hypothesized beneficial and contractility enhancing role of endogenous RKIP was confirmed by functional analysis of RKIP knockout mice (RKIP^{-/-}) (Supplementary FIG. 3a-e). Of note, GRK activity towards receptor substrates of GRK (but not cytosolic ones) in heart lysates of RKIP^{-/-} mice was increased (Supplementary FIG. 3f-i)—analogously to decreased GRK activity in RKIP-tg mice, which was likewise selective towards receptor substrates.

Under basal conditions, cardiac contractility of RKIP^{-/-} mice was not different from Wt mice (FIG. 2c and Supplementary Table 6), but upon pressure overload, RKIP^{-/-} mice showed significantly more cardiac deterioration: echocardiographic fractional shortening was lower and left ventricular dilation more pronounced in RKIP^{-/-} than in Wt mice, and cardiomyocyte apoptosis, interstitial fibrosis as well as marker gene mRNA expression of BNP and collagen III were significantly higher in RKIP^{-/-} mice. The prominent increase in lung weight in RKIP^{-/-} mice indicated pulmonary congestion and exaggerated left ventricular heart failure (FIG. 2c-g, Supplementary Table 6 and Supplementary FIG. 3j,l,m). In line with this phenotype, the survival of RKIP^{-/-} mice upon pressure overload was clearly reduced (FIG. 2h).

In contrast, RKIP overexpression protected from consequences of pressure overload: cardiac contractility of RKIP-tg was hardly harmed by pressure overload and there were no significant increases in cardiomyocyte apoptosis or interstitial fibrosis, and only minor increases in BNP and collagen III expression in RKIP-tg mice (FIG. 2c-g and Supplementary FIG. 3k-m). Of note, while the extent of cardiac hypertrophy was comparable in RKIP-tg and Wt mice, cardiac hypertrophy in RKIP-tg was hardly associated with adverse effects and e.g. capillary density was even increased compared to Wt mice (for further informations refer to Supplementary FIG. 4 and Supplementary Table 6)²⁰.

Taken together, our data suggest that loss of endogenous RKIP makes the heart more susceptible to cardiac stress and exaggerates the development of congestive heart failure while overexpression of RKIP produces both increased contractility and protection from pressure overload stress. The upregulation of endogenous RKIP expression and RKIP phosphorylation upon cardiac stress seem to be part of beneficial compensatory mechanisms in the heart.

AAV9-Mediated Gene Transfer of RKIP Protects from Heart Failure

To test the potential of RKIP for heart failure therapy, we evaluated whether adeno-associated virus serotype 9 vector (AAV9)-mediated gene transfer of RKIP in mice can protect from the development of pressure overload-induced heart failure²¹. We intravenously injected AAV9 vectors encoding for RKIP. AAV9-RKIP treatment resulted in significant protection of Wt and RKIP^{-/-} mice from cardiac deterioration. Fractional shortening and left ventricular dilation, interstitial fibrosis, marker gene expression and pulmonary congestion were all improved compared to respective control mice treated with control vectors (AAV9-GFP) (FIG. 3, Supplementary FIG. 5a-g and Supplementary Tables 7 and 8). AAV9-RKIP almost completely rescued the detrimental phenotype of RKIP^{-/-} mice in response to TAC indicating that restoring RKIP in cardiomyocytes is sufficient for its beneficial effects. In line with these findings, we observed only a minor but significant anti-proliferative effect of RKIP in neonatal rat cardiac fibroblasts (Supplementary FIG. 5h,i). These experiments substantiate the therapeutic potential of RKIP.

RKIP Enhances βAR Signaling

βAR are the most efficient physiological triggers for inotropy. Therefore, RKIP might mediate its hypercontractile phenotype at least in part by increasing βAR activity via its GRK-inhibitory effects. To test this hypothesis, we infused Wt and RKIP-tg mice with the β₁-selective antagonist esmolol during left ventricular catheterization. Esmolol effectively reduced heart rates of Wt and RKIP-tg mice (Supplementary FIG. 6a) and largely attenuated increased rates of contraction and relaxation in RKIP-tg (FIG. 4a,b).

To further investigate the roles of β₁- and β₂AR for RKIP signaling, we crossed RKIP-tg mice with β₁AR knockout (β₁KO) and β₂AR knockout (β₂KO) mice to generate RKIP-tg/β₁KO and RKIP-tg/β₂KO animals^{22,23}. As in the wild-type background, GRK activity was decreased in RKIP-tg mice in the respective β₁- and/or β₂KO background, as shown by reduced receptor phosphorylation (but not cytosolic targets) indicating that RKIP can potentially increase signaling by the remaining β₁AR or β₂AR, respectively (Supplementary FIG. 6b-d).

Indeed, we found that positive inotropic and lusitropic effects of RKIP on fractional shortening and rates of cardiac contraction and relaxation were fully abolished in RKIP-tg/β₁KO animals (FIG. 4c, Supplementary FIG. 6i-1 and Supplementary Table 9). In contrast, a lack of β₂AR did not interfere with increased contractility of RKIP-tg (FIG. 4d, Supplementary FIG. 6m-p and Supplementary Table 10). Similarly, single isolated cardiomyocytes from RKIP-tg mice that lack β₁- (but not those that lack β₂-) AR lost the hypercontractile phenotype as shown by the analysis of Ca²⁺-transients and cardiomyocyte contraction (FIG. 4e and Supplementary Table 11). These findings underline that the hypercontractile phenotype of RKIP-tg is indeed largely mediated by β₁AR.

In contrast to the positive inotropic effects, the anti-apoptotic and anti-fibrotic effects of RKIP in response to chronic pressure overload were preserved in RKIP-tg/β₁KO (FIG. 4f,g and Supplementary FIG. 6q-s). Instead, RKIP-tg mice lacking β₂AR (RKIP-tg/β₂KO) developed apoptosis, interstitial fibrosis and increases in BNP and collagen III mRNA expression to a similar extent as control mice (β₂KO) (FIG. 4h,i and Supplementary FIG. 6t-v). Accordingly, transgenic RKIP was not able to prevent deterioration of cardiac function in the absence of β₂AR (Supplementary Table 10). The contention that β₂AR (but not β₁AR) are essential for the tolerance of positive inotropy in RKIP-tg mice, is further supported by an overall reduced survival rate of aging RKIP-tg/β₂KO mice compared to β₂KO mice (FIG. 4j).

### Differential Regulation of β₁AR Targets by RKIP

β₁AR are generally coupled to Gₛ proteins and initiate the generation of cAMP and activation of protein kinase A (PKA)⁶. PKA phosphorylates key regulators of contraction and relaxation such as phospholamban (PLN), troponin I (TnI), L-type Ca²⁺ channels (LTCC) and ryanodine receptors 2 (RyR2). These PKA-mediated phosphorylation events enhance Ca²⁺-cycling and reduce myofilament Ca²⁺-sensitivity in cardiomyocytes, leading to increases in force and rates of contraction and of relaxation. Ca²⁺ in turn is important for the formation of the Ca²⁺/calmodulin complex, which activates Ca²⁺/calmodulin-dependent protein kinase II (CaMKII)^{6,19}.

If hypercontractility in RKIP-tg mice is mediated via chronic activation of β₁AR, then the activity of the β₁AR downstream kinases PKA and CaMKII should be increased^{6,13,19}. Immunoblot and kinase assays showed that this was indeed the case (Supplementary FIG. 7a,b). Since chronic activation of these kinases usually triggers cardiac deterioration, RKIP-tg mice have to involve mechanisms that secure the tolerability of chronic β-adrenergic activation. We thus investigated the involvement of different β-adrenergic downstream targets in RKIP-tg mice.

In line with elevated β-adrenergic signaling and contractility, RKIP-tg mice displayed an increase in PLN and TnI phosphorylation; phosphorylation of the SR Ca²⁺-release channel RyR2, however, at both tested sites was significantly reduced in RKIP-tg mice (FIG. 5a and Supplementary FIG. 7c-e) despite higher overall activities of PKA and CaMKII (see above). Hyperphosphorylation of RyR2 by PKA and in particular by CaMKII has been reported to induce diastolic SR Ca²⁺-leaks and to contribute to the development of heart failure and arrhythmias^{13,19,24-32}. Quantification of Ca²⁺-sparks and Ca²⁺-waves revealed a lower diastolic Ca²⁺-leak in cardiomyocytes of RKIP-tg compared to Wt mice as well as a lower Ca²⁺-spark and Ca²⁺-wave frequency. The individual size of Ca²⁺-sparks was not altered (FIG. 5b and Supplementary FIG. 7f-h). Of note, phosphorylation-deficient RKIP^{S153A}, that does not affect GRK activity or receptor phosphorylation, did not affect RyR2 phosphorylations or spontaneous diastolic SR Ca²⁺-leak, supporting the view that these effects in RKIP-tg are indeed receptor-mediated (Supplementary FIG. 7i-l).

β₂AR Signaling has a Central Role in RKIP-Mediated Protective Effects

Since β₂AR seem to play an important role for the protective phenotype of RKIP-tg, we sought to examine how (3₂AR may exert their beneficial effects using pharmacologically selective stimulation or blockade of β₁AR vs. β₂AR. With regard to RyR2(S2808/S2814) phosphorylation we found that stimulation of β₁AR by isoproterenol plus simultaneous β₂AR blockage via ICI 118,551 led to a much stronger phosphorylation of RyR2 than non-selective β₁/β₂AR-stimulation. Of interest, the β₂AR blockade hardly affected PLN and TnI phosphorylation (Supplementary FIG. 8a-c). Similarly, PLN and TnI phosphorylation were increased in RKIP-tg lacking the β₂AR (RKIP-tg/β₂KO vs. β₂KO) to a similar extent as in wild-type background. The absence of β₂AR in RKIP-tg mice, however, led to a significant increase of RyR2 phosphorylations as well as Ca²⁺-sparks and -waves (RKIP-tg/β₂KO vs. β₂KO; FIG. 5c,d and Supplementary FIG. 9a-f). Analogous analyses in the absence of β₁AR showed reduced RyR2 phosphorylations in RKIP-tg mice (Supplementary FIG. 9g-i), thereby substantiating our hypothesis that β₂AR are central for RKIP-mediated protection from RyR2-hyperphosphorylation (S2808/S2814) and dysregulated Ca²⁺-cycling.

It has been reported that in contrast to β₁AR-mediated ubiquitous signaling, β₂AR and their signaling in cardiac myocytes is concentrated to the t-tubular region³³⁻³⁵ and that β₂AR can switch from stimulatory (Gₛ) to inhibitory G proteins (Gᵢ)^{36,37}. Considering the close proximity of β₂AR and RyR2 in the t-tubular region, we speculated that such a β₂AR-Gᵢ switch might explain why—in contrast to the other β₁AR targets—RyR2 was not hyperphosphorylated in RKIP-tg. To examine this proposed mechanism, we inactivated Gαᵢ in Wt mice by intraperitoneal application of pertussis toxin (PTX). This treatment resulted in a marked increase of isoproterenol-induced RyR2 phosphorylation at 52808 and 52814 but did not affect phosphorylation of PLN and TnI (Supplementary FIG. 9j-l), strongly suggesting that β₂AR-Gᵢ coupling dampens RyR2 phosphorylation. The absence of RyR2 hypo-phosphorylation in PTX-treated RKIP-tg mice (Supplementary FIG. 9m) further supported the idea that RKIP favors Gᵢ coupling of β₂AR, and thereby protects from RyR2 hyperphosphorylation and diastolic Ca²⁺-leak events that have been associated with cardiac deterioration and arrhythmias^{19,24-32}. Persistent Gᵢ-coupled β₂AR signaling may be particularly well tolerated in RKIP-tg mice in terms of cardiac contractility because of concomitant β₁AR activation.

To test whether the L-type Ca²⁺ channel (LTCC), another t-tubular target of PKA and CaMKII, is similarly affected by RKIP as the RyR2, we analyzed LTCC phosphorylation at the residues serine 1928 (PKA-site in the Caᵥ1.2 subunit) and threonine 498 (CaMKII-site in the β₂ₐ subunit) as well as L-type Ca²⁺ currents. These experiments revealed that RKIP—even though it activates β₁AR does not alter LTCC phosphorylation (FIG. 5e and Supplementary FIG. 10a) or L-type Ca²⁺ currents as shown by the evaluation of the current-voltage relationship (FIG. 5f). Analogous experiments in RKIP-tg mice lacking the β₂AR indicate that β₂AR contribute an inhibitory component to LTCC phosphorylation and current in RKIP-tg mice, since LTCC phosphorylation and L-type Ca²⁺ currents in RKIP-tg lacking the β₂AR were significantly increased compared to the respective control mice (RKIP-tg/β₂KO vs. β₂KO) (FIG. 5g-i and Supplementary FIG. 10a,c). Immunoblot analyses of LTCC phosphorylation in Wt and RKIP-tg mice further suggest that these effects are Gᵢ-dependent (Supplementary FIG. 10d).

Further, the β₂AR-Gᵢ-Akt signaling pathway has previously been identified as a pathway that mediates cell survival^{38,39}. In line with RKIP's anti-apoptotic and anti-fibrotic effects, Akt activation was significantly increased in RKIP-tg and dependent on ρ₂AR-Gᵢ-mediated signaling (FIG. 5j,k and Supplementary FIG. 10e). Thus, it is likely that Akt activation contributes to the well-tolerated positive inotropy and the protective effects of RKIP on cardiac remodeling.

Altogether, a plethora of experiments points towards a central role of concomitant β₂AR activation in RKIP-tg mice, i.e. β₂AR (i) restrain increased β₁AR signaling with regard to selective βAR downstream targets, in particular those within the t-tubular region known to participate in pro-arrhythmic effects, such as RyR2 and LTCC and (ii) activate the pro-survival pathway Akt. Interestingly, in the absence of β₂AR, RKIP-tg mice were not protected from β₁-adrenergic arrhythmic effects as shown by increased spontaneous mortality (FIG. 4j) and an enhanced number of ectopic beats compared to β₂KO control mice. This effect was even more prominent in response to isoproterenol (FIG. 5l,m and Supplementary FIG. 10f).

### DISCUSSION

Long-term stimulation of the failing heart's contractility has proven to be problematic, and many such approaches have turned out to be fraught with detrimental effects, e.g. arrhythmias or structural damage. New experimental strategies have been added in recent years with increased sarco-/endoplasmatic reticulum Ca²⁺-ATPase (SERCA2a) activity by phospholamban knockdown^{13,40}, SERCA2a overexpression^{13,41-43}, overexpression of S100A²¹ or βARK-ct induced disinhibition of LTCC¹¹. However, it is not yet understood, why some of those strategies are promising, while others rather accelerate cardiac deterioration like chronic (i) activation of βARs by isoproterenol or dobutamine^{1,2}, (ii) inhibition of phosphodiesterase III¹, (iii) PKA activation⁴⁴, (iv) inhibition of protein phosphatase 1⁴⁵, or (v) CaMKII activation^{19,32}.

Our study on RKIP in the heart provides a new and well-tolerated inotropic strategy and at the same time an explanation for these apparent discrepancies: the mode of βAR activation seems to be decisive for protective vs. detrimental β-adrenergic stimulation. The protective effects of increased RKIP expression are due to continuous and “balanced” activation of both β₁AR and β₂AR, that (i) stimulates β₁-Gₛ-signaling, which enhances contractility primarily via increased SERCA2a activation, TnI phosphorylation and SR Ca²⁺-load; (ii) protects, however, other β-adrenergic downstream targets from activation despite continuous β₁AR activation; this effect appears to be due to β₂AR signaling and to affect particularly targets within the t-tubular region³³⁻³⁵ as seen from a lack of PKA- and CaMKII-mediated phosphorylation of RyR2 and LTCC; dysregulation of RyR2 and LTCC are triggers for arrhythmia and the development of heart failure^{19,24-32,47}; and (iii) stimulates the kinase Akt that is known to mediate anti-apoptotic effects^{38,39}. Presumably, the effects of RKIP are of more permanent nature than direct βAR stimulation since RKIP inhibits βAR desensitization and downregulation (Supplementary FIG. 11a). Continuous β₁AR-Gₛ-PKA activation even seems to assure the PKA-mediated switch of β₂AR from Gₛ to Gᵢ that appears crucial for the characteristic phosphorylation pattern of βAR downstream targets in RKIP-tg mice. This pattern is most likely caused by the differential activation of the respective kinases, since we found no evidence for alterations in phosphatase activity (Supplementary FIG. 11b-c). Even though many of our mechanistic studies are based on βAR-KO mice, that might evolve compensatory mechanisms^{22,48,49}, the acute use of specific β₁AR antagonists—a different and cleaner model—agrees that β₁AR signaling is required for the positive inotropic effects of RKIP overexpression, while β₂AR signaling is mandatory for the cardioprotective effects.

PKC activity is increased in heart failure and known to accelerate cardiac hypertrophy and failure^{50,51}. RKIP might well represent a protective feedback mechanism for excessive PKC activation since RKIP is upregulated in heart failure and since PKC enables RKIP to act as a GRK inhibitor^{15,50,51}. Moreover, PKC isoforms activated in RKIP-tg mice appear to involve favorable PKCs, i.e. PKCε, that—unlike PKCα—are associated with cardioprotective effects (Supplementary FIG. 11d-h)⁵⁰⁻⁵². These various signaling effects combine to produce a compensatory type of cardiac hypertrophy in RKIP-tg in response to TAC, which may contribute to the positive inotropic phenotype of RKIP-tg and includes attenuation of apoptosis and fibrosis, preservation of cardiac function, a pronounced increase of capillary density and the absence of SERCA2a reduction (Supplementary FIG. 4)²⁰. Interestingly, with regard to RKIP as a potential protective feedback mechanism in heart failure, experiments in NRCM further showed that a certain threshold concentration for RKIP is needed to sensitize βAR-signaling (Supplementary FIG. 12). This may explain the absence of an obvious phenotype of RKIP^{-/-} mice in the absence of stress stimuli, but also the detrimental phenotype of RKIP^{-/-} mice in response to chronic pressure overload.

For mechanistic reasons, it is not surprising that several features of RKIP-tg mice are similar as in GRK2-KO mice. However, they seem to achieve their hypercontractile phenotype by different mechanisms^{9,10,14}: Unlike GRK2-KO mice, RKIP-tg mice display significantly increased contractility and relaxation under basal conditions; phospholamban phosphorylation in RKIP-tg is increased; maximum L-type Ca²⁺ currents and NCX function are not affected by RKIP. These differences may result from a specific mode of GRK inhibition by RKIP that preferentially impairs phosphorylation of receptors, while phosphorylation of cytosolic targets by GRKs was less affected. A further difference in these models may involve residual unphosphorylated RKIP that might cause a modest ERK1/2 inhibition, since—unlike expected during βAR activation—phospho-ERK1/2 levels were rather unchanged compared to wild-type mice. In fact, very high overexpression of RKIP or overexpression of RKIP^{S153A} in the heart¹², can lead to ERK1/2 inhibition, and subsequently to pro-apoptotic effects⁵³ (Supplementary FIG. 13).

With our findings on RKIP, we can show for the first time that there is an endogenous protein that simultaneously facilitates different β-adrenergic effects that may alleviate heart failure: (i) gain of cardiac contractile efficiency by activation and functional recovery of β_{□}AR-Gₛ signaling and (ii) protection from exaggerated β_{□}AR downstream signaling including protection from apoptosis and pro-arrhythmic adverse effects by β₂AR-Gᵢ-mediated signaling. Our data strongly substantiate the hypothesis that a successful positive inotropic strategy should either circumvent RyR2 sensitization (similarly as e.g. PLN-KO, SERCA2a overexpression or βARKct) or even better protect from RyR2 sensitization (GRK2-KO, RKIP) as trigger for myocardial adverse effects (FIG. 6)^{13,19-32}.

In summary, our study characterizes RKIP overexpression/activation as a potential therapeutic strategy based on genetically altered mice but also on a gene therapy approach in mice. Further studies will have to show whether this strategy can be successfully translated to human heart failure, either via compounds that may change RKIP levels or activity or via gene therapy. Even though, unlike in mice, the β₂AR mediates positive inotropic effects in humans, some reports support a protective role of β₂AR also in human heart failure: the hypofunctional β₂AR Ile164 isoform was reported to be associated with a reduced prognosis of heart failure patients⁵⁴; and β₂AR-Gᵢ activation was found as a favourable component in treating Takotsubo cardiomyopathy⁵⁵⁻⁵⁷.

Thus, our data suggest the upregulation of RKIP in failing hearts holds promise as a new positive inotropic strategy in heart failure.

### Methods

### Antibodies Used for Immunodetection of Proteins

We used the following antibodies for immunoblotting or immunohistochemistry: pAkt(T308) (244F9, Cell Signaling), Akt2 (2964, Cell Signaling), β₁AR (sc-568, Santa Cruz Biotechnology), pβ₂AR(355/356) (sc-22191, Santa Cruz Biotechnology), β-tubulin (sc-9104, Santa Cruz Biotechnology), pCaMKII(T286) (MA1-047, Thermo Scientific; was used for the detection of CaMKII phosphorylation but also for CaMKII-mediated phosphorylation of LTCC at T498 as reported by Grueter et al.)⁵⁸, pCaᵥ1.2 (51928) (A010-70, Badrilla), Caᵥ1.2 (ACC-003, Alomone labs), CD31 (sc-28188, Santa Cruz Biotechnology), pERK1/2(T202/Y204) (9101, Cell Signaling), ERK1/2 (9102, Cell Signaling), pDARPP32 (T34, for detection of pI-1[T35]⁵⁹) (D27A4, Cell Signaling), pEzrin(T567)/Radixin(T564)/Moesin(T558) (3141, Cell Signaling), Gβ (sc-378, Santa Cruz Biotechnology), NCX (π11-13, Swant), p(Ser)PKC substrate antibody (2261, Cell Signaling), PKCα, (sc-208, Santa Cruz), PKCε (sc-1681, Santa Cruz), pPLN(S16) (A010-12, Badrilla), pPLN(T17) (A010-10, Badrilla), PLN (A010-14, Badrilla), PP1 (07-1218, Millipore), PP2A (05-421, Milipore), pSer/Thr (612548, BD Transduction Laboratories), pRKIP (hSer153) (sc-32623, Santa Cruz Biotechnology), pRKIP (rSer153) (sc-32622, Santa Cruz Biotechnology), RKIP (sc5422, Santa Cruz Biotechnology), RKIP antibodies raised against recombinant GST-RKIP as antigen¹⁵, pRyR2(S2808) (A010-30, Badrilla), pRyR2(S2814) (A010-31, Badrilla), RyR2 (34C, Thermo Scientific), pTnI (S23/S24) (4004, Cell Signaling) and TnI (4002, Cell Signaling).

### Generation of Adeno-Associated Virus (AAV) Vectors

The open reading frame of myc-tagged RKIP (Pebp1)¹⁷ was amplified via PCR with primers carrying a BamHI digestion site and a Kozak-sequence at the 5′ and a BsrGI site at the 3′ end. The product subcloned into the self-complementary AAV vector genome plasmid pdsCMV-MLC0.26-EGFP resulting in pdsCMV-MLC0.26-RKIP. The plasmid AAV9 vectors for expression of RKIP or enhanced GFP (control) were generated by cotransfection of pdsCMV-MLC0.26-RKIP or pdsCMV-MLC0.26-EGFP together with pDP9rs, a derivative from pDP2rs with the AAV9 cap gene from p5E18-VD2-9 using polyethylenimine. Subsequently, vectors were harvested after 48 h, purified by iodixanol step gradient centrifugation, and quantified using real-time PCR as reported before²¹.

### Preparation and Culture Conditions of Neonatal Rat Cardiomyocytes (NRCM) and Adult Mouse Cardiomyocytes

We isolated NRCM from hearts of 1-2 day-old Sprague Dawley rats as described previously^{60,61} and transduced the cells with the indicated adenoviruses encoding for Flag-RKIP, β-galactosidase (LacZ) or green fluorescent protein (GFP). NRCM were cultured in Minimum Essential Medium (MEM; PAN Biotech) containing 5% (v/v) fetal calf serum (FCS), 100 U/mL penicillin, 100 μg/mL streptomycin, 2 mM L-glutamine, 350 mg/L NaHCO₃, 30 mg/L 5-bromo-2-deoxyuridine (BrdU), and 2 mg/L vitamin B12 at 37° C. and 1% CO₂. FCS was reduced to 1% (v/v) after 24 hours followed by adenoviral transduction 48 hours after preparation. Adult cardiomyocytes were isolated by retrograde perfusion of mouse hearts and enzymatic dissociation of cells as described^{60,61}.

### Detection of RKIP Activation (Ser153-Phosphorylation)

We homogenized left ventricular tissue or cells in lysis buffer (1% [v/v] Triton-X-100, 5 mM EDTA, 300 mM NaCl, 50 mM Tris [pH7.4], supplemented with protease and phosphatase inhibitors). We removed cell debris by centrifugation (10 min, 1000×g, 4° C.) and used lysates for Western blot analysis: We determined protein concentrations and used equal amounts of protein for immunoblot analysis. We separated proteins by SDS-PAGE and transferred them to PVDF membranes. We blocked membranes for 1 h with 5% (w/v) fat-free milk dissolved in 10 mM Tris (pH7.6), 100 mM NaCl and 0.1% (v/v) Tween-20, incubated membranes with primary antibodies overnight at 4° C., thereafter with secondary antibodies conjugated with horseradish peroxidase (HRP) and used the Pierce™ ECL Plus Western blotting kit for detection. To average our immunoblot experiments and to present semi-quantitative results, we quantified immunoblot signals using ImageJ.

For the detection of RKIP phosphorylation (“activation as GRK2 inhibitor”), we used antibodies directed againt Ser153-phosphorylation of RKIP (pRKIP[hSer153], sc-32623, Santa Cruz Biotechnology; pRKIP [rSer153], sc-32622, Santa Cruz Biotechnology). For RKIP expression levels we used antibodies directed against RKIP (RKIP, sc5422, Santa Cruz Biotechnology; RKIP antibodies raised against recombinant GST-RKIP as antigen (Lorenz et al., 2003).

The activation of RKIP as GRK2 inhibitor is also described in_Lorenz et al., Nature 426: 574-579 (2003) and Deiss et al., J Biol Chem 287: 23407-23417 (2012).

### Analysis of PKA Activity

We stimulated NRCM with 10 nM isoproterenol for 10 min at 37° C. in the presence of 100 μM 3-isobutyl-1-methylxanthine (IBMX). We then lysed cells in ice cold buffer containing 20 mM HEPES, 2 mM EDTA, protease inhibitors (20 μg/mL soybean trypsin inhibitor, 0.4 mM benzamidine, 1 mM PMSF) and phosphotase inhibitors (50 mM NaF, 5 mM Na₄P₂O₇, 0.1 mM Na₃VO₄, 0.002% (w/v) NaN₃, and 100 μM IBMX). After sonification, we removed nuclear fraction and cell debris by centrifugation (25,200×g; 10 min, 4° C.). Adult cardiomyocytes were stimulated with 10 μM isoproterenol for 10 min at 30° C. in MEM containing 0.1% (m/v) BSA, 100 U/mL penicillin, 100 μg/mL streptomycin, 2 mM L-glutamine, 20 mM 2,3-butanedione monoxime (BDM), 1×ITS-supplement and 100 μM IBMX. We then homogenized the cells by sonification in 25 mM Tris-HCl (pH 7.4), 0.5 mM EDTA, 0.5 mM EGTA, 1 μg/ml leupeptin, 1 μg/ml aprotinin, 1 mM PMSF and 100 μM IBMX and removed the nuclear fraction and cell debris by centrifugation (25,200×g; 10 min, 4° C.).

We determined kinase activity of PKA by in vitro phosphorylation of C-terminal hexahistidine-tagged wild-type phosducin (phosducin-His₆). We assessed phosphorylation in a total volume of 50 μl buffer (20 mM HEPES [pH 7.2], 2 mM EDTA, 10 mM MgCl₂) containing 5 μg phosducin/sample, 50 μM [γ-³²P]ATP and 25 μg (neonatal) or 30 μg (adult) cardiomyocyte lysate for 30 min at 30° C. A phosducin mutant that lacks the PKA phosphorylation site (phosducin^{S73A}-His₆) was used as control. We separated phosphorylated phosducin by SDS-PAGE and quantified its phosphorylation by Phospholmager analysis.

cAMP-RIA

We stimulated adenovirally transduced NRCM in HEPES buffer (137 mM NaCl, 5 mM KCl, 1 mM MgCl₂, 1 mM CaCl₂, 15 mM HEPES/NaOH [pH7.3], 100μM IBMX) with the indicated concentrations of isoproterenol for 20 min at 37° C. We then lysed the cells and precipitated the proteins in 2% (v/v) HClO₄ (20 min on ice). We neutralized the suspension with 1M KOH and removed precipitate by centrifugation (10 min; 25,200×g, 4° C.). Supernatants were then used for cAMP measurements using a cAMP radioimmuno-assay kit (Beckmann&Coulter) according to the manufacturer's recommendations.

### Human Heart Samples

We obtained samples of human failing hearts from individuals undergoing heart transplantation due to end-stage heart failure (NYHA IV). Samples from seven non-failing donor hearts that could not be transplanted for technical reasons served as controls. Donor histories and echocardiography showed no signs of pathological heart function.

In accordance with the declaration of Helsinki, written informed consent had been obtained of all participants or the family of prospective heart donors before cardioectomy. All experiments were performed with the approval of the ethical committee of the Wuerzburg medical faculty or BIOSS Centre for Biological Signaling Studies, University of Freiburg.

Mice and Rats We generated transgenic mice overexpressing RKIP (RKIP-tg; Pebp1) and RKIP^{S153A} under the control of the mouse α-myosin heavy chain (α-Mhc) promoter by pronucleus injection of fertilized oocytes derived from FVB/N mice as described previously⁶⁰. For further analyses, we backcrossed RKIP and RKIP^{S153A} transgenic mice in C57BL/6J background for more than 10 generations. Isogenic, age- and gender-matched littermates were used as controls. To generate RKIP transgenic mice with homozygous deletion of β₁- or β₂AR (RKIP-tg/β₁KO or RKIP-tg/β₂KO), RKIP mice were crossed with β₁- and β₂AR knockout mice that were generated in Dr. Brian Kobilka's laboratory as described²³′⁴⁸. We bred RKIP-tg/β₁KO and RKIP-tg/β₂KO mice in mixed C57BL/6J/FVB/N backgrounds and compared them to littermate β₁KO or β₂KO control mice, respectively. RKIP-transgenic mice were born at normal Mendelian ratios in wild-type, β₁KO and β₂KO backgrounds. Of note, we avoided direct comparisons between different mouse backgrounds. This is particularly relevant for quantitative comparisons across Wt and RKIP-tg mice in the FVB/N wild-type background, in the C57BL/6J/FVB/N β₁KO background and the C57BL/6J/FVB/N β₂KO background. We only compared data of littennates.

We obtained RKIP heterozygous knockout (RKIP^{+/-}) mice from the Mutant Mouse Regional Resource Center (MMRRC, a NCRR-NIH funded strain repository at the University of California, Davis; identification number 030379-UCD). In these mice, a gene-trap vector encoding the En2 splice acceptor site fused to β-galactosidase/neo fusion gene (β-geo) was inserted between exon 3 and 4 of the mouse RKIP locus. We verified homozygous deletion of RKIP using primer sequences matching the sequence around the insertion site of the trapping vector (forward primer: 5′ GCTTTCCAGGCCTCTCAGTGTTCATCAG 3′; reverse primer: 5′ CCCGCCCATCCTGCCCATAGG 3′). We maintained homozygous RKIP^{-/-} mice on a C57BL/6J background and used age- and gender-matched C57BL/6J mice as controls. Care of the animals was taken in accordance with the Committee on Animal Research of the regional government (Regierung von Unterfranken, Germany) that reviewed and approved all experimental protocols (Az. 54-2531.01-62/06, Az. 55.2-2531.01-46/09, -20/10, -52/10, -38/11, -60/13, -41/14 and -42/14) according to the corresponding national legislation.

### Left Ventricular Catheterization

For left ventricular pressure measurements we inserted a 1.4F pressure catheter (Millar Instruments) into the right carotid artery and advanced it to the left ventricular chamber. During measurement we injected esmolol (12 μg/min for 1 min) or dobutamine (in increasing doses: 75, 150, 375, 750 and 1500 ng/min) into the V. jugularis. We analyzed data using the Chart software (Chart5.4, AD Instruments) as previously described⁶⁰. We excluded mice with heart rates below 450 bpm (or below 430 bpm in the case of β₁KO or RKIP-tg/β₁KO mice).

### Transverse Aortic Constriction

At the age of 8 weeks, we subjected Wt, RKIP-tg, RKIP^{-/-}, β₁KO, RKIP-tg/β₁KO, β₂KO or RKIP-tg/β₂KO mice to transverse aortic constriction (TAC) using a 27-gauge needle to induce chronic left ventricular (LV) pressure overload as described previously⁶⁰. Before TAC and 3 weeks (C57BL/6J background) or 6 weeks (C57BL/6J/FVB/N background) after TAC, echocardiography was performed and mice were sacrificed. Experiments in FIG. 3 were performed with RKIP-tg mice in the C57BL/6J background for comparability with RKIP^{-/-} mice. The hypercontractile, anti-apoptotic and anti-fibrotic phenotype of RKIP-tg mice in response to TAC has also been observed in the FVB/N background; however, FVB/N wild-type (as well as RKIP-tg in FVB/N background) mice tolerate chronic pressure overload-induced by TAC without functional cardiac deterioration and are, thus, FVB/N mice not applicable as a model for heart failure, which has also been observed by others⁶².

For AAV experiments, Wt or RKIP^{-/-} mice with C57BL/6J background were used as indicated. Mice were randomly assigned to the treatment groups. AAV9 vectors were intravenously injected into the tail vein as a 150 μL bolus (1×10¹² total virus particles) during TAC surgery. After 4 weeks, echocardiography was performed and mice were sacrificed. Hearts were weighed and tissue was used for histological and biochemical analyses. We excluded banded mice with an aortic pressure gradient below 60 mmHg.

### Osmotic Minipumps

Osmotic minipumps (Alzet, model 1004) were implanted subcutaneously in male 8-week old Wt and RKIP-tg mice (C57/BL/6J background). The implanted pumps continuously released isoproterenol for 4 weeks (30 mg/kg body weight/day).

### Echocardiography

We performed transthoracic echocardiograms in a blinded manner using the Vevo700 or Vevo2100 high-resolution imaging systems (VisualSonics) and a 30-MHz probe. We obtained values for end-diastolic septal and posterior wall thicknesses as well as end-diastolic and -systolic internal diameters from 2D M-mode images in the short axis view at the proximal level of the papillary muscles. We obtained peak blood flow velocities at the site of TAC [Vₘₐₓ (millimetres per second)] from pulsed-waved Doppler measurements. We calculated fractional shortening (FS), ejection fraction (EF), and aortic pressure gradients (mmHg) by VisualSonics Cardiac Measurements software. The shown data represent averages of at least six cardiac cycles per animal. We excluded mice with heart rates below 450 bpm (or below 430 bpm in the case of β₁KO or RKIP-tg/(β₁KO mice).

### Implantation of Radiotelemetry Transmitters

We implanted the ETA-F10 transmitter (Data Science International, DSI) into the peritoneal cavity and placed the negative and positive electrodes subcutaneously in lead II position. After ten days of recovery, we started ECG-measurements. For induction of arrhythmia, we administered 50 μg of isoproterenol intraperitoneally (1.5 mg/kg body weight; i.p.). We recorded ECG traces 24 hours before and isoproterenol administration using LabChart software (Chart 5.4, AD Instruments). We counted the number of ectopic beats per hour under basal conditions and after isoproterenol administration during a time period of two hours.

### Histological, Morphometric and TUNEL Analyses

After fixation of hearts in 4% (m/v) paraformaldehyde, hearts were embedded in paraffin. Sections (2 μm) were stained with haematoxylin and eosin or with Sirius Red as described previously⁶⁰. For quantification of fibrosis, we stained left ventricular sections with Sirius Red and subsequently analyzed four representative sections per animal by semi-automated image analyses in blinded manner. For immunostaining, we used Vectastain Elite ABC Reagent with biotinylated goat-anti-rabbit IgG in combination with diaminobenzidine (DAB) and glucose oxidase (Sigma) similarly as described previously^{60,61}. We counterstained nuclei with haematoxylin.

Detection of apoptotic cells was performed as previously described⁵³. After dewaxing and rehydration, we permeabilized tissue sections with proteinase K and subsequent RNase treatment. For TUNEL staining, we used an in situ cell detection kit (Roche) according to the manufacturer's protocol. Pre-treatment with DNase-I served as positive control and TUNEL reaction mixture lacking terminal transferase (TdT) as negative control. Cell nuclei and membranes were counterstained with Hoechst33258 (Sigma) and wheat germ agglutinin Samples were analyzed by fluorescence microscopy (Leica; DM 4000B). Variances between experiments (TUNEL analyses and quantification of Sirius Red staining) are due to independent batches of staining and only samples that were stained and evaluated in parallel and analyzed by the same person were directly compared.

### Analysis of Capillary-Myocyte Density

We identified endothelial cells by immunohistochemical staining of CD31 followed by a biotinylated horse anti-rabbit IgG and horse radish peroxidase (Vectastain Biozol). We counted in a blinded manner capillaries and cardiomyocytes in a total of 12 randomized visual fields (image magnification 400×) of left ventricular myocardium at which a cross section of capillaries and cardiomyocytes was clearly visible. We determined the number of capillaries per cardiomyocytes.

Treatment of Mice with Isoproterenol, ICI 118,551 and Pertussis Toxin

We intravenously infused isoproterenol (1.1 μg/min for 2 min) with or without ICI 118,551 (0.14 μg/min for 2 min) via the V. jugularis as indicated. We pretreated mice with pertussis toxin (PTX; 300 μg/kg body weight; i.p) for 24 h as indicated. Mice were sacrificed and hearts were used for Western blot analyses. Efficiency of Gαᵢ inhibition by PTX was evaluated by in vitro PTX-catalyzed ADP ribosylation in the presence of [³²P]-nicotinamide adenine dinucleotide ([³²P]NAD). For this analysis, we minced ventricular tissue of untreated control mice and mice pretreated with PTX in a buffer containing 50 mM Tris (pH7.6), 5 mM MgCl₂, 5 mM EDTA, 1 mM EGTA and 1 μg/mL aprotinin. After filtration through a 200 μm-mesh, we determined the protein content of the tissue homogenate and then incubated 30 μg of protein with 0.1 μM [³²P]NAD (800 Ci/mmol) in a buffer containing 20 mM Tris-HCl (pH7.6), 15 mM thymidine, 1 mM EDTA, 1 mM ATP, 0.1 mM GDP and 1 μg of PTX (preactivated) for 1 h at 30° C. The reaction was stopped by addition of Laemmli buffer. Proteins were separated on SDS-PAGE and incorporated [³²P] was assessed by Phospholmager analysis. ADP-ribosylation assays with tissue homogenates of PTX-treated mice resulted in a 66±8% reduction of Gαᵢ ADP-ribosylation compared to homogenates of untreated mice.

### Lysate Preparation, Immunoblot Analysis and Immunoprecipitation Experiments

We homogenized left ventricular tissue in lysis buffer (1% [v/v] Triton-X-100, 5 mM EDTA, 300 mM NaCl, 50 mM Tris [pH7.4], supplemented with protease and phosphatase inhibitors). We removed cell debris by centrifugation (10 min, 1000×g, 4° C.) and used lysates for Western blot analysis or immunoprecipitation. We determined protein concentrations and used equal amounts of protein for immunoprecipitation and immunoblot analysis. For immunoprecipitation, we preincubated protein-A sepharose with β₁AR specific antibodies overnight under continuous rotation at 4° C. (8 μl protein-A sepharose and 2 μg of β₁AR antibodies per sample). We then incubated the indicated lysates (2.4 mg/sample) with precoupled protein-A sepharose (2 h, 4° C.). After incubation, we washed beads 5 times with lysis buffer (1 ml; 20,000×g; 1 min, 4° C.). We separated proteins by SDS-PAGE and transferred them to PVDF membranes. We blocked membranes for 1 h with 5% (w/v) fat-free milk dissolved in 10 mM Tris (pH7.6), 100 mM NaCl and 0.1% (v/v) Tween-20, incubated membranes with primary antibodies overnight at 4° C., thereafter with secondary antibodies conjugated with horseradish peroxidase (HRP) and used the Pierce™ ECL Plus Western blotting kit for detection. To average our immunoblot experiments and to present semi-quantitative results, we quantified immunoblot signals using ImageJ or Photoshop.

### 2D-SDS Gel Electrophoresis

For 2D-SDS gel electrophoresis we prepared lysates of left ventricular tissue of Wt and RKIP^{-/-} mice. We subjected 2 mg of total protein to methanol/chloroform precipitation to desalt samples. We then resuspended protein pellets in sample buffer (9M urea, 4% [w/v] CHAPS, 50 mM DTT, 0.2% [w/v] ampholytes, 0.001% [m/v] bromophenol blue). We loaded 600 μg of protein on ReadyStrip IPG strips (pH4.7 to pH5.9; Biorad) via active rehydration (50V, 12 h) prior to isoelectric focusing for 45000Vh (IEF; first dimension). After IEF, we denatured and alkylated proteins according to the manufacturer's recommendations (Biorad) and performed the second dimension by SDS-PAGE. We transferred proteins onto PVDF-membranes and performed immunoblot analysis for RKIP. Equal loading was verified with Coomassie staining.

### Rhodopsin Phosphorylation

Left ventricular tissue was homogenized in buffer containing 20 mM HEPES, 2 mM EDTA, supplemented with protease and phosphatase inhibitors. Cell debris was removed by centrifugation (1,000×g; 10 min, 4° C.). We assessed kinase activity of GRK in heart lysates by in vitro phosphorylation of rhodopsin in urea-treated rod outer segment membranes in a total volume of 50 μl buffer (20 mM HEPES [pH7.4], 2 mM EDTA, 10 mM MgCl₂) containing 400 nM rod outer segment membranes, 50 μM [γ-³²P] ATP and 85 μg of cardiac protein. We initiated rhodopsin activation by light and let phosphorylation proceed for 10 min at room temperature¹⁵. Phosphorylated rhodopsin was separated by SDS-PAGE and assessed by autoradiography and Phospholmager analysis.

### Tubulin Phosphorylation and Preparation of Membrane Fractions for Phospho-β₂AR Immunoblots

Left ventricular tissue was homogenized in buffer containing 20 mM HEPES, 2 mM EDTA, supplemented with protease and phosphatase inhibitors. Nuclear fraction and cell debris was removed by centrifugation (25,200×g; 10 min, 4° C.). To assess the β₂AR phosphorylation by immunoblot analysis, the membrane fraction was obtained by centrifugation (150,000×g; 20 min, 4° C.). For tubulin phosphorylation assays, supernatants were depleted from endogenous β-tubulin by rotation of the lysates with anti-β-tubulin antibodies coupled to protein-A sepharose beads (2 h, 4° C.). We assessed kinase activity of GRK in lysates by in vitro phosphorylation of β-tubulin in a total volume of 50 μl buffer (20 mM HEPES [pH7.2], 2 mM EDTA, 10 mM MgCl₂) containing 5 μg/sample β-tubulin, 50 μM [γ-³²P] ATP and 20 μg of cytosolic or lysate protein (30 min, 30° C.). Phosphorylated 0-tubulin was separated by SDS-PAGE and assessed by autoradiography and Phospholmager analysis.

### In Vitro Phosphorylation of RKIP by PKC

We incubated purified His₆-RKIP (3 μg/sample) with recombinant PKCα, PKCδ or PKCε (100 ng/sample; Enzo Life Sciences) in kinase buffer (20 mM HEPES [pH 7.4], 10 mM MgCl₂, 0.1 mM CaCl₂, 3 mM β-mercaptoethanol, 10 μM ATP, 50 μM [γ-³²P] ATP) at 30° C. The reaction was started by addition of 2-O-tetradecanoylphorbol 13-acetate (25 μM). After 30 min, the reaction was stopped with Laemmli buffer. Proteins were separated by SDS-PAGE and detected by subsequent Phospholmager analysis.

### RNA Preparation and Real-Time PCR

We isolated RNA from left ventricles using the RNeasy®-Kit (Qiagen) and reverse transcribed total RNA using Superscript II reverse transcriptase (Invitrogen). We performed quantitative real-time PCR using the C1000 Thermal Cycler CFX96 (BioRad) and analyzed the data as previously described⁶¹. For cDNA amplification of brain natriuretic peptide (BNP), atrial natriuretic factor (ANF), collagen type III alpha 1 (COL3α1) and glyceraldehyde-3-phosphate dehydrogenase (GAPDH), we used a reaction mixture containing SsoFast EvaGreen Supermix (BioRad). We used the following primers:

GAPDH forward primer
5′ TGGCAAAGTGGAGATTGTTG 3′;
GAPDH reverse primer
5′ CATTATCGGCCTTGACTGTG 3′;
BNP forward primer
5′ GGATCGGATCCGTCAGTCGTT 3′;
BNP reverse primer
5′ AGACCCAGGCAGAGTCAGAAA 3′;
ANF forward primer
5′ ACAGATCTGATGGATTTCAAGAACCTG 3′
ANF reverse primer
5′ AGTGCGGCCCCTGCTTCCTCA 3′
COL3α1 forward primer
5′ AAACAGCAAATTCACTTACAC 3′;
COL3α1 reverse primer
5′ ACCCCCAATGTCATAGG 3′.

### Measurement of Calcium Transients and Myocyte Contraction

We measured Ca²⁺-transients by epifluorescence and myocyte contraction by sarcomere shortening using a dual-excitation single emission-system and video based sarcomere length detection system (IonOptix™). We loaded adult myocytes with Fura-2/AM (1 μM; Invitrogen) for 15 min. The myocytes were then superperfused with prewarmed buffer (137 mM NaCl, 5.4 mM KCl, 1.2 mM CaCl₂, 1 mM MgCl₂, 10 mM HEPES, glucose 5.5 mM; pH7.4; 27° C.) and were field-stimulated at 0.5 Hz and 20V. We monitored Ca²⁺-transients in myocytes by alternating (240 Hz) dual excitation at 340 and 380 nm and emission collected at 480-520 nm using an IonOptix setup. Fura-2 fluorescence ratio was calculated by division of the signal obtained at 340 nm by that at 380 nm (F340/F380). We evaluated relaxation time (time to baseline 50% and 90%), amplitude of transients (peak height) and % of sarcomere shortening of 7-10 cells per mouse and 7-10 mice per genotype using the IonWizard® software. For NCX function we stimulated cells locally through a 21G syringe with caffeine (10 mM) and analyzed peak height and values of caffeine-induced intracellular Ca²⁺-transient decay.

### Measurement of Calcium Sparks

We incubated cardiomyocytes with Fluo-4/AM (10 μM) for 15 min in a loading buffer (see above) containing Pluronic F-127 (0.2 mg/ml). We superfused myocytes with normal tyrode (4 mM KCl, 140 mM NaCl, 1 mM MgCl₂, 5 mM HEPES, 1 mM glucose, 1 mM CaCl₂; 21° C.; pH7.4). Using a laser-scanning confocal microscope (LSMS, Zeiss), Fluo-4 was excited at 488 nm and emission was collected through a 505 nm long-pass filter. We recorded fluorescence images in line-scan mode with 512 pixels per 57.59 μm wide scanline. Following 0.5 Hz stimulation, Ca²⁺-spark frequency was measured at resting conditions and normalized to myocyte width and scanning interval (sparks/100 μm/s). Spark size was calculated as product of spark amplitude (F/F₀), full spark width and full spark duration (half-maximum spark size, the product of spark amplitude, full-width-half-maximum spark width and full-duration half-maximum spark duration were also calculated and showed very similar results). We calculated total diastolic sarcoplasmatic reticulum Ca²⁺-leak for sparking cells as the sum of all sparks in that cell during the scan (i.e. equivalent to the product of spark frequency and spark size). Of note, cardiomyocytes displaying spontaneous Ca²⁺-waves were not used for spark evaluation, as the waves will deplete SR calcium, meaning different SR Ca²⁺-loading conditions before and after the wave. However, the percentage of cells with calcium waves out of the total number of cells investigated was used as an additional measure for arrhythmogenesis.

### L-Type Ca²⁺ Current Measurements

Patch clamp experiments were performed using the EPC-10 amplifier and Patchmaster software (HEKA, Germany). Ca²⁺ currents (I_{Ca,L}) were recorded by voltage clamp using the ruptured-patch whole cell patch clamp technique. After rupture, equilibration of intracellular solution and cytosol was allowed for 2 min before starting recordings. Fast and slow capacitance as well as series resistance were compensated for using the built-in functions of Patchmaster. Pipettes for I_{Ca,L} experiments were pulled to resistances of 2-2.5 MΩ and filled with Na⁺- and K⁺-free intracellular solution containing 90 mM Cs-methanesulfonate, 20 mM CsCl, 10 mM HEPES, 4 mM MgATP, 0.4 mM Tris-GTP, 3 mM CaCl₂, 10 mM EGTA (pH7.2 at 21° C.). Liquid junction potential (LJP) was −15 mV as calculated with JPCalc V 2.2. Current measurements were not corrected for LJP. Myocytes were superfused with K-free external solution containing 120 mM tetraethyl ammonium chloride, 10 mM CsCl, 10 mM HEPES, 10 mM glucose, 1 mM MgCl₂, and 2 mM CaCl₂ (017.4 at 21° C.). Current-voltage relationships were established as follows: From a holding potential of −80 mV, cells were clamped to test potentials between −40 mV and +60 mV for a duration of 200 ms in 10 mV steps increasing with an interval of 1 s. Measured currents were normalized to membrane capacitance.

### Membrane Preparation and β-Adrenergic Receptor Density

We homogenized left ventricles of indicated mouse hearts in buffer (10 mM HEPES, 10 mM MgCl₂, 2 mM EDTA, 400 mM KCl and protease inhibitors; pH7.5). We removed intact cells and debris by centrifugation (1,700×g; 10 min, 4° C.) and next centrifuged the supernatants (50,000×g; 30 min, 4° C.) to separate cytosolic from membrane fraction. We resuspended the pellet in resuspension buffer (32 mM HEPES, 80 mM NaCl, 4 mM EGTA, 4 mM EDTA, 12.5 mM MgCl₂ and protease inhibitors; pH7.5) and determined receptor density by incubating 50 μg of plasma membranes with saturating concentrations (300 pM) of [¹²⁵I]cyanopindolol in the presence or absence of 100 μM alprenolol (non-specific binding) for 90 min at 37° C. Probes were passed through glass fibre filters (Millipore) wetted in 0.3% (v/v) polyethylenimine, followed by three washing steps with ice-cold 50 mM Tris-HCl (pH7.6). Afterwards membrane bound radioligand was measured in a γ-counter for 1 min.

### Statistical Analysis

Histological and echocardiographic analyses were performed in a blinded manner (genotype and treatment blinded). For adequate power a sample size of at least n=4 was chosen. Our results represent the mean±standard error (mean±SEM). We used t-test analysis for two-group comparisons and one-way analysis of variance analyses (ANOVA; ordinary one-way ANOVA) if more than two groups were compared. We used Bonferroni test as post-hoc test if not stated otherwise. The occurrence of Ca²⁴-waves was tested using Fisher's exact test. For the analyses, we used GraphPad software (San Diego, USA). p<0.05 was regarded as significant.

### REFERENCES

1. McMurray, J. J. V. et al. ESC Guidelines for the diagnosis and treatment of acute and chronic heart failure 2012: The Task Force for the Diagnosis and Treatment of Acute and Chronic Heart Failure 2012 of the European Society of Cardiology. Developed in collaboration with the Heart Failure Association (HFA) of the ESC. Eur Heart J 14, 803-869 (2012).
2. Tacon, C. L., McCaffrey, J. & Delaney, A. Dobutamine for patients with severe heart failure: a systematic review and meta-analysis of randomised controlled trials. Intensive Care Med 38, 359-367 (2011).
3. Engelhardt, S., Hein, L., Wiesmann, F. & Lohse, M. J. Progressive hypertrophy and heart failure in 13₁-adrenergic receptor transgenic mice. Proc Natl Acad Sci USA 96, 7059-7064 (1999).
4. Communal, C., Singh, K., Sawyer, D. B. & Colucci, W. S. Opposing Effects of β₁- and β₂-Adrenergic Receptors on Cardiac Myocyte Apoptosis: Role of a Pertussis Toxin-Sensitive G Protein. Circulation 100, 2210-2212 (1999).
5. Liggett, S. B. et al. Early and Delayed Consequences of β₂-Adrenergic Receptor Overexpression in Mouse Hearts: Critical Role for Expression Level. Circulation 101, 1707-1714 (2000).
6. Roclunan, H. A., Koch, W. J. & Lefkowitz, R. J. Seven-transmembrane-spanning receptors and heart function. Nature 415, 206-212 (2002).
7. Xiao, R. P. β-Adrenergic Signaling in the Heart: Dual Coupling of the β₂-Adrenergic Receptor to Gs and Gi Proteins. Science's STKE 2001, 15re-15 (2001).
8. Ahmet, I. et al. Cardioprotective and Survival Benefits of Long-Term Combined Therapy with β₂ Adrenoreceptor (AR) Agonist and β₁ AR Blocker in Dilated Cardiomyopathy Postmyocardial Infarction. J Pharmacol Exp Ther 325, 491-499 (2008).
9. Raake, P. W. et al. G Protein-Coupled Receptor Kinase 2 Ablation in Cardiac Myocytes Before or After Myocardial Infarction Prevents Heart Failure. Circ Res 103, 413-422 (2008).
10. Raake P. W., et al. Cardiac G-Protein-Coupled Receptor Kinase 2 Ablation Induces a Novel Ca²⁺ Handling Phenotype Resistant to Adverse Alterations and Remodeling After Myocardial Infarction. Circulation 125, 2108-2118 (2012).
11. Völkers, M. et al. The Inotropic Peptide βARKct Improves AR Responsiveness in Normal and Failing Cardiomyocytes Through Gβγ-Mediated L-Type Calcium Current Disinhibition. Circ Res 108, 27-39 (2011).
12. Fu, X., Koller, S., Abd Alla, J. & Quitterer, U. Inhibition of G-protein-coupled Receptor Kinase 2 (GRK2) Triggers the Growth-promoting Mitogen-activated Protein Kinase (MAPK) Pathway. J Biol Chem 288, 7738-7755 (2013).
13. Kairouz, V., Lipskaia, L., Hajjar, R. J. & Chemaly, E. R. Molecular targets in heart failure gene therapy: current controversies and translational perspectives. Ann N Y Acad Sci 1254, 42-50 (2012).
14. Matkovich, S. J. et al. Cardiac-Specific Ablation of G-Protein Receptor Kinase 2 Redefines Its Roles in Heart Development and 13-Adrenergic Signaling. Circ Res 99, 996-1003 (2006).
15. Lorenz, K., Lohse, M. J. & Quitterer, U. Protein kinase C switches the Raf kinase inhibitor from Raf-1 to GRK-2. Nature 426, 574-579 (2003).
16. Yeung, K. et al. Suppression of Raf-1 kinase activity and MAP kinase signalling by RKIP. Nature 401, 173-177 (1999).
17. Deiss, K., Kisker, C., Lohse, M. J. & Lorenz, K. Raf Kinase Inhibitor Protein (RKIP) Dimer Formation Controls Its Target Switch from Raft to G Protein-coupled Receptor Kinase (GRK) 2. J Biol Chem 287, 23407-23417 (2012).
18. Palczewski, K., Buczylko, J., Lebioda, L., Crabb, J. W. & Polans, A. S. Identification of the N-terminal Region in Rhodopsin Kinase Involved in its Interaction with Rhodopsin. J Biol Chem 268, 6004-6013 (1993).
19. Lehnart, S. E., Maier, L. S. & Hasenfuss, G. Abnormalities of calcium metabolism and myocardial contractility depression in the failing heart. Heart Fail Rev 14, 213-224 (2009).
20. Maillet, M., van Berlo, J. H. & Molkentin, J. D. Molecular basis of physiological heart growth: fundamental concepts and new players. Nat Rev Mol Cell Bio 14, 38-48 (2013).
21. Pleger, S. T. et al. Cardiac AAV9-S100A1 Gene Therapy Rescues Post-Ischemic Heart Failure in a Preclinical Large Animal Model. Sci Transl Med 3, 92ra64 (2011).
22. Rohrer, D. K., Schauble, E. H., Desai, K. H., Kobilka, B. K. & Bernstein, D. Alterations in dynamic heart rate control in the β₁-adrenergic receptor knockout mouse. Am J Physiol 274, H1184-93 (1998).
23. Chruscinski, A. J. et al. Targeted disruption of the β2 Adrenergic Receptor Gene. J Biol Chem 274, 16694-16700 (1999).
24. Ling, H. et al. Requirement for Ca²⁺/calmodulin-dependent kinase II in the transition from pressure overload-induced cardiac hypertrophy to heart failure in mice. J Clin Invest 119, 1230-1240 (2009).
25. Shiferaw Y., Aistrup G. L., Wasserstrom J. A. Intracellular Ca²⁺ waves, afterdepolarizations, and triggered arrhythmias. Cardiovasc Res 95, 265-268 (2012).
26. Guo, T., Zhang T., Mestril R. & Bers D. M. Ca²⁺/Calmodulin-Dependent Protein Kinase II Phosphorylation of Ryanodine Receptor Does Affect Calcium Sparks in Mouse Ventricular Myocytes. Circ Res 99, 398-406 (2006).
27. Bers, D. M. Ryanodine Receptor S2808 Phosphorylation in Heart Failure: Smoking Gun or Red Herring. Circ Res 110, 796-799 (2012).
28. Eschenhagen, T. Is ryanodine receptor phosphorylation key to the fight or flight response and heart failure? J Clin Invest 120, 4197-4203 (2010).
29. Respress, J. L. et al. Role of RyR2 Phosphorylation at S2814 During Heart Failure Progression. Circ Res 110, 1474-1483 (2012).
30. Dobrev, D. & Wehrens, X. H. T. Role of RyR2 Phosphorylation in Heart Failure and Arrhythmias: Controversies Around Ryanodine Receptor Phosphorylation in Cardiac Disease. Circ Res 114, 1311-1319 (2014).
31. Shan, J. et al. Phosphorylation of the ryanodine receptor mediates the cardiac fight or flight response in mice. J Clin Invest 120, 4388-4398 (2010).
32. Anderson, M. E., Brown, J. H. & Bers, D. M. CaMKII in myocardial hypertrophy and heart failure. J Mol Cell Cardiol 51, 468-473 (2011).
33. Orchard, C. & Brette, F. t-tubules and sarcoplasmic reticulum function in cardiac ventricular myocytes. Cardiovasc. Res. 77, 237-244 (2007).
34. Nikolaev, V. O. et al. β₂-Adrenergic Receptor Redistribution in Heart Failure Changes cAMP Compartmentation. Science 327, 1653-1657 (2010).
35. Kuschel, M. et al. Gᵢ Protein-mediated Functional Compartmentalization of Cardiac β₂-Adrenergic Signaling. J Biol Chem 274, 22048-22052 (1999).
36. Xiao, R. P. et al. Coupling of β₂-adrenoceptor to Gᵢ proteins and its Physiological Relevance in Murine Cardiac Myocytes. Circ Res 84, 43-52 (1999).
37. Daaka, Y., Luttrell, L. M. & Lefkowitz, R. J. Switching of the coupling of the β₂-adrenergic receptor to different G proteins by protein kinase A. Nature 390, 88-91 (1997).
38. Talan, M. I., Ahmet, I., Xiao, R.-P. & Lakatta, E. G. β₂AR Agonists in Treatment of Chronic Heart Failure: Long Path to Translation. J Mol Cell Cardiol 51, 529-533 (2011).
39. Chesley, A. et al. The β₂-Adrenergic Receptor Delivers an Antiapoptotic Signal to Cardiac Myocytes Through Gᵢ-Dependent Coupling to Phosphatidylinositol 3′-Kinase. Circ Res 87, 1172-1179 (2000).
40. Slack, J. The Enhanced Contractility of the Phospholamban-deficient Mouse Heart Persists with Aging. J Mol Cell Cardiol 33, 1031-1040 (2001).
41. Kranias, E. G. & Hajjar, R. J. Modulation of Cardiac Contractility by the Phopholamban/SERCA2a Regulatome. Circ Res 110, 1646-1660 (2012).
42. Kho, C. et al. SUMO1-dependent modulation of SERCA2a in heart failure. Nature 477, 601-605 (2012).
43. Greenberg, B. et al. Design of a Phase 2b Trial of Intracoronary Administration of AAV1/SERCA2a in Patients With Advanced Heart Failure. JACC: Heart Failure 2, 84-92 (2014).
44. Antos, C. L. et al. Dilated Cardiomyopathy and Sudden Death Resulting From Constitutive Activation of Protein Kinase A. Circ Res 89, 997-1004 (2001).
45. El-Armouche, A. et al. Phosphatase inhibitor-1-deficient mice are protected from catecholamine-induced arrhythmias and myocardial hypertrophy. Cardiovasc Res 80, 396-406 (2008).
46. Jurevicius, J. & Fischmeister, R. cAMP compartmentation is responsible for a local activation of cardiac Ca²⁺ channels by β-adrenergic agonists. Proc Natl Acad Sci USA 93, 295-299 (1996).
47. Koval, O. M. et al. Caᵥ1.2 β-subunit coordinates CaMKII-triggered cardiomyocyte death and afterdepolarizations. Proc Natl Acad Sci USA 107, 4996-5000 (2010).
48. Rohrer, D. K. et al. Targeted disruption of the mouse β₁-adrenergic receptor gene: developmental and cardiovascular effects. Proc Natl Acad Sci USA 93, 7375-7380 (1996).
49. Rohrer, D. K., Chruscinski, A., Schauble, E. H., Bernstein, D. & Kobilka, B. K. Cardiovascular and Metabolic Alterations in Mice Lacking Both β1- and β2-Adrenergic Receptors. J Biol Chem 274, 16701-16708 (1999).
50. Braz, J. C. et al. PKC-α regulates cardiac contractility and propensity toward heart failure. Nat Med 10, 248-254 (2004).
51. Hambleton, M. et al. Pharmacological- and Gene Therapy-Based Inhibition of Protein Kinase Cα/β Enhances Cardiac Contractility and Attenuates Heart Failure. Circulation 114, 574-582 (2006).
52. Dorn, G. W. et al. Sustained in vivo cardiac protection by a rationally designed peptide that causes 6 protein kinase C translocation. Proc Natl Acad Sci USA 96, 12798-12803 (1999).
53. Ruppert, C. et al. Interference with ERK(Thr188) phosphorylation impairs pathological but not physiological cardiac hypertrophy. Proc Natl Acad Sci USA 110, 7440-7445 (2013).
54. Liggett, S. B. et al. The Ile164 β₂-Adrenergic Receptor Polymorphism Adversely Affects the Outcome of Congestive Heart Failure. J Clin Invest 102, 1534-1539 (1998).
55. Siedlecka, U. et al. Effects of clenbuterol on contractility and Ca²⁺ homeostasis of isolated rat ventricular myocytes. Am J Physiol Heart Circ Physiol 295, H1917-H1926 (2008).
56. Paur, H. et al. High Levels of Circulating Epinephrine Trigger Apical Cardiodepression in a β₂-Adrenergic Receptor/Gᵢ-Dependent Manner: A New Model of Takotsubo Cardiomyopathy. Circulation 126, 697-706 (2012).
57. Shao, Y. et al. Novel rat model reveals important roles of β-adrenoreceptors in stress-induced cardiomyopathy. Int J Cardiol 168, 1943-1950 (2013).
58. Grueter, C. E., Abiria, S. A., Wu, Y., Anderson, M. E. & Colbran, R. J. Differential Regulated Interactions of Calcium/Calmodulin-Dependent Protein Kinase II with Isoforms of Voltage-Gated Calcium Channel β Subunits. Biochemistry 47, 1760-1767 (2008).
59. El-Armouche, A., Pamminger, T., Ditz, D., Zolk, O., Eschenhagen, T. Decreased protein and phosphorylation level of the protein phosphatase inhibitor-1 in failing human hearts. Cardiovasc Res 61, 87-93 (2004).
60. Lorenz, K., Schmitt, J. P., Schmitteckert, E. M. & Lohse, M. J. A new type of ERK1/2 autophosphorylation causes cardiac hypertrophy. Nat Med 15, 75-83 (2008).
61. Vidal, M., Wieland, T., Lohse, M. J. & Lorenz, K. β-Adrenergic receptor stimulation causes cardiac hypertrophy via a Gβγ/Erk-dependent pathway. Cardiovasc Res 96, 255-264 (2012).
62. Nakayama, H. et al. Ca²⁺- and mitochondrial-dependent cardiomyocyte necrosis as a primary mediator of heart failure. J Clin Invest 117, 2431-2444 (2007).

SUPPLEMENTARY TABLE 1

| | | RKIP-tg | RKIP^{S153A} | RKIP^{S153A} |
|---|---|---|---|---|
| | Wt | line #4 | line #10 | line #13 |
| IVS, | 0.78 ± 0.007 | 0.76 ± 0.020 | 0.74 ± 0.014 | 0.76 ± 0.036 |
| end-diastolic | | | | |
| (mm) | | | | |
| LVPW, | 0.80 ± 0.024 | 0.80 ± 0.027 | 0.72 ± 0.014 | 0.79 ± 0.017 |
| end-diastolic | | | | |
| (mm) | | | | |
| LVID, | 3.22 ± 0.052 | 3.18 ± 0.066 | 3.08 ± 0.049 | 3.26 ± 0.063 |
| end-diastolic | | | | |
| (mm) | | | | |
| FS | 36.7 ± 0.91 | 43.6 ± 1.57* | 37.7 ± 1.03 | 36.3 ± 1.81 |
| (% of LVID) | | | | |
| EF | 67.6 ± 1.19 | 75.6 ± 1.68* | 68.8 ± 1.34 | 67.0 ± 2.47 |
| (%) | | | | |
| Heart rate | 532 ± 11 | 559 ± 14 | 520 ± 11 | 533 ± 12 |
| (bpm) | | | | |
| n | 14 | 11 | 13 | 6 |

SUPPLEMENTARY TABLE 2

| | Wt | RKIP-tg | Wt | RKIP-tg |
|---|---|---|---|---|
| | Con | Con | Iso | Iso |
| IVS, end-diastolic | 0.62 ± 0.014 | 0.61 ± 0.022 | 0.74 ± 0.039* | 0.81 ± 0.026* |
| (mm) | | | | |
| LVPW, end-diastolic | 0.60 ± 0.019 | 0.59 ± 0.020 | 0.76 ± 0.036* | 0.84 ± 0.022* |
| (mm) | | | | |
| LVID, end-diastolic | 3.83 ± 0.038 | 3.65 ± 0.057 | 3.69 ± 0.172 | 3.08 ± 0.086 |
| (mm) | | | | |
| FS | 30.8 ± 1.25 | 40.6 ± 1.45^{$} | 41.6 ± 2.00^{$} | 55.9 ± 1.98*^{#} |
| (% of LVID) | | | | |
| EF | 59.0 ± 1.85 | 72.0 ± 1.74^{$} | 73.0 ± 2.52^{$} | 87.1 ± 1.64*^{#} |
| (%) | | | | |
| Heart rate | 464 ± 14 | 484 ± 12 | 654 ± 25* | 668 ± 21* |
| (bpm) | | | | |
| n | 8 | 10 | 9 | 8 |
| Interstitial fibrosis | 0.19 ± 0.029 | 0.32 ± 0.11 | 2.05 ± 0.336* | 1.05 ± 0.263*^{#} |
| (arbitrary units) | | | | |
| n | 8 | 7 | 8 | 6 |

SUPPLEMENTARY TABLE 3

| | Wt | RKIP-tg | RKIP^{S153A} |
|---|---|---|---|
| Ca²⁺-transient amplitude | 0.72 ± 0.040 | 0.88 ± 0.034*^{#} | 0.69 ± 0.030 |
| (F_{380/340}) | | | |
| 90% Ca²⁺-transient decay time | 267 ± 8 | 229 ± 5*^{#} | 266 ± 6 |
| (ms) | | | |
| 50% Ca²⁺-transient decay time | 103 ± 2 | 97 ± 1* | 102 ± 1 |
| (ms) | | | |
| Number of mice (7-10 | 12 | 12 | 7 |
| cells/mouse) | | | |
| Sarcomere shortening | 7.4 ± 0.63 | 8.5 ± 0.75^{#} | 5.8 ± 0.63 |
| (%) | | | |
| Time to 90% relaxation | 190 ± 5 | 144 ± 8*^{#} | 172 ± 6 |
| (ms) | | | |
| Time to 50% relaxation | 96 ± 3.1 | 83 ± 2.8*^{#} | 97 ± 5.0 |
| (ms) | | | |
| Number of mice (7-10 | 11 | 12 | 7 |
| cells/mouse) | | | |

SUPPLEMENTARY TABLE 4

| | Wt | RKIP-tg | RKIP^{S153A} |
|---|---|---|---|
| Basal Ca²⁺-transient | 0.28 ± 0.022 | 0.38 ± 0.033* | 0.29 ± 0.027 |
| amplitude (F_{380/340}) | | | |
| +10 mM caffeine | | | |
| Caffeine Ca²⁺-transient | 0.40 ± 0.031 | 0.50 ± 0.034* | 0.38 ± 0.031 |
| amplitude (F_{380/340}) | | | |
| τ_{Ca} | 1.9 ± 0.15 | 1.8 ± 0.097 | 1.9 ± 0.20 |
| (ms) | | | |
| Number of cells | 20 | 20 | 18 |

SUPPLEMENTARY TABLE 5

| | Wt | RKIP-tg | RKIP^{S153A} |
|---|---|---|---|
| IVS, end-diastolic | 0.91 ± 0.018 | 0.94 ± 0.019 | 0.81 ± 0.028* |
| (mm) | | | |
| LVPW, end-diastolic | 0.90 ± 0.017 | 0.94 ± 0.019 | 0.80 ± 0.017* |
| (mm) | | | |
| LVID, end-diastolic | 3.63 ± 0.111 | 3.24 ± 0.091* | 3.67 ± 0.075 |
| (mm) | | | |
| FS | 36.1 ± 1.67 | 46.2 ± 1.70* | 32.1 ± 1.22 |
| (% of LVID) | | | |
| EF | 66.7 ± 2.42 | 78.0 ± 1.65* | 60.9 ± 1.79 |
| (%) | | | |
| Heart rate | 544 ± 15 | 545 ± 10 | 534 ± 7.6 |
| (bpm) | | | |
| n | 13 | 17 | 12 |

SUPPLEMENTARY TABLE 6

| | Wt | RKIP-tg | RKIP^{-/-} | Wt | RKIP-tg | RKIP^{-/-} |
|---|---|---|---|---|---|---|
| | Con | Con | Con | TAC | TAC | TAC |
| IVS, end-diastolic | 0.64 ± 0.018 | 0.69 ± 0.034 | 0.66 ± 0.030 | 0.95 ± 0.022* | 0.99 ± 0.025* | 0.91 ± 0.035* |
| (mm) | | | | | | |
| LVPW, end-diastolic | 0.65 ± 0.018 | 0.71 ± 0.035 | 0.66 ± 0.017 | 0.90 ± 0.026* | 0.95 ± 0.034* | 0.93 ± 0.032* |
| (mm) | | | | | | |
| LVID, end-diastolic | 3.62 ± 0.055 | 3.31 ± 0.091 | 3.82 ± 0.114 | 3.91 ± 0.081* | 3.31 ± 0.091‡ | 4.63 ± 0.140*† |
| (mm) | | | | | | |
| FS | 32.6 ± 1.08 | 40.0 ± 1.14§ | 33.8 ± 1.39 | 24.6 ± 0.99* | 36.0 ± 1.81‡ | 17.4 ± 1.56*† |
| (% of LVID) | | | | | | |
| EF | 61.1 ± 1.57 | 72.2 ± 1.37§ | 63.2 ± 1.94 | 47.3 ± 1.88* | 66.4 ± 2.41‡ | 36.0 ± 2.91*† |
| (%) | | | | | | |
| Heart rate | 495 ± 12 | 520 ± 16 | 492 ± 17 | 503 ± 11 | 482 ± 18 | 510 ± 14 |
| (bpm) | | | | | | |
| Gradient | | | | 77 ± 4.2 | 73 ± 2.3 | 75 ± 2.1 |
| (mmHg) | | | | | | |
| n | 12 | 8 | 8 | 21 | 13 | 14 |
| HW/TL | 5.8 ± 0.16 | 5.5 ± 0.12 | 6.2 ± 0.15 | 9.8 ± 0.27* | 9.1 ± 0.33* | 10.3 ± 0.41* |
| (mg mm⁻¹) | | | | | | |
| n | 28 | 29 | 17 | 21 | 13 | 11 |

SUPPLEMENTARY TABLE 7

| | | Wt+ | Wt+ |
|---|---|---|---|
| | Wt | AAV9-GFP | AAV9-RKIP |
| | Con | TAC | TAC |
| IVS, end-diastolic | 0.70 ± 0.015 | 0.94 ± 0.019* | 1.01 ± 0.046* |
| (mm) | | | |
| LVPW, end-diastolic | 0.70 ± 0.017 | 0.96 ± 0.014* | 0.98 ± 0.025* |
| (mm) | | | |
| LVID, end-diastolic | 3.89 ± 0.063 | 4.24 ± 0.104*^{#} | 3.83 ± 0.092 |
| (mm) | | | |
| FS | 32.7 ± 0.69 | 21.1 ± 0.73*^{#} | 38.0 ± 2.69* |
| (% of LVID) | | | |
| EF | 61.7 ± 1.01 | 43.2 ± 1.28*^{#} | 67.9 ± 3.37 |
| (%) | | | |
| Heart rate | 512 ± 11 | 527 ± 18 | 518 ± 13 |
| (bpm) | | | |
| Gradient | | 73 ± 2.7 | 77 ± 4.4 |
| (mmHg) | | | |
| n | 22 | 11 | 11 |
| HW/TL | 7.1 ± 0.11 | 9.7* ± 0.95 | 10.2* ± 0.31 |
| (mg mm⁻¹) | | | |
| n | 14 | 12 | 12 |

SUPPLEMENTARY TABLE 8

| | | RKIP^{-/-} + | RKIP^{-/-} + |
|---|---|---|---|
| | RKIP^{-/-} | AAV9-GFP | AAV9-RKIP |
| | Con | TAC | TAC |
| IVS, end-diastolic | 0.70 ± 0.014 | 0.97 ± 0.046* | 1.0 ± 0.040* |
| (mm) | | | |
| LVPW, end-diastolic | 0.71 ± 0.013 | 0.89 ± 0.034* | 0.98 ± 0.032* |
| (mm) | | | |
| LVID, end-diastolic | 3.66 ± 0.092 | 4.61 ± 0.117*^{#} | 3.73 ± 0.106 |
| (mm) | | | |
| FS | 35.9 ± 1.44 | 15.8 ± 1.28*^{#} | 33.2 ± 2.71 |
| (% of LVID) | | | |
| EF | 65.9 ± 1.93 | 33.3 ± 2.50*^{#} | 61.8 ± 3.94 |
| (%) | | | |
| Heart rate | 560 ± 15 | 511 ± 21 | 560 ± 35 |
| (bpm) | | | |
| Gradient | | 89 ± 4.4 | 80 ± 5.7 |
| (mmHg) | | | |
| n | 19 | 9 | 10 |
| HW/TL | 7.2 ± 0.11 | 12.5 ± 0.70* | 11.4 ± 0.50* |
| (mg mm⁻¹) | | | |
| n | 13 | 10 | 11 |

SUPPLEMENTARY TABLE 9

| | | RKIP-tg/ | | RKIP-tg/ |
|---|---|---|---|---|
| | β₁KO | β₁KO | β₁KO | β₁KO |
| | Con | Con | TAC | TAC |
| IVS, end-diastolic | 0.68 ± 0.016 | 0.68 ± 0.024 | 1.08 ± 0.035* | 0.99 ± 0.023* |
| (mm) | | | | |
| LVPW, end-diastolic | 0.71 ± 0.014 | 0.70 ± 0.018 | 0.97 ± 0.020* | 0.95 ± 0.014* |
| (mm) | | | | |
| LVID, end-diastolic | 3.73 ± 0.067 | 3.47 ± 0.129 | 3.39 ± 0.132 | 3.14 ± 0.098^{‡} |
| (mm) | | | | |
| FS | 33.2 ± 0.68 | 32.3 ± 1.33 | 36.0 ± 0.85* | 39.9 ± 0.92*^{†} |
| (% of LVID) | | | | |
| EF | 62.3 ± 0.89 | 61.5 ± 1.83 | 66.5 ± 1.17* | 71.7 ± 1.13*^{†} |
| (%) | | | | |
| Heart rate | 458 ± 13 | 474 ± 15 | 482 ± 15 | 479 ± 17 |
| (bpm) | | | | |
| Gradient | | | 66 ± 2.1 | 69 ± 2.6 |
| (mmHg) | | | | |
| n | 7 | 6 | 12 | 12 |
| HW/TL | 5.8 ± 0.10 | 5.6 ± 0.09 | 8.6 ± 0.38* | 8.3 ± 0.23* |
| (mg mm⁻¹) | | | | |
| n | 27 | 28 | 12 | 12 |

SUPPLEMENTARY TABLE 10

| | | RKIP-tg/ | | RKIP-tg/ |
|---|---|---|---|---|
| | β₂KO | β₂KO | β₂KO | β₂KO |
| | Con | Con | TAC | TAC |
| IVS, end-diastolic | 0.71 ± 0.017 | 0.70 ± 0.012 | 1.05 ± 0.035* | 1.11 ± 10.024* |
| (mm) | | | | |
| LVPW, end-diastolic | 0.70 ± 0.017 | 0.71 ± 0.008 | 1.00 ± 0.034* | 1.06 ± 0.025* |
| (mm) | | | | |
| LVID, end-diastolic | 2.83 ± 0.047 | 2.92 ± 0.051 | 2.99 ± 0.074 | 2.96 ± 0.062 |
| (mm) | | | | |
| FS | 34.2 ± 1.59 | 42.4 ± 1.21^{§} | 38.4 ± 1.57 | 32.3 ± 0.90^{$#} |
| (% of LVID) | | | | |
| EF | 64.1 ± 2.15 | 74.5 ± 1.34^{§} | 69.6 ± 1.89 | 62.1 ± 1.03^{$#} |
| (%) | | | | |
| Heart rate | 514 ± 16 | 516 ± 11 | 509 ± 19 | 514 ± 16 |
| (bpm) | | | | |
| Gradient | | | 69 ± 3.3 | 68 ± 2.6 |
| (mmHg) | | | | |
| n | 13 | 14 | 13 | 16 |
| HW/TL | 5.2 ± 0.08 | 5.1 ± 0.05 | 8.0 ± 0.27* | 8.8 ± 0.35* |
| (mg mm⁻¹) | | | | |
| n | 22 | 20 | 13 | 14 |

SUPPLEMENTARY TABLE 11

| | | RKIP-tg/ | | RKIP-tg/ |
|---|---|---|---|---|
| | β₁KO | β₁KO | β₂KO | β₂KO |
| Ca²⁺-transient amplitude | 0.66 ± 0.019 | 0.69 ± 0.036 | 0.57 ± 0.020 | 0.77 ± 0.035* |
| (F_{340/380}) | | | | |
| 90% Ca²⁺-transient decay time | 395 ± 15 | 402 ± 22 | 435 ± 12 | 375 ± 17* |
| (ms) | | | | |
| 50% Ca²⁺-transient decay time | 133 ± 4 | 128 ± 5 | 141 ± 2 | 138 ± 7 |
| (ms) | | | | |
| number of mice (7-10 | 10 | 9 | 8 | 9 |
| cells/mouse) | | | | |
| Sarcomere shortening | 5.9 ± 0.35 | 5.5 ± 0.25 | 5.9 ± 0.49 | 7.9 ± 0.72* |
| (%) | | | | |
| Time to 90% relaxation | 286 ± 20 | 272 ± 31 | 298 ± 21 | 240 ± 14* |
| (ms) | | | | |
| Time to 50% relaxation | 131 ± 9 | 122 ± 13 | 127 ± 3 | 137 ± 8 |
| (ms) | | | | |
| number of mice (7-10 | 10 | 10 | 10 | 10 |
| cells/mouse) | | | | |

### EXAMPLE 2: AORTIC STENOSIS PATIENTS

### Background:

Aortic valve stenosis (AS) is the most frequent cause for valve surgeries in Europe as well as Northern America. Given its prevalence and the demographic changes, there will be a significant burden for healthcare systems in the future (Nkomo et al., 2006; see below). Understanding of this disease has changed in the last decades. In the late 1980s AS was commonly thought to be a unidirectional degenerative disease, limited solely on the valve. In the last decades a lot of research was done and now, it is widely agreed that a complex multifactorial pathophysiology underlies AS, involving both the myocardium as well as the vessels. In 1980 Carabello et al. described a set of patients with aortic valve stenosis undergoing aortic valve replacement (AVR) with poor outcome (Carabello et al., 1980; see below). Unlike the “classic” AS-patients who recovered from a decreased left ventricular (LV) ejection fraction by AVR, they described four patients who did not. Although these four patients had severe AS, the mean transvalvular pressure gradient (Pₘₑₐₙ) was than lower 30 mm Hg. This group has been in the focus of research for some years and has now been termed low gradient AS (LG/AS, Pₘₑₐₙ<40 mm Hg) (Monin et al., 2003; see below; Connolly et al., 2000; see below; Hachicha et al., 2007; see below; Joint Task Force on the Management of Valvular Heart Disease of the European Society of C, European Association for Cardio-Thoracic S et al., 2012; see below). Clinical outcome of the different groups has since been thoroughly examined. LG has been identified as independent predictor of reduced cardiac event free survival and mortality (Lancellotti et al., 2012; see below). Carabello et al. wondered, if it was an underlying cardiomyopathy that led to this difference in clinical outcome, representing “two distinct groups, rather than opposite ends of a spectrum”.

### Determination of Low-Gradient Patients by Echocardiography:

Echocardiography was performed using the Vivid 7 System (GE Vingmed Ultra-sound) with a 3.5-NHz transducer. LV end-diastolic and end-systolic dimensions, as well as end-diastolic thickness of the septum and posterior wall, were measured from M-mode parasternal LV long axis images. EF was calculated using the modified Simpson's biplane method. Continuous-wave Doppler recordings for assessing maximal aortic valve velocity were derived using the multiwindow interrogation. Peak aortic valve and LV outflow tract velocities and gradients were measured using continuous- and pulsed-wave Doppler. Aortic valve area was calculated by the continuity equation according to American Heart Association and European Society of Cardiology guidelines. Patients were grouped in high-gradient (≥40 mmHg) and low transvalvular pressure gradient (<40 mmHg) (Bonow et al., 2006; see below; Vahanian et al., 2007; see below). In patients with low-flow/low-gradient stenosis (reduced EF; gradient, <40 mmHg; aortic valve area, <1.0 cm²), the severity of aortic valve stenosis (AS) may be particularly difficult to assess because compromised aortic valve opening may be attributable to either significant valvular stenosis or to poor LV function. In such cases, a low-dose dobutamine stress echo was performed to confirm true severe stenosis. Only patients with a true stenosis were included. Biopsy samples of the basal septum were taken during aortic valve replacement. Informed consent was obtained from all patients. The study was reviewed and approved by the ethics committee of the Medical Faculty of the University of Wurzburg and was conducted according the principles outlined in the Declaration of Helsinki.

### REFERENCES

Nkomo V T, Gardin J M, Skelton T N, Gottdiener J S, Scott C G, Enriquez-Sarano M. Burden of valvular heart diseases: A population-based study. Lancet. 2006; 368:1005-1011
Carabello B A, Green L H, Grossman W, Cohn L H, Koster J K, Collins J J, Jr. Hemodynamic determinants of prognosis of aortic valve replacement in critical aortic stenosis and advanced congestive heart failure. Circulation. 1980; 62:42-48
Monin J L, Monchi M, Kirsch M E, Petit-Eisenmann H, Baleynaud S, Chauvel C, Metz D, Adams C, Quere J P, Gueret P, Tribouilloy C. Low-gradient aortic stenosis: Impact of prosthesis-patient mismatch on survival. European heart journal. 2007; 28:2620-2626
Monin J L, Quere J P, Monchi M, Petit H, Baleynaud S, Chauvel C, Pop C, Ohlmann P, Lelguen C, Dehant P, Tribouilloy C, Gueret P. Low-gradient aortic stenosis: Operative risk stratification and predictors for long-term outcome: A multicenter study using dobutamine stress hemodynamics. Circulation. 2003; 108:319-324
Connolly H M, Oh J K, Schaff H V, Roger V L, Osborn S L, Hodge D O, Tajik A J. Severe aortic stenosis with low transvalvular gradient and severe left ventricular dysfunction: Result of aortic valve replacement in 52 patients. Circulation. 2000; 101:1940-1946
Hachicha Z, Dumesnil J G, Bogaty P, Pibarot P. Paradoxical low-flow, low-gradient severe aortic stenosis despite preserved ejection fraction is associated with higher afterload and reduced survival. Circulation. 2007; 115:2856-2864
Joint Task Force on the Management of Valvular Heart Disease of the European Society of C, European Association for Cardio-Thoracic S, Vahanian A, Alfieri O, Andreotti F, Antunes M J, Baron-Esquivias G, Baumgartner H, Borger M A, Carrel T P, De Bonis M, Evangelista A, Falk V, Jung B, Lancellotti P, Pierard L, Price S, Schafers H J, Schuler G, Stepinska J, Swedberg K, Takkenberg J, Von Oppell U O, Windecker S, Zamorano J L, Zembala M. Guidelines on the management of valvular heart disease (version 2012). European heart journal. 2012; 33:2451-2496
Lancellotti P, Magne J, Donal E, Davin L, O'Connor K, Rosca M, Szymanski C, Cosyns B, Pierard L A. Clinical outcome in asymptomatic severe aortic stenosis: Insights from the new proposed aortic stenosis grading classification. Journal of the American College of Cardiology. 2012; 59:235-243
Bonow R O, et al.; American College of Cardiology/American Heart Association Task Force on Practice Guidelines; Society of Cardiovascular Anesthesiologists; Society for Cardiovascular Angiography and Interventions; Society of Thoracic Surgeons (2006) ACC/AHA 2006 guidelines for the management of patients with valvular heart disease: A report of the American College of Cardiology/American Heart Association Task Force on Practice Guidelines (writing committee to revise the 1998 Guidelines for the Management of Patients With Valvular Heart Disease): Developed in collaboration with the Society of Cardiovascular Anesthesiologists: Endorsed by the Society for Cardiovascular Angiography and Interventions and the Society of Thoracic Surgeons. Circulation 114(5):e84-e231.
Vahanian A, et al.; Task Force on the Management of Valvular Hearth Disease of the European Society of Cardiology; ESC Committee for Practice Guidelines (2007) Guidelines on the management of valvular heart disease: The Task Force on the Management of Valvular Heart Disease of the European Society of Cardiology. Eur Heart J 28(2):230-268.

### Data on RKIP and Aortic Stenosis Patients:

We analyzed the phosphorylation status of RKIP in heart failure, additional myocardial samples of aortic stenosis patents of different patient entities (severe aortic stenosis with low or high transvalvular gradients) with regard to RKIP expression and regulation and the correlated these data with the cardiac performance of these patients.

The analysis of murine (after 3 weeks of TAC-induced pressure overload) and human heart failure (cardiac explants of transplant patients) samples revealed that not only RKIP expression but also RKIP phosphorylation is upregulated in failing hearts.

To address the expression and regulation of RKIP in a broader context, we analyzed RKIP expression and phosphorylation in left ventricular myocardium of patients with aortic stenosis (AS; aortic valve area <1 cm²). Low gradient AS may be considered as a more systemic disease with vascular comorbidities like coronary heart disease, whilst in most patients with a high gradient AS calcified aortic valves are the major problem and other comorbidities are not common (Ruppert et al. Proc Natl Acad Sci USA 2013, 110: 7440-7445), and unpublished clinical oberservations by Weidemann et al.).

Strikingly, we found that RKIP expression and phosphorylation were only upregulated in high gradient AS patients, whereas low gradient AS patients seem to be an example for a patient entity that does not involve the upregulation of RKIP expression in heart failure and even shows a significant reduction of RKIP phosphorylation (FIG. 9). Thus, there are certain entities of heart failure patients that show downregulation in RKIP function.

As a further patient population, we have analyzed myocardium from patients with terminal heart failure due to ischemic cardiomyopathy (ICM). In these patients, heart failure was caused by coronary heart disease, usually triggered by a history of myocardial infarction. Patients with low gradient AS have in common with ICM patients a high prevalence of coronary heart disease. Interestingly, in ICM patients RKIP was neither upregulated nor activated (by phosphorylation) (FIG. 10). These results support the view that RKIP expression and phosphorylation may occur differently in different heart diseases and may modulate their severity. Furthermore, the results suggest that the underlying cause of a heart disease may determine RKIP regulation. Patient with defects in RKIP upregulation/activation (i.e. low-gradient aortic stenosis patients) are the patient entity that will profit most from an RKIP therapy or RKIP activating strategy or RKIP-like therapeutic strategy.

Recent clinical data in patients with severe AS indicate that high gradient AS is associated with better myocardial function than low gradient AS (Lancellotti et al., Journal of the American College of Cardiology 2012, 59:235-243; Hellmann et al., Journal of the American College of Cardiology 2011, 58:402-412; Cramariuc et al., Heart 2010, 96:106-112, Supplementary Table 12). Interestingly, a better contractility was associated with a significant induction of RKIP, while in low gradient AS with compromised contractile function RKIP phosphorylation was even downregulated. These data support the contention that low gradient patient would profit most from a “RKIP” Therapy that improves cardiac contractility but without long-term adverse effects.

SUPPLEMENTARY TABLE 12

| | Severe aortic | Severe aortic |
|---|---|---|
| | stenosis | stenosis |
| | low-gradient | high-gradient |
| Aortic valve area | 0.73 ± 0.047 | 0.73 ± 0.034 |
| (cm²) | | |
| Mean aortic gradient | 35 ± 1.6 | 62 ± 4.8* |
| (mmHg) | | |
| Ejection fraction | 52 ± 5.2 | 59 ± 2.1 |
| (%) | | |
| MAPSE | 7.4 ± 0.48 | 10 ± 0.44* |
| (mm) | | |
| n | 9 | 16 |
| Global strain | 7.48 ± 1.164 | 12.59 ± 0.600* |
| (%) | | |
| n | 5 | 15 |

*<0.05 versus low-gradient
MAPSE (=mitral annular plane systolic excursion) describes a regional deformation parameter and is a parameter for longitudinal cardiac performance that detects functional deficits under conditions where ejection fraction can still be normal (Lancellotti et al., Journal of the American College of Cardiology 2012, 59: 235-243; Herrmann et al., Journal of the American College of Cardiology 2011, 58: 402-412; Cramariuc et al., Heart 2010, 96: 106-112).

### RKIP-Concept:

RKIP improves cardiac function by improving cardiac contractility and relaxation (FIG. 1a-f) (via activation of b1-adrenergic receptors; FIG. 4a-c, e) and by protecting the heart at the same time from adverse effects (mediated via b2-adrenergic receptors (FIG. 4h-j), that are chronically activated in the Gi-coupled modus; Suppl FIG. 9j-m, Suppl FIG. 10e). The protection of adverse effects includes interstitial fibrosis, cell death, diastolic calcium leak and ectopic heart beats. The protection is mediated by b2-AR activation coupled to Gi proteins (the molecular effects are PTX (inhibits Gi proteins)-sensitive), which leads to a locally restricted protection from hyperphosphorylation/activation of proteins associated with arrhythmias and heart failure (i.e t-tubular proteins as the ryanodine receptor and the L-type calcium channel; FIG. 5, Suppl. FIGS. 9 and 10). The activation of the b-adrenergic receptors is preserved in the presence of RKIP in heart failure (Suppl FIG. 11) and AAV9-mediated RKIP-therapy rescues cardiac function even in the absence of endogenous RKIP in RKIP-KO mice.

RKIP is a receptor-selective GRK2 inhibitor (it does not affect cytosolic GRK2 targets).

### Accordingly, RKIP is Useful in the Following Therapies:

AAV-mediated gene therapy
induction of RKIP expression and activation (S153-phosphorylation) (see below)
identification of RKIP/GRK interaction site: N-terminus of GRK1 1-185AA (Lorenz et al. Nature 2003, 426:574-579) was identified in 2003. AA54-185 is now been found to built a solid interaction with RKIP so that the complex will may serve for crystallization and modeling of RKIP-like GRK2-inhibitory compounds.

### Possible RKIP-“Activators”

We tested an anti-CD20 antibody (such as rituximab) and an anti-VEGF antibody (such as bevacizumab) and several other therapeutic antibodies (obtained from the hospital pharmacy, Uniklinik Wiirzburg) on RKIP expression and RKIP activation (Ser153 phosphorylation) in neonatal rat cardiomyocytes as well as cardiomyocyte contractility.

We found an up-regulation of RKIP expression when treated with 75 μg/ml of an anti-EGFR antibody (in particular panitumumab). We identified an even more prominent up-regulation of RKIP when treated with an anti-CD20 antibody (in particular rituximab). The most up-regulation of RKIP expression was observed after treatment with an anti-VEGF antibody (in particular bevacizumab) (FIG. 11).

We then analyzed for “active” RKIP (Ser153-phosphorylation). This was increased with an anti-CD20 antibody (in particular rituximab) (FIG. 12 and FIG. 13) as well as with an anti-VEGF antibody (in particular bevacizumab) (FIG. 12 and FIG. 14).

To analyse whether these compounds also affect cardiomyocyte contractility, we compared the contractility of neonatal rat cardiomyocates (NRCM) in the presence of an anti-CD20 antibody (such as rituximab) and RKIP overexpression. As well as RKIP overexpression, the anti-CD20 antibody (in particular rituximab) increased NRCM contractility in the presence of isoprenaline.

### Methods

### Isolation of Rat Cardiomyocytes and Contractile Activity

Neonatal rat cardiomyocytes were isolated as described (Lorenz et al. Nature 2003, 426:574-579; Vidal et al., Cardiovasc. Res. 2012, 96:255-264). Cardiomyocytes were transduced by adenoviruses or treated with indicated antibodies. To monitor the contractile activity of cardiomyocytes, live cells were examined on a Zeiss Axiovert 135 microscope. Cells held thermostatically at 33° C. were observed in MEM, and the number of beats per minute was determined before and 2 min after stimulation with 50 nM isoprenaline by an observer who was blind to the experimental protocol studied.

The present invention refers the following nucleotide and amino acid sequences:

SEQ ID NO: 1:
Nucleotide sequence of the forward primer for verifying homozygous
deletion of RKIP; the primer sequence matches the sequence around
the insertion site of the trapping vector (5′→3′)
GCTTTCCAGGCCTCTCAGTGTTCATCAG
SEQ ID NO: 2:
Nucleotide sequence of the reverse primer for verifying homozygous
deletion of RKIP; the primer sequence matches the sequence around
the insertion site of the trapping vector (5′→3′)
CCCGCCCATCCTGCCCATAGG
SEQ ID NO: 3:
Nucleotide sequence of the GAPDH forward primer (5′→3′)
TGGCAAAGTGGAGATTGTTG
SEQ ID NO: 4:
Nucleotide sequence of the GAPDH reverse primer (5′→3′)
CATTATCGGCCTTGACTGTG
SEQ ID NO: 5:
Nucleotide sequence of the BNP forward primer (5′→3′)
GGATCGGATCCGTCAGTCGTT
SEQ ID NO: 6:
Nucleotide sequence of the BNP reverse primer (5′→3′)
AGACCCAGGCAGAGTCAGAAA
SEQ ID NO: 7:
Nucleotide sequence of the ANF forward primer (5′→3′)
ACAGATCTGATGGATTTCAAGAACCTG
SEQ ID NO: 8:
Nucleotide sequence of the ANF reverse primer (5′→3′)
AGTGCGGCCCCTGCTTCCTCA
SEQ ID NO: 9:
Nucleotide sequence of the COL3α1 forward primer (5′→3′)
AAACAGCAAATTCACTTACAC
SEQ ID NO: 10:
Nucleotide sequence of the COL3α1 reverse primer (5′→3′)
ACCCCCAATGTCATAGG
SEQ ID NO: 11:
Amino acid sequence of human RKIP
>sp|P30086 PEBP1_HUMAN Phosphatidylethanolamine-binding protein 1
OS = Homo sapiens GN = PEBP1 PE = 1SV = 3
MPVDLSKWSGPLSLQEVDEQPQHPLHVTYAGAAVDELGKVLTPTQVKNRPTSISWD
GLDSGKLYTLVLTDPDAPSRKDPKYREWHHFLVVNMKGNDISSGTVLSDYVGSGPP
KGTGLHRYVWLVYEQDRPLKCDEPILSNRSGDHRGKFKVASFRKKYELRAPVAGTC
YQAEWDDYVPKLYEQLSGK
SEQ ID NO: 12:
Nucleotide sequence of human RKIP
>ENA|CAA59404|CAA59404.1 Homo sapiens (human) phosphatidylethanolamine
binding protein
ATGCCGGTGGACCTCAGCAAGTGGTCCGGGCCCTTGAGCCTGCAAGAAGTGGAC
GAGCAGCCGCAGCACCCGCTGCATGTCACCTACGCCGGGGCGGCGGTGGACGAG
CTGGGCAAAGTGCTGACGCCCACCCAGGTTAAGAATAGACCCACCAGCATTTCGT
GGGATGGTCTTGATTCAGGGAAGCTCTACACCTTGGTCCTGACAGACCCGGATGC
TCCCAGCAGGAAGGATCCCAAATACAGAGAATGGCATCATTTCCTGGTGGTCAAC
ATGAAGGGCAATGACATCAGCAGTGGCACAGTCCTCTCCGATTATGTGGGCTCGG
GGCCTCCCAAGGGCACAGGCCTCCACCGCTATGTCTGGCTGGTTTACGAGCAGGA
CAGGCCGCTAAAGTGTGACGAGCCCATCCTCAGCAACCGATCTGGAGACCACCG
TGGCAAATTCAAGGTGGCGTCCTTCCGTAAAAAGTATGAGCTCAGGGCCCCGGTG
GCTGGCACGTGTTACCAGGCCGAGTGGGATGACTATGTGCCCAAACTG
TACGAGCAGCTGTCTGGGAAGTAG
SEQ ID NO: 13:
Amino acid sequence of rat RKIP
>sp|P31044|PEBP1_RAT Phosphatidylethanolamine-binding protein 1
OS = Rattus norvegicus GN = Pebp1 PE = 1SV = 3
MAADISQWAGPLSLQEVDEPPQHALRVDYGGVTVDELGKVLTPTQVMNRPSSISWD
GLDPGKLYTLVLTDPDAPSRKDPKFREWHHFLVVNMKGNDISSGTVLSEYVGSGPPK
DTGLHRYVWLVYEQEQPLNCDEPILSNKSGDNRGKFKVESFRKKYHLGAPVAGTCF
QAEWDDSVPKLHDQLAGK
SEQ ID NO: 14:
Nucleotide sequence of rat RKIP
>ENA|CAA50708|CAA50708.1 Rattus norvegicus (Norway rat)
phosphatidylethanolamine-binding protein
ATGGCCGCCGACATCAGCCAGTGGGCCGGGCCGCTGTCATTACAGGAGGTGGAT
GAGCCGCCCCAGCACGCCCTGAGGGTCGACTACGGCGGAGTAACGGTGGACGAG
CTGGGCAAAGTGCTGACGCCCACCCAGGTCATGAATAGACCAAGCAGCATTTCAT
GGGATGGCCTTGATCCTGGGAAGCTCTACACCCTGGTCCTCACAGACCCCGATGC
TCCCAGCAGGAAGGACCCCAAATTCAGGGAGTGGCACCACTTCCTGGTGGTCAA
CATGAAGGGCAACGACATTAGCAGTGGCACTGTCCTCTCCGAATACGTGGGCTCC
GGACCTCCCAAAGACACAGGTCTGCACCGCTACGTCTGGCTGGTGTATGAGCAGG
AGCAGCCTCTGAACTGTGACGAGCCCATCCTCAGCAACAAGTCTGGAGACAACC
GCGGCAAGTTCAAGGTGGAGTCCTTCCGCAAGAAGTACCACCTGGGAGCCCCGG
TGGCCGGCACGTGCTTCCAGGCAGAGTGGGATGACTCTGTGCCCAAGCTG
CACGATCAGCTGGCTGGGAAGTAG
SEQ ID NO: 15:
Amino acid sequence of mouse RKIP
>sp|P70296|PEBP1_MOUSE Phosphatidylethanol amine-binding
protein 1 OS = Mus musculus GN = Pebp1 PE = 1 SV = 3
MAADISQWAGPLCLQEVDEPPQRALRVDYAGVTVDELGKVLTPTQVMNRPSSISWD
GLDPGKLYTLVLTDPDAPSRKDPKFREWHHFLVVNMKGNDISSGTVLSDYVGSGPPS
GTGLHRYVWLVYEQEQPLSCDEPILSNKSGDNRGKFKVETFRKKYNLGAPVAGTCY
QAEWDDYVPKLYEQLSGK
SEQ ID NO: 16:
Nucleotide sequence of mouse RKIP
>ENA|BAB03276|BAB03276.1 Mus musculus (house mouse) hippocampal
cholinergic neuro stimulating peptide precursor protein
ATGGCCGCCGACATCAGCCAGTGGGCCGGGCCCTTGTGCTTGCAGGAGGTGGAC
GAGCCGCCCCAGCACGCCCTGCGGGTCGACTACGCCGGGGTGACGGTGGACGAG
CTGGGCAAAGTGCTAACGCCCACCCAGGTTATGAACAGGCCCAGCAGCATTTCAT
GGGACGGCCTTGATCCTGGGAAACTCTACACCCTGGTCCTCACAGACCCCGATGC
TCCCAGCAGGAAGGATCCCAAATTCAGGGAGTGGCACCACTTCCTGGTGGTCAAC
ATGAAGGGTAATGACATTAGCAGTGGCACTGTCCTCTCAGATTATGTGGGCTCCG
GGCCTCCCAGTGGCACAGGTCTCCACCGCTATGTCTGGCTGGTGTACGAGCAGGA
ACAGCCGCTGAGCTGCGACGAGCCCATTCTCAGCAACAAGTCTGGAGACAATCG
CGGCAAGTTCAAGGTGGAGACCTTCCGCAAGAAGTATAACCTGGGAGCCCCGGT
GGCGGGCACGTGCTACCAAGCCGAGTGGGATGACTATGTGCCCAAGCTG
TACGAGCAGCTGTCAGGGAAGTAG

1. (canceled)

2. A method of medical intervention/treatment of a patient in need thereof, said method comprising administering to said patient a medically active amount of a compound selected from the group consisting of:
a Raf kinase inhibitor protein (RKIP) or a functional fragment or a functional variant thereof, or a nucleic acid molecule coding for said RKIP or said functional fragment of functional variant); and
a RKIP agonist;
wherein said patient suffers from or is likely to suffer from a low transvalvular pressure gradient (Pₘₑₐₙ).

3. The method of claim 2, wherein said patient suffers from or is likely to suffer from aortic valve stenosis (AS), preferably low gradient AS (LG/AS), chemical cardiomyopathy and/or ischemia.

4. A method of
(i) treating, protecting from or preventing heart failure;
(ii) long-term increasing of cardiac contractile force;
(iii) maintaining cardiac function;
(iv) treating, protecting from or preventing pressure overload-induced cardiac failure;
(v) preventing heart failure;
(vi) inducing increased cardiac contractility;
(vii) inducing persistent positive inotropy;
(viii) treating or preventing irregular heartbeat/arrhythmia;
(ix) reducing pathological hypertrophy;
(x) inducing or increasing physiological hypertrophy; and/or
(xi) converting pathological hypertrophy into physiological hypertrophy in a patient, said method comprising administering to said patient a pharmaceutically effective amount of:
a Raf kinase inhibitor protein (RKIP) or a functional fragment or a functional variant thereof or a nucleic acid molecule coding for said RKIP or said functional fragment of functional variant; or
a RKIP agonist.

5. The method of claim 4, wherein said heart failure is and/or said patient suffers from or is likely to suffer from a low transvalvular pressure gradient.

6. The method of claim 4, wherein said patient suffers from or is likely to suffer from aortic valve stenosis (AS), preferably low gradient AS (LG/AS), chemical cardiomyopathy and/or ischemia.

7. The method of claim 2, wherein the low transvalvular pressure gradient (Pₘₑₐₙ) is ≤ or <60 mmHg, preferably ≤ or <50 mmHg, preferably ≤ or <40 mmHg (most preferred) or even ≤ or <30 mmHg.

8. The method of claim 2, wherein the aortic valve area is ≤ or <1.4 cm², preferably ≤ or <1.4 cm², preferably ≤ or <1.0 cm² (most preferred) or even ≤ or <0.8 cm².

9. The method of claim 2, wherein said heart failure is and/or said patient suffers from or is likely to suffer from low-flow/low-gradient stenosis.

10. The method of claim 2, wherein said RKIP, said fragment or said variant thereof is selected from the group consisting of
(a) a polypeptide which comprises or consists of the amino acid sequence as depicted in SEQ ID NO. 11, 13 or 15 or as available via the database entry NCBI Reference Sequence: NP_002558.1 (human); UniProt ID: P30086 (human); NCBI Reference Sequence: NP_058932.1 (rat) or UniProt ID: P31044-1 (rat);
(b) a polypeptide which comprises or consists of an amino acid sequence being at least 30%, preferably at least 40%, preferably at least 50%, preferably at least 60%, preferably at least 70%, preferably at least 80%, preferably at least 85%, preferably at least 90%, preferably at least 95%, preferably at least 97%, preferably at least 98%, preferably at least 99%, identical to a polypeptide of (a);
(c) a polypeptide which comprises or consists of an amino acid sequence encoded by a nucleic acid molecule hybridizing to the complementary strand of a nucleic acid molecule (e.g. as depicted in SEQ ID NO. 12, 14, 16) encoding the polypeptide of (a) or (b); and
(f) a polypeptide which comprises or consists of a fragment of the polypeptide of any one of (a) to (c).

11. The method of claim 2, wherein said RKIP agonist is selected from the group consisting of
(a) RKIP, said fragment or said variant thereof as defined in claim 2;
(b) a nucleic acid molecule encoding RKIP, said fragment or said variant thereof as defined in claim 2;
(c) a (n expression) vector comprising the nucleic acid molecule of (a or b); and
(d) an CD20 inhibitor/antagonist (e.g. Rituximab) or an VEGF inhibitor/antagonist (e.g. Bevacizimab); and
(e) an amino acid sequence that interacts with RKIP.

12.-15. (canceled)

16. The method of claim 2, which is to be administered chronically/in a long-term mode and/or at a low dose.

17. The method of claim 16, wherein the administration in a chronic/long-term mode is an administration every 1-4, preferably 2-3, weeks or even only every 2-3 months over a period of at least 1, 3 or 6 month, up to 1, 2, 3, 4, 5, 6, 7, 8, 9, 10 ore even more years.

18. The method of claim 16, wherein the administration at a low dose is a dose of about 1 to 50 mg/kg, 5 to 20 mg/kg, 8 to 17 mg/kg, 10 to 15 mg/kg bodyweight (bw) every 1-4, preferably 2-3, weeks or even only every 2-3 months.

19. The method of claim 2, wherein said compound is administered so that an increase of RKIP activity/expression and/or an activation of RKIP is achieved.

20. The method of claim 19, wherein said increase of RKIP activity/expression and/or an activation of RKIP results in a 1-15 fold RKIP activity/expression in the patient.

21. (canceled)

22. A method of medical intervention/treatment of a patient in need thereof, said method comprising administering to said patient a medically active amount of a compound selected from the group consisting of:
an antagonist of CD20 and/or an anti-CD20 molecule,
a VEGF antagonist; or
an inhibitor of VEGF related tyrosine kinases
wherein said patient suffers from or is likely to suffer from a low a heart failure.

23. The method of claim 22, wherein said patient suffers from or is likely to suffer from and/or said heart failure is aortic valve stenosis (AS), preferably low gradient AS (LG/AS), chemical cardiomyopathy and/or ischemia.

24. The method of claim 22, wherein said heart failure is and/or said patient suffers from or is likely to suffer from a low transvalvular pressure gradient.

25. The method of claim 22, wherein said heart failure is and/or said patient suffers from or is likely to suffer from
(i) irregular heartbeat/arrhythmia; and/or
(ii) pathological hypertrophy.

26. The method of claim 2, wherein said heart failure and/or said patient is characterized by a reduced RKIP expression and/or activity.
